(19) United States
(12) Patent Application Publication
(10) Pub. No.: US 2007/0135485 A1
(43) Pub. Date: Jun. 14, 2007

(54) Novel mch receptor antagonists

(76) Inventors: James Ronald Gillig, Indianpolis, IN (US); Michael Dean Kinnick, Indianapolis, IN (US); John Michael Morin, Brownsburg, IN (US); Antonio Navarro Martinez, Alcobendas (ES)

Correspondence Address: ELI LILLY & COMPANY, PATENT DIVISION, P.O. BOX 6288, INDIANAPOLIS, IN, 46206-6288, US

(21) Appl. No.: 10/575,815

(22) PCT Filed: Oct. 21, 2004
(86) PCT No.: PCT/US04/32314

§ 371 Date: Apr. 14, 2006

Related U.S. Application Data
(60) Provisional application No. 60/513,449, filed on Oct. 22, 2003.

(51) Int. Cl. A61K 31/454; A61K 31/4245; C07D 413/02

(52) U.S. Cl. 514/326; 514/364; 548/143; 546/208

(57) ABSTRACT

The present invention relates to a melanin concentrating hormone antagonist compound of formula I: or a pharmaceutically acceptable salt thereof, useful for the treamtment useful fog treating Type H diabetes and/or obesity.

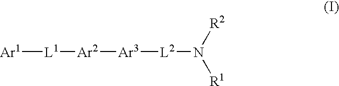

### FIELD OF INVENTION

The present invention is in the field of medicine, particularly in the treatment of obesity and diseases caused by, exacerbated by or related to obesity. More specifically, the present invention relates to antagonists of melanin concentrating hormone useful in the treatment of obesity and related diseases.

### BACKGROUND OF THE INVENTION

The affluence of the 90's along with the exponential increase in food production particularly in western and Asian economies has resulted in feeding patterns that lead to obesity. Obesity is defined as being excessively overweight. Excessive weight is generally characterized by excessive body fat, because unused energy is stored in the adipose tissues as fat.

However, obesity has an economic and social cost. Obese people an increasing proportion of most western societies are regarded as having out of control feeding habits; they may often have low self-esteem. Moreover, obese persons are more likely to have medical problems associated with or exacerbated by the excess body weight. Examples of medical conditions caused, exacerbated or triggered by excessive weight include bone fractures, pains in the knee joints, arthritis, increased risk of hypertension, artherosclerosis, stroke, etc.

It has been reported that the amount of feeding by MCH knock-out mice was significantly less than that of normal mice and that their body weights were less than those of normal mice, even though they behaved like normal mice in other respects (see Nature: vol, 396, 670 (1998). The current preferred treatment for obesity as well as Type II non-insulin dependent diabetes is diet and exercise with a view toward weight reduction and improved insulin sensitivity. Patient compliance, however, is usually poor. The problem is compounded by the fact that there are currently no approved medications that adequately treat either Type II diabetes or obesity.

PCT application number WO 01/21577 (JP00/06375) discloses compounds reportedly useful as antagonists of the MCH receptor. In particlular the WO 01/21577 application claims a compound of formula A 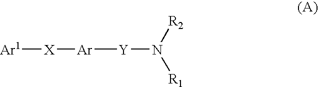
wherein:
Ar is a cyclic group that may have substituents;
X is a spacer having a main chain of 1 to 6 atoms;
Ar is a monocyclic aromatic ring which may be condensed with 4 to 8 membered aromatic ring, and may have further substituents;
R¹ and R² are independently hydrogen atom or a hydrocarbon group which may have substituents;
R¹ and R² together, together with the adjacent nitrogen atom and Y, may form a nitrogen containing hetero ring which may have substituents; or salts thereof.
Yet, there is a need for new and/or improved therapeutically effective agents useful as anatagonist of melanin releasing hormone to better control the dietary habits, minimize the preponderance of obesity and treat or ameliorate the effects of obesity including for example diabetes.

### SUMMARY OF INVENTION

The present invention relates to a compound of formula I: 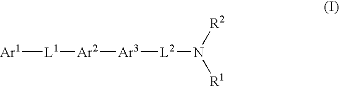
wherein:
Ar¹ is a cyclic group optionally substituted with one to five groups selected from C₁-C₈ alkyl, C₂-C₈ alkenyl, C₂-C₈ alkynyl, hydroxy, C_{1-C}₈ alkoxy, C₁-C₈ alkylaryl, phenyl, aryl, —O-aryl, heteroaryl, cycloalkyl, C₁-C₈ alkylcycloalkyl, cyano, —(CH₂)ₙNR⁶R⁶, C₁-C₈ haloalkyl, C₁-C₈ haloalkoxy, halo, (CH₂)ₙCOR⁶, (CH₂)ₙNR⁵SO₂R⁶, —(CH₂)ₙC(O)NR⁶R⁶, heterocyclic, and C₁-C₈ alkylheterocyclic; wherein the cycloalkyl, phenyl, aryl, and heterocyclic groups are each optionally substituted with one to three groups independently selected from hydroxy, C₁-C₈ alkoxyalkyl, C₁-C₈ haloalkoxy, C₁-C₈ alkyl, halo, C₁-C₈ haloalkyl, nitro, cyano, amino, carboxamido, phenyl, aryl, alkylheterocyclic, heterocyclic, and oxo;
L¹ is a bond or a divalent linker represented by the formula X₂—(CR³R⁴)ₘ—X₃ where X₂ is attached to Ar¹ and X₃ is attached to Ar² wherein R³ and R⁴ are independently selected from a bond, hydrogen, C₁-C₈ alkyl, C₂-C₈ alkylene, C₂-C₈ alkynyl, phenyl, aryl, C₁-C₈ alkylaryl; wherein the alkyl, alkenyl, phenyl, and aryl groups are optionally substituted with one to five substitutents independently selected from oxo, nitro, cyano, C₁-C₈ alkyl, aryl, halo, hydroxy, C₁-C₈ alkoxy, C₁-C₈ halaoalkyl, (CH₂)ₙC(O)R⁶, and (CH₂)ₙCONR⁶R⁶;
X₂ is independently oxygen, —CH, —CONH(CR³R⁴)ₘ, —NHCO(CR³R⁴)ₘ, —(CR³R⁴)ₘ, —CHR⁶, —NR⁵, S, SO, SO₂, —O(CR³R⁴)ₘ, or —S(CR³R⁴)ₘ;
X₃ is independently oxygen, —C, —CH, —CHR⁶, —(CR³R⁴)ₘ, —NR⁵, S, SO, or SO₂;
Ar² is a 5-member monocyclic heterocyclic aromatic group or positional isomer thereof, having 1, 2, or 3 heteroatoms independently selected from nitrogen, oxygen and sulfur; and wherein Ar² is optionally substituted with one to three substitutents independently selected from C₁-C₈ alkyl, C₂-C₈ alkenyl, C₂-C₈ alkynyl, hydroxy, C₁-C₈ alkoxy, C₁ -C₈ alkylaryl, phenyl, aryl, C₃-C₈ cycloalkyl, C₁-C₈ alkylcycloalkyl, cyano, C₁-C₈ haloalkyl, halo, (CH₂)ₙC(O)R⁶, (CH₂)ₙC(O)OR⁶, (CH₂)ₙNR⁵SO₂R⁶, (CH₂)ₙC(O)NR⁶R⁶, and C₁-C₈ alkylheterocyclic;
Ar³ is an optionally substituted bicyclic aromatic or non-aromatic group;
L² is a divalent linker represented by the formula X₄—(CR³R⁴)ₘ—X₅;
wherein X₄ is selected from the group consisting of C, —CH, CHR⁶, —CO, O, —NR⁵, —NC(O)—, —NC(S), —C(O)NR⁵—, —NR^{6′}C(O)NR⁶, —NR^{6′}C(S)NR⁶, —SO₂NR⁷, —NRSO₂R⁷, and —NR^{6′} C(NR⁵)NR⁶;
X₅ is selected from the group consisting of O, —CH₂, —CH, —O(CR³R⁴)m, NR³(CR³R⁴)ₘ, SO, SO₂, S, and SCH₂; wherein the group X₄—(CR³R⁴)ₘ—X₅ imparts stability to the compound of formula (1) and may be a saturated or unsaturated chain or divalent linker;
R¹ and R² are independently hydrogen, C₁-C₈ alkyl, C₂-C₈ alkenyl, C₃-C₈ cycloalkyl, C₁-C₈ alkylaryl, —C(O)C₁-C₈ alkyl, —C(O)OC₁-C₈ alkyl, C₁-C₈ alkylcycloalkyl,
(CH₂)ₙC(O)OR⁵, (CH₂)ₙC(O)R⁵, (CH₂)ₙC(O)NR⁶R⁶, and (CH₂)ₙNSO₂R⁵; wherein each of the alkyl, alkenyl, aryl are each optionally substituted with one to five groups independently selected from C₁-C₈ alkyl, C₂-C₈ alkenyl, phenyl, and alkylaryl; and wherein R¹ and R² may combine together, and with the nitrogen atom to which they are attached or with 0, 1, 2 or 3 atoms adjacent to the nitrogen atom to form a nitrogen containing heterocycle which may have 1, or 2 substituents independently selected from C₁-C₈ alkyl, C₂-C₈ alkenyl, C₃-C₈ cycloalkyl, C₁-C₈ alkylaryl, —C(O)C₁-C₈ alkyl, —C(O)OC₁-C₈ alkyl, C₁-C₈ alkylcycloalkyl,oxo, halo amino, and (CH₂)ₙC(O)NR⁶R^{6′};
R⁵ is hydrogen, CN, C₁-C₈ alkyl, C₂-C₈ alkenyl, C₅-C₈ alkylaryl, (CH₂)ₙNSO₂C₁-C₈ alkyl, (CH₂)ₙNSO₂phenyl, (CH₂)ₙNSO₂aryl, —C(O)C₁-C₈ alkyl, or —C(O)OC₁-C₈ alkyl; and
R⁶ and R^{6′} are each independently hydrogen, C₁-C₈ alkyl, phenyl, aryl, C₁-C₈alkylaryl, C₁-C₈alkylcycloalkyl, or C₃-C₈cycloalkyl;
R⁷ is hydrogen, C₁-C₈ alkyl, phenyl, aryl, C₁-C₈alkylaryl, or C₃-C₈cycloalkyl, and wherein m is an integer from 1 to 8; and n is an integer from 0 to 8;
   or a pharmaceutically acceptable salt, solvate, racemate, or enantiomer diastereomer or mixture of diastereomers thereof.

The present invention also relates to a method for treating obesity.

The present invention also relates to a method for antagonizing the release of melanin concentrating hormone employing a compound of formula I.

The present invention is also related to the use of a compound of formula I for the manufacture of a medicament for treating obesity as well as for treating or ameliorating the effects of diseases related to, adjunct to, or caused by obesity such as for example, high blood pressure, stroke, diabetes, hyperlipdemia, hyperglycemia, or hyperlipoproteinenamia.

The present invention is also related to the treatment and or prevention of obesity and related diseases including diabetes mellitus, hyperglycemia, hyperlipidemia, hypertriglyceridemia, hypercholesterolemia, atherosclerosis of coronary, aneurisms of cerebrovascular and peripheral arteries, gastrointestinal disorders including peptic ulcer, esophagitis, gastritis and duodenitis, (including that induced by H. pylori), intestinal ulcerations (including inflammatory bowel disease, ulcerative colitis, Crohn's disease and proctitis) and gastrointestinal ulcerations, neurogenic inflammation of airways, including cough, asthma, depression, prostate diseases such as benign prostate hyperplasia, irritable bowel syndrome and other disorders needing decreased gut motility, diabetic retinopathy, neuropathic bladder dysfunction, elevated intraocular pressure and glaucoma and non-specific diarrhea dumping syndrome.

In another embodiment, the pharmaceutical formulations of the present invention may be adapted for use in treating obesity, diabetes and related diseases.

### DETAILED DESCRIPTION

For the purposes of the present invention, as disclosed and/or claimed herein, the following terms are defined below.

In general valency is conserved for all compounds disclosed and/or claimed herein unless otherwise specified. Thus, the requirement and availability of hydrogen atoms to satisfy valency requirements is feature of this document that is known to or obvious to one of skill in the art.

The term “halo” represents fluoro, chloro, bromo; or iodo.

The term “C₁-C₈ alkyl” or “C₁-C₈ alkyl” represents a straight or branched hydrocarbon moiety having from one to eight carbon atoms, e.g., methyl, ethyl, n-propyl, isopropyl, n-butyl, isobutyl, sec-butyl, t-butyl, pentyl, n-hexyl, n-octyl, and the like. The term “C₁-C₄ alkyl” refers specifically to methyl, ethyl, n-propyl, isopropyl, n-butyl, isobutyl, sec-butyl, and t-butyl. A “C₁-C₄ haloalkyl” group is a C₁-C₄ alkyl moiety substituted with up to six halo atoms, preferably one to three halo atoms. An example of a haloalkyl group is trifluoromethyl.

A “C₁-C₆ alkoxy” group is a C₁-C₆ alkyl moiety connected through an oxy linkage.

The term cycloalkyl has its common meaning and is limited by the nymber of carbon atoms defining its size, i.e. C₃-C₈ cycloalkyl refers to a 3 to 8 member (inclusive) cyclic alkyl group including cylcobutane, cyclopentane, cyclohexane, cycloheptane and cyclooctane.

The term “cycloalkenyl” has its common meaning and is limited by the number of carbon atoms defining its size i.e. C₃-C₈ cycloalkenyl. Specific examples of C₃-C₈ cycloalkenyl include cyclopropenyl, cyclobutenyl, cyclopentenyl, cyclohexenyl, cycloheptenyl, and cyclooctenyl.

The term “cyclic” as used herein refers to substituted or unsubstituted aromatic and non-aromatic hydrocarbon groups, and substituted or unsubstituted aromatic and non-aromatic heterocyclic groups. Cyclic groups may also be monocyclic, bicyclcic or polycyclic. Examples of aromatic groups include for example benzene, thiophene, furan, pyrrole, imidazole, pyrazole, thiazole, isothiazole, oxazole, isoxazole, pyridine, pyrimidine, pyrazine, pyrimidine, pyridazine, 1,2,4-oxadiazole, 1,3,4-oxadiazole, 1,2,4-thiadiazole, and 1,3,4-thiadiazole, Other examples of cyclic groups include cyclopropenyl, cyclobutenyl, cyclopentenyl, cyclohexenyl, cycloheptenyl, cyclooctenyl, tetrahydrothiophene, tetrahydrofuran, pyrrolidine, imidazoline, imidazolidine, pyrazoline, pyrazolidine, tetrahydrothiazole, tetrahydroisothiazole, tetrohydrooxazole, tetrahydroisoxazole, piperidine, tetrahydropyridine, dihydropyridine, piperazine, morpholine, thiomorpholine, tetrahydropyrimidine, tetrahydropyridazine, hexamethyleneimine, benzofuran, benzimidazole, benzoxazole, benzothiazole, benzisothiazole, naphto[2,3-b]thiphene, isoquinoline, quinoline, indole, quinoxaline, phenathridine, phenothiadine, phenoxazine, naphthylidene, quinazoline, carbazole, b-carboline, acridine, phenazine, phthalimide, and thioxanthene each of which may be optionally substituted.

Examples of bicyclic aromatic or non-aromatic groups for Ar³ include C₉-C₁₄ bicyclic hydrocarbon aromatic or non-aromatic groups which may be optionally susbsituted. Examples of bicyclic heteroaromatic rings for Ar³ include for example, benzofuran, benzimidazole, benzoxazole, benzothiazole, benzoisothiazole, naphto[2,3-b]thiophene, isoquinoline, quinoline, indole, quinoxaline, naphthyl, tetrahydronapthyl, phenanthridine, phenothiadine, phenoxazine, naphthylidene, quinazoline, carbazole, b-carboline, acridine, phenazine, phthalimido, and thioxanthene all of which may be optionally substituted. Optional substituents on the bicyclic group Ar³ include oxo, amino, hydroxy, oxo, amino, C₁-C₈ alkyl, C₂-C₈ alkenyl, C₂-C₈ alkynyl, phenyl, C₁-C₈ alkylaryl, C(O)C₁-C₈ alkyl, CO(O)C₁-C₈ alkyl, halo, and C₁-C₈ haloalkyl.

The term alkylcycloalkyl” as used herein refers to an alkylgroup on which a cycloalkyl group is substituted. Exemplary of alkylcycloalkyl groups are methylcyclopropyl, methylcyclohexyl, methylcycloheptyl, ethylcyclopropyl etc. The alkylcycloalkyl group may be optionally substituted.

The term “optionally substituted” as used herein means an optional substitution of one to three, preferably one or two groups independently selected from halo, hydroxy, oxo, cyano, nitro, phenyl, benzyl, triazolyl, tetrazolyl, 4,5-dihydrothiazolyl, halo, C₁-C₆ alkyl, C₁-C₄ haloalkyl, C₁-C₆ alkoxy, COR⁸, CONR⁸R⁹, CO₂R⁸, NR⁸R⁹, NR⁸COR⁹, NR⁸SO₂R⁹, OCOR⁹, OCO₂R⁸, OCONR⁸R⁹, SR⁸, SOR⁹, SO₂R⁹ and SO₂(NR⁸R⁹), where R⁸ is independently at each occurrence H, C₁-C₆ alkyl, phenyl or benzyl and R⁹ is independently at each occurrence C₁-C₆ alkyl, phenyl or benzyl.

The term “chain length” as used herein refers to the longest distance in number of atoms counting linearly from the first to the last atom constituting the group and traversing the main or longer length of the molecule. For example —OCH₂CH₂OCH₂— has a chain length of 5, while —OCH₂(4(3-fluorophenyl)CH₂CH₂— represented below 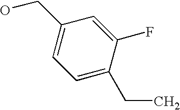
has a chain length of 8 counting from the oxygen to the terminal methylene group.

The term “heterocycle” or “heterocyclyl” or “heterocyclic” represent a stable, saturated, partially unsaturated, fully unsaturated or aromatic 4, 5 or 6 -member ring, said ring having from one to four heteroatoms that are independently selected from the group consisting of sulfur, oxygen, and nitrogen. The heterocycle may be attached at any point that affords a stable structure. Representative heterocycles include 1,3-dioxolane, 4,5-dihydro-1H-imidazole, 4,5-dihydrooxazole, furan, imidazole, imidazolidine, isothiazole, isoxazole, morpholine, oxadiazole, oxazole, oxazolidinedione, oxazolidone, piperazine, piperidine, pyrazine, pyrazole, pyrazoline, pyridazine, pyridine, pyrimidine, pyrrole, pyrrolidine, tetrazole, thiadiazole, thiazole, thiophene and triazole. The heterocycle is further optionally substituted with one to three, preferably one or two groups independently selected from halo, hydroxy, oxo, cyano, nitro, phenyl, benzyl, triazolyl, tetrazolyl, 4,5-dihydrothiazolyl, C₁-C₆ alkyl, C₁-C₄ haloalkyl, C₁-C₆ alkoxy, COR⁸, CONR⁸R⁹, CO₂R⁸, NR⁸R⁹, NR⁸COR⁹, NR⁸SO₂R⁹, OCOR⁹, OCO₂R⁸, OCONR⁸R⁹, SR⁸, SOR⁹, SO₂R⁹ and SO₂(NR⁸R⁹), where R⁸ is independently at each occurrence H, C₁-C₆ alkyl, phenyl or benzyl and R⁹ is independently at each occurrence C₁-C₆ alkyl, phenyl or benzyl.

The term alkylheterocyclic” as used herein refers to an alkyl group further substituted with a heterocyclic group. Examples of alkylheterocycles include but are not limited to 2-methylimidazoline, 2-methylindole, and 2-ethylthiophene.

The term “C₁-C₄ haloalkyl” refers to a C₁-C₄ alkyl group substituted with one, two, or three halogen atoms as possible and appropriate. Examples of C₁-C₄ haloalkyl include but are not limited to trifluoromethyl, chloroethyl, and 2-chloropropyl.

The term “suitable solvent” refers to any solvent, or mixture of solvents, inert to the ongoing reaction that sufficiently solubilizes the reactants to afford a medium within which to effect the desired reaction.

The term “patient” includes human and non-human animals such as companion animals (dogs and cats and the like) and livestock animals. Livestock animals are animals raised for food production. Ruminants or “cud-chewing” animals such as cows, bulls, heifers, steers, sheep, buffalo, bison, goats and antelopes are examples of livestock. The preferred patient of treatment is a human.

The terms “treating” and “treat”, as used herein, include their generally accepted meanings, i.e., preventing, prohibiting, restraining, alleviating, ameliorating, slowing, stopping, or reversing the progression or severity of a pathological condition, or sequela thereof, described herein.

The terms “preventing”, “prevention of”, “prophylaxis”, “prophylactic” and “prevent” are used herein interchangeably and refer to reducing the likelihood that the recipient of a compound of formula I will incur or develop any of the pathological conditions, or sequela thereof, described herein.

As used herein, the term “effective amount” means an amount of a compound of formula I that is capable of treating conditions, or detrimental effects thereof, described herein or that is capable of agonizing the β₃ receptor.

The term “pharmaceutically acceptable” is used herein as an adjective and means substantially non-deleterious to the recipient patient.

The term “formulation”, as in pharmaceutical formulation, is intended to encompass a product comprising the active ingredient(s) (compound(s) of formula I), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical formulations of the present invention encompass any composition made by admixing a compound of the present invention and a pharmaceutical carrier, or a compound of the formula I and a pharmaceutoically acceptable co-agonist useful for the treatment and/or prevention of obesity, diabetes and related diseases.

The term “unit dosage form” refers to physically discrete units suitable as unitary dosages for human subjects and other non-human animals (as described above), each unit containing a predetermined quantity of active material calculated to produce the desired therapeutic effect, in association with a suitable pharmaceutical carrier.

Because certain compounds of the invention contain an acidic moiety (e.g., carboxy), the compound of formula I may exist as a pharmaceutical base addition salt thereof. Such salts include those derived from inorganic bases such as ammonium and alkali and alkaline earth metal hydroxides, carbonates, bicarbonates, and the like, as well as salts derived from basic organic amines such as aliphatic and aromatic amines, aliphatic diamines, hydroxy alkamines, and the like.

Because certain compounds of the invention contain a basic moiety (e.g., amino), the compound of formula I can also exist as a pharmaceutical acid addition salt. Such salts include the salicylate, sulfate, pyrosulfate, bisulfate, sulfite, bisulfite, phosphate, mono-hydrogenphosphate, dihydrogenphosphate, metaphosphate, pyrophosphate, chloride, bromide, iodide, acetate, propionate, decanoate, caprylate, acrylate, formate, isobutyrate, heptanoate, propiolate, oxalate, malonate, succinate, suberate, sebacate, fumarate, maleate, 2-butyne-1,4 dioate, 3-hexyne-2, 5-dioate, benzoate, chlorobenzoate, hydroxybenzoate, methoxybenzoate, phthalate, xylenesulfonate, phenylacetate, phenylpropionate, phenylbutyrate, citrate, lactate, hippurate, β-hydroxybutyrate, glycolate, maleate, tartrate, methanesulfonate, propanesulfonate, naphthalene-1-sulfonate, naphthalene-2-sulfonate, mandelate and like salts. Preferred acid addition salts include the hydrochloride, nydrobromide, hydrogen sulfate, and oxalate salts.

### Preferred Compounds of the Invention

Certain compounds of the invention are particularly interesting and preferred. The following listing sets out several groups of preferred compounds. It will be understood that each of the listings may be combined with other listings to create additional groups of preferred compounds.

Preferred Ar¹

Preferred Ar¹ groups are cyclic groups selected from cycloalkyl and cycloalkene groups such as the group consisting of cyclopropyl, cyclobutyl, cyclopentyl, cyclohexyl, cycloheptyl, cyclooctyl, cyclopropenyl, cyclobutenyl, cyclopentenyl, cyclohexenyl, cycloheptenyl, and cyclooctenyl. Also preferred are,groups selected from tetrahydrothiophene, tetrahydrofuran, pyrrolidine, imidazoline, imidazolidine, pyrazoline, pyrazolidine, tetrahydrothiazole, tetrahydroisothiazole, tetrahydrooxazole, phenyl, tetrahydroisoxazole, piperidine, tetrahydropyridine, benzothiophene, benzofuran, naphthyl, dihydropyridine, piperazine, morpholine, thiomorpholine, tetrahydropyrimidine, tetrahydropyridazine, hexamethyleneirnine, each optionally substituted with C₁-C₆ alkyl, C₁-C₆ cycloalkyl, C₁-C₆ haloalkyl,hydroxy, alkoxyalkyl, cyano, halo, aryl, carboxamide, and C₁-C₆ carboxyalkyl. More preferred Ar¹ groups include cycloalkyl, cycloalkenyl, substituted or unsubstituted phenyl, benzothiophene, benzofuran and naphthyl. Particularly preferred Ar¹ groups include phenyl, benzothiophene, benzofuran, and naphthyl.

Preferred L¹ Groups

Preferred as L¹ are groups having between 3 to 8 carbon atoms in the main chain. Also preferred are L¹ groups selected from the group consisting of —CH₂—, —CH₂CH₂—, —CH₂CH₂CH₂—, —SCH₂—, —OCH₂—, —CH₂SCH₂—, —CH₂OCH₂—, —OCH₂CH₂SCH₂—, —OCH₂CH₂OCH₂—, —O(CH₂)₃SCH₂—, —OCH(Et)CH₂CH₂SCH₂, —OCH(iPr)CH₂CH₂SCH₂, —OCH(CH₃)CH₂CH₂SCH₂, —O(CH₂)₃SCH(CH₃)—, —O(CH₂)₂SCH(CF₃)—, —OCH₂CH(NO₂)SCH₂—, —OCH(CN)CH₂SCH₂, —OCH₂CH(NH₂)SCH₂—, —CH₂O(CH₂)₃CH₂O—, and —CH₂O(CH₂)₂CH₃O—

Preferred X₂ Group

Also preferred is an L¹ group having the formula X₂—(CR³R⁴)ₘ—X₃ wherein a preferred X₂ group is selected from O, S, and —NR⁶, and wherein R⁶ is selected from the group consisting of hydrogen, C₁-C₆ alkyl, C₂-C₆ alkenyl, C₃-C₈ cycloalkyl, phenyl, benzyl, C₁-C₈ alkylamine, and aryl.

Preferred X₃ Groups

Also preferred is an L¹ group wherein, for L¹ is X₂—(CR³R⁴)ₘ—X₃; X₃ is a group selected from —OCH₂, —SCH₂, —NR⁶C(O)CH₂, —NHCH₂, wherein R⁶ is selected from the group consisting of hydrogen, C₁-C₆ alkyl, C₂-C₆ alkenyl, C₃-C₈ cycloalkyl, phenyl, benzyl, and aryl. More preferred is an X₃ group selected from —OCH₂, and —SCH₂.

Also preferred is a compound of formula I wherein L¹ is X₂—(CR³R⁴)ₘ—X₃, and wherein the chain between X₂ and X₃ i.e., —(CR³R⁴)ₘ— is an alkyl chain of 3 to 8 carbon atoms, or an alkenyl chain of 3 to 8 carbon atoms and optionally contains an alkyl, phenyl, amino, or cycloalkyl group as a side chain.

Preferred Ar² Groups

A preferred Ar² group is a 5-member monocyclic aromatic heterocyclic group having 1, 2, or 3 heteroatoms selected from oxygen, sulfur, and nitrogen. More preferred is a heterocyclic group selected from furan, thiophene, pyrrole, oxazole, thiazole, imidazole, imidazoline, imidazolidine, pyrazole, 2-pyraziline, pyrazolidine, isoxazole, isothiazole, 1,3,4-oxadiazole, 1,2,3-triazole, 1,3,4-thiadiazole and 1,3,4-oxadiazole. Most preferred Ar² are the oxadiazolyl or oxazolyl groups, and positional isomers thereof.

Preferred Ar³ Groups

Most preferred Ar³ groups are bicyclic groups selected from the group consisting of naphthalene, indolyl, isoindolyl, indolinyl, benzo[B]furanyl, oxoindole, isoquinolone, tetrahydronapthyl, benzo[b]thiophenyl, 1H-indazolyl, benzimidazolyl, benzothiazolyl, quinolinyl, and isoquinolinyl wherein each may be optionally substituted with 1 to 3 substituents selected from C₁-C₆ alkyl, —SO₂R, SO2NHR, and benzyl. Most preferred Ar³ is a group selected from naphthyl, indolyl, benzofuran and positional isomers thereof, wherein each is optionally substituted preferably with C₁-C₆ alkyl, SO₂R, NH₂SO₂R, and CH₂SO₂NHR.

Preferred L² Groups

Preferred is an L² group selected from the group consisting of —OCH₂CH₂—, —O(CH₂)₃—, —CH₂, —CH₂CH₂, —CH₂CH₂CH₂, —CH═CH, —CH₂CH₂CH═CH— and X₄—(CR³R⁴)ₘ—X₅. The most preferred L² groups are —CH₂ and —CH₂CH₂.

Preferred X₄ Groups

Preferred X₄ groups include divalent groups, radicals, or fragments of the formula —C(O)NR⁶ wherein R⁶ is selected from the group consisting of hydrogen, C₁-C₆ alkyl, C₂-C₆ alkenyl, C₃-C₈ cycloalkyl, phenyl, benzyl, C₁-C₈ alkylamine, and aryl.

Also preferred is an X₄ group selected from O, S, —NR⁶C(O)NR⁶, —C(S)NR⁶, NR⁶C(S)NR⁶, NR⁶C(NR⁶)NR⁶, —NR⁶SO₂—, wherein R⁶ is independently selected from the group consisting of hydrogen, C₁-C₆ alkyl, C₂-C₆ alkenyl, C₃-C₈ cycloalkyl, phenyl, benzyl, C₁-C₈ alkylamine, and aryl.

Preferred X₅ Groups

Preferred is an X₅ group selected from —OCH₂, —SCH₂, O, —NR⁶C(O), —NR⁶C(S), —C(O)NR⁶, —C(S)NR⁶, NR⁶C(S)NR⁶, NC(NR⁶)N, NR⁶C(O)NR⁶, —NR⁶SO₂ wherein R⁶ is independently selected from the group consisting of hydrogen, C₁-C₆ alkyl, C₂-C₆ alkenyl, C₃-C₈ cycloalkyl, phenyl, benzyl, C₁-C₈ alkylamine, and aryl. More preferred is an X₅ group selected from —OCH₂, —SCH₂ and O.

Also preferred is a compound of formula I wherein the chain between X₄ and X₅ is preferably an alkyl chain of 2 to 8 carbon atoms, or an alkenyl chain of 2 to 8 carbon atoms and optionally containing an alkyl, phenyl, or cycloalkyl group as a side chain.

Preferred R¹ and R² Groups

Preferred R¹ and R² groups are independently selected from the group consisting of C₁-C₆ alkyl, C₂-C₆ alkenyl, C₃-C₈ cycloalkyl, C₃-C₈ alkylcycloalkyl, phenyl, benzyl, COR⁹, SO₂R⁹

Also preferred is a compound of the invention having R¹ and R² groups wherein the R¹ and R² groups combine with the nitrogen atom to which they are attached and with a carbon atom one or two atoms removed from the nitrogen atom to form a cycle such as for example, azepine, diazepine, pyridine, piperidine, indolyl, N-methylpyrrolidinyl, pyrrolidinyl, morpholino, piperidinyl, and the like.

Most preferred are R¹ and R² which singly or in combination with each other and/or the nitrogen atom to which they are attached form the groups independently selected from methyl, ethyl, propyl, isopropyl, isobutyl, cyclopentyl, cyclohexyl, N-morpholino, azepane, diazepine, pyridine, pyrrolidine, piperidine, N-methylpiperidine, and N-methylpiperazine.

Preferred compounds of the invention are compounds selected from the group consisting of:
Dimethyl-{6-[5-(2-phenoxy-ethylsulfanylmethyl)-[1,3,4]oxadiazol-2-yl]-benzofuran-2-ylmethyl}-amine oxalate,
Dimethyl-{5-[5-(2-phenoxy-ethylsulfanylmethyl)-[1,3,4]oxadiazol-2-yl]-benzofuran-2-ylmethyl}-amine oxalate, {1 -Methanesulfonyl-5-[5-(2-phenoxy-ethylsulfanylmethyl)-[1,3,4]oxadiazol-2-yl]-1H-indol-2-ylmethyl}-dimethyl-amine,
Dimethyl-{5-[5-(2-phenoxy-ethylsulfanylmethyl)-[1,3,4]oxadiazol-2-yl]-1H-indol-2-ylmethyl}-amine oxalate,
{1-Methanesulfonyl-6-[5-(2-phenoxy-ethylsulfanylmethyl)-[1,3,4]oxadiazol-2-yl]-1H-indol-2-ylmethyl}-dimethyl-amine,
Dimethyl-{6-[5-(2-phenoxy-ethylsulfanylmethyl)-[1,3,4]oxadiazol-2-yl]-1H-indol-2-ylmethyl}-amine,
Dimethyl-{1-methyl-6-[5-(2-phenoxy-ethylsulfanylmethyl)-[1,3,4]oxadiazol-2-yl]-1H-indol-2-ylmethyl}-amine oxalate,
Dimethyl-{5-[5-(2-phenoxy-ethylsulfanylmethyl)-[1,3,4]oxadiazol-2-yl]-1H-indol-3-ylmethyl}-amine oxalate,
Dimethyl-{6-[5-(2-phenoxy-ethylsulfanylmethyl)-[1,3,4]oxadiazol-2-yl]-1H-indol-3-ylmethyl}-amine maleate,
Dimethyl-{1-methyl-5-[5-(2-phenoxy-ethylsulfanylmethyl)-[1,3,4]oxadiazol-2-yl]-1H-indol-3-ylmethyl}-amine oxalate,
Dimethyl-{4-[5-(2-phenoxy-ethylsulfanylmethyl)-[1,3,4]oxadiazol-2-yl]-naphthalen-1-yl}-amine,
Dimethyl-{6-[5-(2-phenoxy-ethylsulfanylmethyl)-[1,3,4]oxadiazol-2-yl]-naphthalen-2-ylmethyl}-amine,
2-(2-Phenoxy-ethylsulfanylmethyl)-5-(6-pyrrolidin-1-ylmethyl-naphthalen-2-yl)-[1,3,4]oxadiazole maleate,
1-{6-[5-(2-phenoxy-ethylsulfanylmethyl)-[1,3,4]oxadiazol-2-yl]-naphthalen-2-ylmethyl}-piperidine,
2-(2-piperidinoethyl)-5-{2-[((2-phenoxyethyl)thio)methyl]-1,3,4-oxadiazol-5-yl}isoindolin-1-one,
2-{[(2-Phenoxyethyl)thio]methyl}-5-{3-hydroxymethyl-4-[((2-piperidinoethyl)amino)carbonyl]phenyl}-1,3,4-oxadiazolo,
2-(2-piperidinoethyl)-5-{2-[((2-phenoxyethyl)thio)methyl]-1,3,4-oxadiazol-5-yl}isoindolin-1-one, and pharmaceutically acceptable salt, solvate, enatiomer, prodrug, diastereomer or a mixture thereof.

### Preparing Compounds of the Invention

Compounds of formula I may be prepared as described in the following Schemes and Examples. Precursors to the compounds of the invention are prepared by methods known to one of skill in the art. The compounds employed as initial starting materials in the synthesis of the compounds of the invention are well known and, to the extent not commercially available, are readily synthesized by standard procedures commonly employed by one of ordinary skill in the art. More particularly, the compounds of the invention are produced in accordance with the General Methods 1 through 5 that are described in detail below, or analogous methods thereto. These reactions are often carried out in accordance with per se known methods, or analogous methods thereto. Examples of such known methods include the methods described in general reference texts such as Organic Functional Group Preparations, 2^{nd} Edition, 1989; Comprehensive Organic Transformations, VCH Publishers Inc, 1989; Compendium of Organic Synthetic Methods, Volumes 1-10, 1974-2002, Wiley Interscience; March's Advanced Organic Chemistry, Reactions Mechanisms, and Structure, 5^{th} Edition, Michael B. Smith and Jerry March, Wiley lnterscience, 2001, Advanced Organic Chemistry, 4^{th} Edition, Part B, Reactions and Synthesis, Francis A. Carey and Richard J. Sundberg, Kluwer Academic/Plenum Publishers, 2000, etc., and references cited therein.

### General Method 1: Coupling of the Basic Group

The compounds of Formula 3 can be prepared by the General Method 1, described in General Scheme 1, via coupling of a compound of Formula 2 containing a basic group with a group of Formula 1, where during the course of the coupling reaction the coupling groups are retained or lost to form the linker L² between the basic group and the phenyl ring. Ar¹, L¹, Ar², L², and basic group are defined as above. In the schemes that follow Ar³ of formula I has been depicted as a naphthyl group for convenience only and is not intended to be limiting. Nor are any depicted positional isomers intended to be limiting, as well. Also, Lₐ is defined as a group that when the coupling process occurs results in the formation of the linker L² defined above. Furthermore, in the schemes that follow, the group L¹ is depicted by the combination of group or groups interspacing or linking the groups Ar¹ and Ar². Similarly, the group L² is depicted by the combination of group or groups interspacing or linking the groups Ar³ and the basic group. The basic group of the compounds of the following schemes in general mean the group —N(R¹R²) unless otherwise indicated. Examples of the General Method 1 are a Displacement Process (Scheme 1a) and a Reductive Amination Process (Scheme 1b).

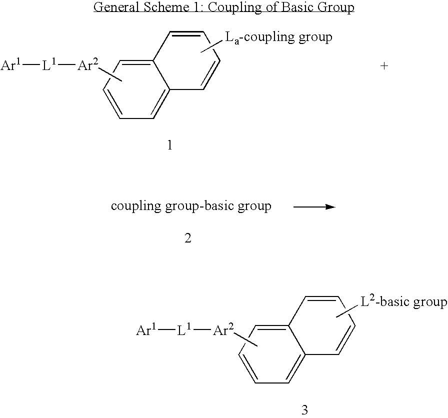

As outlined in Scheme 1a below, the coupling process of General Method 1 may consist of a displacement process whereby nucleophilic displacement of a leaving group, such as, but not limited to, halogen, triflate, tosylate, brosylate, mesylate, nosylate, nonaflate, tresylate, and the like, of Formula 4, by a nucleophilic basic group of Formula 5 affords the compounds of the invention. A leaving group is defined in one or more of the general reference texts described previously.

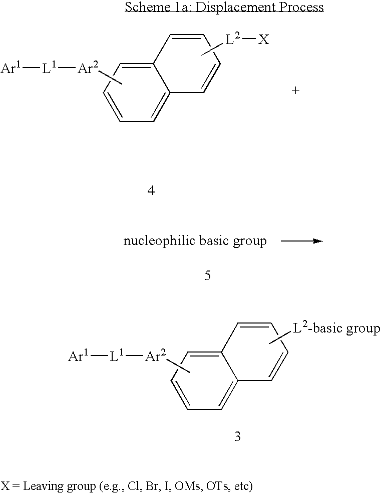

One to five equivalents of the nucleophilic basic group of Formula 5 and one to five equivalents of the reactive derivative of Formula 4 may be reacted in the presence, or absence, of an inert solvent. If necessary, the reaction may be carried out in the presence of a catalytic quantity to about five equivalents of a non-interfering base. A non-interfering interfering base is a base suitable for the intended reaction by virtue of the base not deleteriously affecting the reaction. One to two equivalents of base is normally preferred. The reaction is normally carried out between 0° C. and 120° C. Reaction time is normally 4 to 24 hours.

Nucleophilic basic groups would include, but would not be limited to ammonia, primary and secondary amines, guanidines, and the like. Specific nucleophilic basic groups include ammonia, methylamnine, dimethylamine, diethylamine, diisopropylamine, pyrrolidine, piperidine, morpholine, azetidine, thiomorpholine, piperazine, imidazole, and the like. Among the above nucleophilic basic groups dimethylamine, pyrrolidine, and piperidine are preferable.

If necessary, the reaction can be carried out with nucleophilic basic group synthon, i.e., a group that could readily be converted to a basic group by methods known to one skilled in the art. Nucleophilic basic group synthons would include, but would not be limited to, azide, phthalimide, protected amines, hexamethylenetetramine, cyanamide, cyanide anion, and the like. Following the displacement reaction, these groups would then be unmasked under standard conditions to afford the basic group. For example, displacement with potassium phthalimide followed by removal of the phthalimide group to afford the primary amine as in the Gabriel synthesis (see, March's Advanced Organic Chemistry, Reactions Mechanisms, and Structure, 5^{th} Edition, Michael B. Smith and Jerry March, Wiley Interscience, 2001, Chapter 10, and references cited therein). Application of the synthon equivalent to the basic group applies to the processes described in all of the General Methods 1 through 5.

Examples of “inert solvent” includes amide solvents (preferably DMF or DMAC), sulfoxide solvents (preferably DMSO), sulfone solvents (preferably sulfolane or dimethylsulfone), nitrile solvents (preferably acetonitrile), halogenated hydrocarbon solvents (preferably dichloromethane), aromatic solvents (preferably toluene or benzene), ether solvents (preferably diethylether or THF), ketone solvents (preferably acetone), ester solvents (preferably ethyl acetate), alcohol solvent (preferably MeOH or EtOH), etc. Two or more of the solvents can be mixed in an appropriate ratio for use. Among the above solvents, DMF and DMSO are preferable.

Examples of “base” include, for instance, hydrides of alkali metals and alkaline earth metals (e. g., lithium hydride, sodium hydride, potassium hydride, and the like), amides of alkali metals and alkaline earth metals (e. g., sodium amide, lithium diisopropyl amide, lithium hexamethyldisilazide, and the like), alkoxides (e. g. sodium methoxide, sodium ethoxide, potassium t-butoxide, and the like), inorganic bases, such as hydroxides of alkali metals or alkaline earth metals (e. g., sodium hydroxide, lithium hydroxide, potassium hydroxide, and the like), carbonates and hydrogen carbonates of alkali metals or alkaline earth metals (e. g., potassium carbonate, sodium bicarbonate, sodium carbonate, cesium carbonate, and the like), amine bases (such as, N-methylmorpholine, DBU, DBN, pyridine, 2,6-lutidine, triethylamine, diisopropylethylamine, and the like). Among the above bases, sodium hydride, potassium carbonate, and cesium carbonate are preferable.

As outlined in Scheme 1b below, the coupling process can consist of a Reductive Amination Process. A compound of Formula 6 is condensed with ammonia, or a primary, or secondary amine under dehydration/reduction conditions. Scheme 1b is a process analogous to that described in for example, Chem Pharm Bull 1999, 47 (8), 1154-1156; Synlett 1999, (11), 1781-1783; and J Med Chem 1999, 42 (26), 5402-5414 and references cited therein.

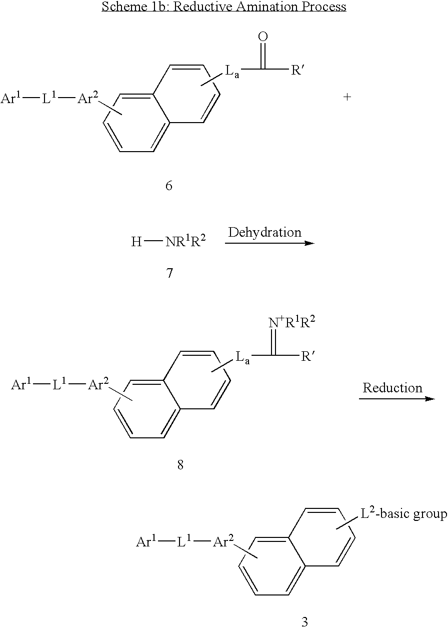

The carbonyl compound of Formula 6 is reacted with an amine of Formula 7 in an inert solvent under conditions that form the iminium species of Formula 8. The iminium species is reduced in-situ to form the compounds of Formula 3. The reaction is normally done in the presence of a dehydrating agent and a reducing agent. Amines of Formula 7 include, but are not be limited to ammonia, primary and secondary amines, and the like. Specific amine groups include ammonia, methylamine, dimethylamine, diethylarnine, diisopropylamine, pyrrolidine, piperidine, morpholine, azetidine, thiomorpholine, piperazine, imidazole, and the like. One to five equivalents of the amine group of Formula 7 and one to five equivalents of the reactive derivative of Formula 6 are reacted in the presence, or absence, of an inert solvent. The use of an excess of dehydrating agent is normally preferable. The reaction is carried out in the presence of one to hundred equivalents of a reducing agent. One to three equivalents of reducing agent is preferable. The reaction is normally carried out between 0° C. and 120° C. Reaction time is normally 4 to 24 hours. For the above amination reaction, MeOH and EtOH are preferable as inert solvents.

Examples of “dehydrating agents” may be anhydrous molecular sieves beads, anhydrous molecular sieve pellets, powdered anhydrous molecular sieves, anhydrous molecular sieves on supports (such as zeolite), anhydrous magnesium sulfate, anhydrous sodium sulfate, and the like. Among the above dehydrating agents, anhydrous molecular sieves pellets and powdered anhydrous molecular sieves are preferable.

Examples of “reducing agents” include hydrogen gas or hydrogen gas precursor and a hydrogenation catalyst. Other “reducing agents” include sodium cyanoborohydride, sodium triacetoxyborohydride, sodium borohydride, sodium borohydride/Ti (Oi-Pr)4, borohydride-exchange resin, and the like. Examples of “hydrogen gas precursors” include formic acid, 1,4-cyclohexadiene, and the like. Examples of “hydrogenation catalyst” include palladium on carbon, platinum on carbon, rhodium, ruthenium, nickel and the like. The metal can be used as a finely dispersed solid or absorbed on a support, such as carbon or alumina. Among the above reducing agents, sodium cyanoborohydride and sodium triacetoxyborohydride are preferred.

### General Method 2: Coupling of the linker group

The compounds of Formula 3 can be prepared by the General Method 2, described in General Scheme 2, via reaction of the coupling group of Formula 9 with a coupling group of Formula 10.

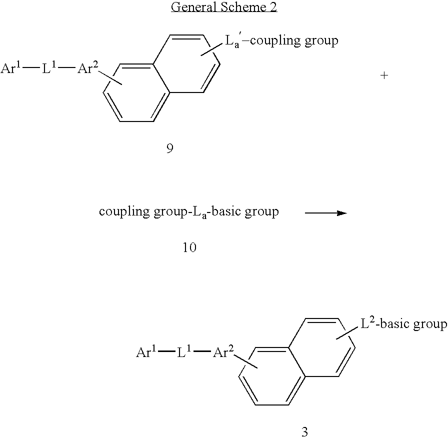

Examples of the General Method 2 are an Ether/Thioether Alkylation Process (Scheme 2a), an Acylation/Sulfonylation Process (Scheme 2b), Urea/Thiourea/Guanidine Coupling Process (Scheme 2c1, 2c2, 2c3), an Organometallic Process (Scheme 2d), and a Wittig-type Coupling (Scheme 2e). As outlined in Scheme 2a below, the coupling process of General Method 2 can consist of a Ether/Thioether Alkylation Process. Nucleophilic displacement by an alcohol or thiol-containing compound of Formula 11 (or Formula 11′) with a compound of Formula 12 (or Formula 12′) containing a leaving group affords the ether and thioether compounds of Formula 13. Scheme 2a is a process analogous to that described in The Chemistry of the Ether Linkage; Patai, Wiley, 1967, 446, 460; and in March's Advanced Organic Chemistry, Reactions Mechanisms, and Structure, 5^{th} Edition, Michael B. Smith and Jerry March, Wiley Interscience, 2001, Chapter 10.

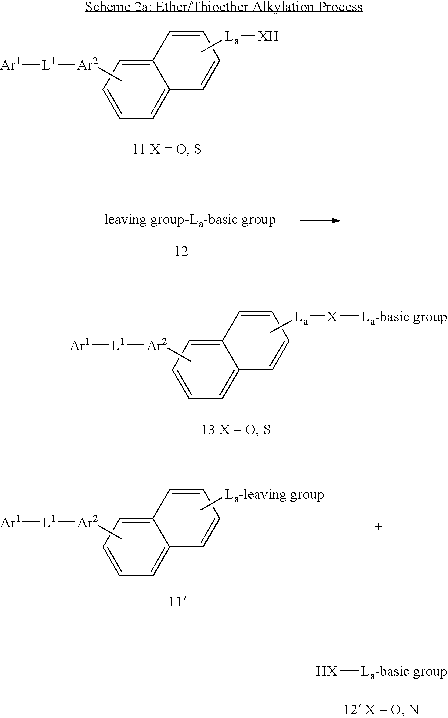

One to five equivalents of the alcohol or thiol of Formula 11 (or Formula 11′) and one to five equivalents of the reactive derivative of Formula 12 (or Formula 12′) are reacted in the presence, or absence, of an inert solvent. If necessary, the reaction can be carried out in the presence of a catalytic quantity to ten equivalents of a non-interfering base. One to three equivalents of base is normally preferable. The reaction is typically carried out between 0° C. and 120° C. Reaction time is typically 4 to 24 hours, but may be longer depending on the particular substrate. Preferred bases for the above reaction include sodium hydride, potassium carbonate and cesium carbonate. If necessary, the reaction may be carried out with basic group synthon incorporated as the basic group in Formula 12, i.e., a group that could readily be converted to a basic group by methods known to one skilled in the art. Basic group synthons would include, but not be limited to, halogen, protected amine, nitrile, aldehyde, and the like. Following the ether/thioether alkylation reaction, these groups would then be unmasked or converted under standard conditions to afford the basic group. For example, alkylation with 1-iodo4-chloro-butane would give a 4-chlorobutane derivative of compound 11. The chloride could then be converted by the Displacement Process, described above in Scheme 1a, into the basic group of a compound of Formula 13. Among the inert solvents, DMF and DMSO are preferable.

As outlined in Scheme 2b below, the coupling process of General Method 2 can consist of an Acylation/Sulfonylation Process. Acylation or sulfonylation of an alcohol or amine compound of Formula 14 with a carboxylic acid or sulfonic acid compound of Formula 15 affords the ester, amide, sulfonic ester, or sulfonamide compounds of Formula 16. Alternatively, acylation or sulfonylation of an alcohol or amine compound of Formula 18 with a carboxylic acid or sulfonic acid compound of Formula 17 affords the ester, amide, sulfonic ester, or sulfonamide compounds of Formula 19. If necessary, the reaction can be carried out with a basic group synthon incorporated as the basic group in Formula 15 or Formula 18, i.e., a group that could readily be converted to a basic group by methods known to one skilled in the art. Basic group synthons would include, but not be limited to, halogen, protected amine, nitrile, aldehyde, and the like. Following the Acylation/Sulfonylation reaction, these groups would then be unmasked or converted under standard conditions to afford the basic group.

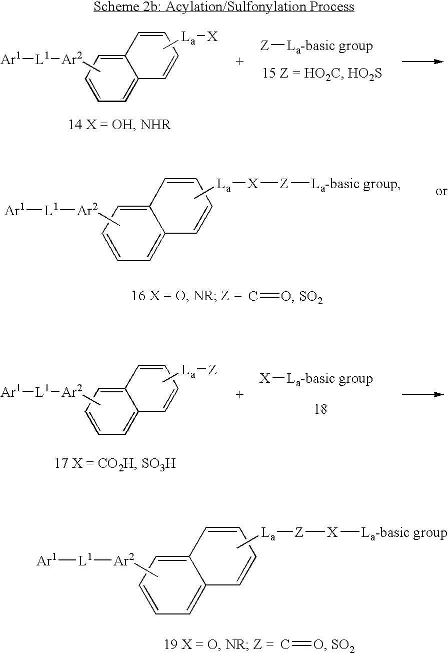

The carboxylic acid (or sulfonic acid) residue of compound 15 (or compound 17) is activated for coupling as a “reactive acylating agent.” “Reactive acylating agents” are described in detail in Advanced Organic Chemistry, 4^{th} Edition, Part B, Reactions and Synthesis, Francis A. Carey and Richard J. Sundberg, Kluwer Academic/Plenum Publishers, 2000, Chapter 3, and references cited therein. The “reactive acylating agent” can be formed and isolated, then reacted with the compound of Formula 14 (or 18), or formed in situ and reacted with the compound of Formula 14 (or 18), to form the compound of Formula 16 (or 19).

One to five equivalents of the “reactive acylating agent” of compound 15 (or compound 17) and one to five equivalents of compound of Formula 14 (or 18) are reacted in an inert solvent. If necessary the reaction may be carried out in the presence of one to five equivalents of 1-hydroxybenzotriazole, 1-hydroxy-7-azabenzotriazole, and (or) a catalytic quantity to five equivalents of a base. The reaction is normally carried out between 0° C. and 120° C. Reaction time is normally 4 to 48 hours.

Examples of “reactive acylating agent” of compound 15 (or compound 17) include acid halides (e.g., acid chloride, acid bromide, and the like), mixed acid anhydrides (e. g., acid anhydrides with C₁-C₆ alkyl-carboxylic acid, C₆-C₁₀ aryl-carboxylic acid, and the like), activated esters (e. g., esters with phenol which may have substituents, 1-hydroxybenzotriazole, N-hydroxysuccinimide, 11-hydroxy-7-azabenzotriazole, and the like), thioesters (such as, 2-pyridinethiol, 2-imidazolethiol, and the like), N-acylimidazoles (e.g., imidazole, and the like), etc.

A “reactive acylation agent” may also be formed reacting the carboxylic acid (or sulfonic acid) residue of compound 15 (or compound 17) with a dehydration/condensation agent. Examples of a “dehydration/condensation agent” include dicyclohexylcarbodimide (DCC), 1-ethyl-3-(3-dimethylaminopropyl)carbodimide (EDCI), (2-ethoxy-1-ethoxycarbonyl-1,2-dihydroquinoline (EEDQ), and the like. Preferred solvents for the above reaction include acetonitrile, THF, and dichloromethane.

Preferred bases for the above reaction include triethylamine, pyridine, and dimethylaminopyridine are preferable.

As outlined in Scheme 2c2, Scheme 2c2, and Scheme 2c3 below, the coupling process of General Method 2 can consist of a Urea/Thiourea/Guanidine/Carbamate-Type Coupling Process. The processes described are analogous to that described in U.S. Pat. Nos. 5,849,769 and 5,593,993, and references cited therein.

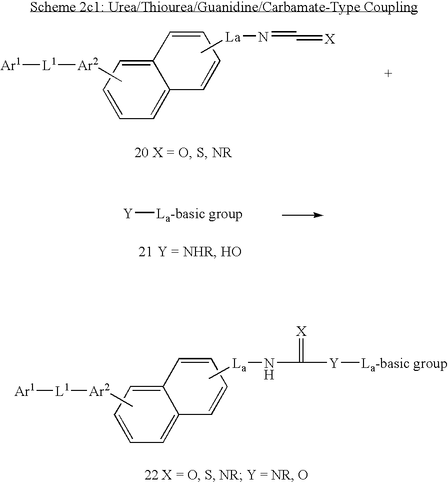

One to five equivalents of the isocyanate, isothiocyanate, carbodiimide of Formula 20 and one to five equivalents of compound of Formula 21 are reacted in an inert solvent. The reaction is typically carried out between 0° C. and 150° C. Preferred reaction time is between 4 to 48 hours. Preferred solvents for the above reaction include acetonitrile, DMF, DMSO, THF, and dichloromethane.

If necessary, the reaction can be carried out with a basic group synthon incorporated as the basic group wherein a synthon is as described ealier. Following the Urea/Thiourea/Guanidine/Carbamate-Type Coupling Process, these groups would then be unmasked or converted under standard conditions to afford the basic group.

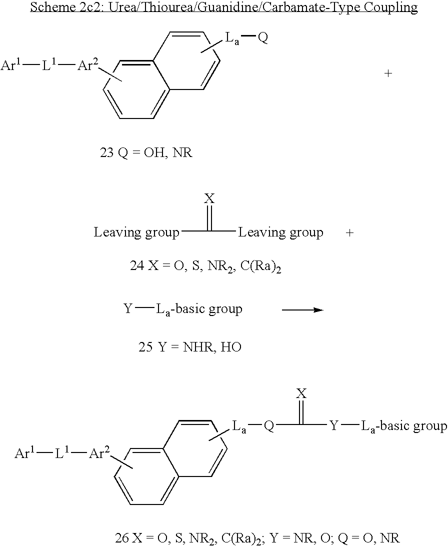

Approximately one equivalent of the compound of Formula 23 and one equivalent of compound of Formula 24 and one equivalent of the compound of Formula 25 are reacted in an inert solvent. The reaction is typically carried out between 0° C. and 150° C. Reaction time is normally 4 to 48 hours. The sequence of addition depends upon the reactivity of the individual reagents. The intermediate addition product may be isolated and subsequently be condensed with the second reagent. The reaction may or may not require the addition of a catalyst. Preferred solvents for the above reaction include acetonitrile, DMF, DMSO, THF, toluene, isopropanol, and dichloromethane. Acids and bases as described previously may be used to catalyze the above reaction.

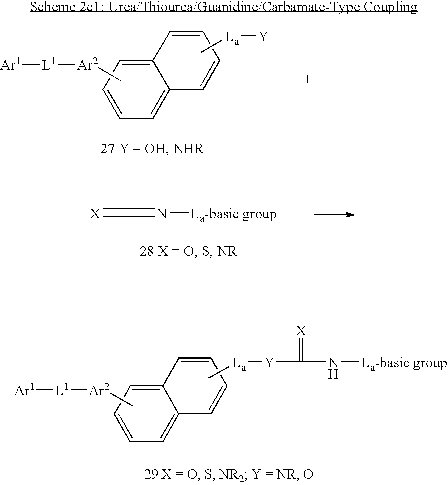

One to five equivalents of the isocyanate, isothiocyanate, carbodiimide of Formula 28 and one to five equivalents of compound of Formula 27 are reacted in an inert solvent. The reaction is normally carried out between 0° C. and 150° C. Reaction time is normally 4 to 48 hours.

As outlined in Schemes 2d below, the coupling process of General Method 2 may consist of an Organometallic Coupling Process.

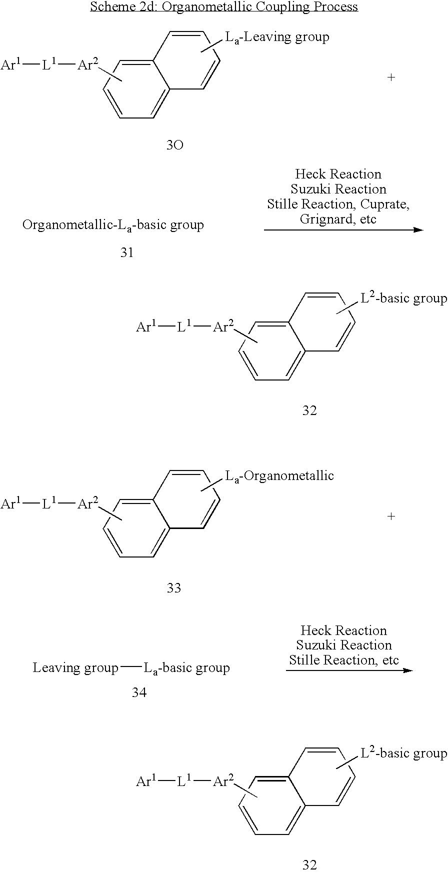

The compound of Formula 30 (or Formula 34) is coupled with an organometallic compound of Formula 31 (or Formula 33) (containing a basic group, or basic group precursor) in an Organometallic Coupling Process to afford the compounds of the invention of Formula 32.

“Organometallic Coupling Processes” include “palladium-catalyzed cross coupling reactions,” such as, Heck-type coupling reactions, Suzuki-type coupling reactions and Stille-type coupling reactions. Other organometallic coupling reactions include, organocuprate coupling reactions, Grignard coupling reactions, and the like. A general description of Organometallic Coupling is given in detail in Advanced Organic Chemistry, 4^{th} Edition, Part B, Reactions and Synthesis, Francis A. Carey and Richard J. Sundberg, Kluwer Academic/Plenum Publishers, 2000, Chapters 7 and 8, and references cited therein.

In Scheme 2d. the compound of Formula 30 (or Formula 34) is coupled with the organometallic reagent of Formula 31 (or Formula 33) in the presence, or absence, of a transition metal catalyst, and/or a phosphine or arsine, and/or a base in an inert solvent. Other additives, such as, copper salts, silver salts, and the like may be added. Approximately one equivalent of the compound of Formula 30 (or Formula 34) is reacted with one to five equivalents of the compound of Formula 31 (or Formula 33) with the appropriate additives in an inert solvent. The reaction is normally carried out between −78° C. and 200° C. for between 4 to 72 hours.

Examples of “organometallic reagents” include, organomagnesium, organozinc, mixed organocuprate, organostannane, or organoboron compounds, and the like. Examples of “transition metal catalysts” include, palladium and nickel catalysts, such as, Pd(OAc)₂, Pd (PPh₃)₄, PdCl₂, Pd(PPh₃)Cl₂, Pd(OCOCF₃)₂, (CH₃C₄H₅P)₂PdCl₂, [(CH₃CH₂)₃P]₂PdCl₂, [(C₆H₁₁)₃P]₂PdCl₂, [(C₆H₅) ₃P]₂PdBr₂, Ni(PPh₃)₄, (C₆H₄CH═CHCOCH═CHC₆H₅)₃Pd, and the like. Among the above transition metal catalysts, Pd(OAc)₂, Ni(PPh₃)₄, and Pd(PPh₃)₄ are preferable.

Examples of “phosphines or arsines” include, a trialkyl or triarylphosphine or arsine, such as triisopropylphosphine, triethylphosphine, tricyclopentylphosphine, triphenylphosphine, triphenylarsine, 2-furylphosphine, tri-o-tolylphosphine, tricyclohexylphosphine, 1,2-bis(diphenylphosphino)ethane, 1,3-bis(diphenylphosphino)propane, 1,4-bis(diphenylphosphino)butane, 2-(Di-t-butylphosphino)biphenyl, and the like. Among the above “phosphines and arsines,” tri-o-tolylphosphine, triphenylarsine, and tricyclohexylphosphine are preferable.

Examples of “other additives” include, copper salts, zinc salts, lithium salts, ammonium salts and the like. Among the above “other additives,” CuI, LiCl, and n-Bu₄N⁺Cl³¹ are preferable. If necessary, the reaction can be carried out with a basic group synthon incorporated as the basic group as described previously. As outlined in Schemes 2e below, the coupling process of General Method 2 can consist of a Wittig-type Coupling Process. The compound of Formula 33 (or Formula 37) is coupled with the phosphorus ylene (or ylide) reagent of Formula 34 (or Formula 36) to afford the compounds of Formula 35 of the invention. A general description of Wittig-type Coupling Reactions is given in detail in general reference texts such as Advanced Organic Chemistry, 4^{th} Edition, Part B, Reactions and Synthesis, Francis A. Carey and Richard J. Sundberg, Kluwer Academic/Plenum Publishers, 2000, Chapter 2, and references cited therein.

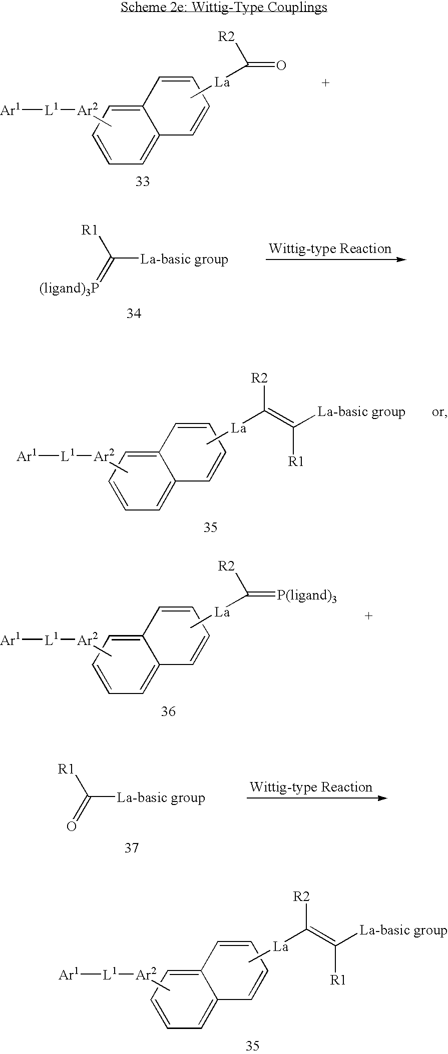

The compound of Formula 33 (or Formula 37) is coupled with the phosphorus ylene (or ylide) reagent of Formula 34 (or Formula 36) in the presence, or absence, a base in an inert solvent to form the compounds of the invention of Formula 35. Other additives, such as, lithium salts, sodium salts, potassium salts, and the like may be added. Approximately one to five equivalents of the compound of Formula 33 (or Formula 37) is reacted with one to five equivalents of the compound of Formula 34 (or Formula 36) with the appropriate additives an inert solvent. The reaction is normally carried out between −78° C. and 120° C. for between 2 to 72 hours. The Wittig reaction product may be reduced to form other compounds of the invention using reducing agents known to one of skill in the art and/or described previously. Preferred bases for the above organometallic reactions include, sodium hydride, DBU, potassium t-butoxide, and lithium hexamethyldisilazide.

### General Method 3: Coupling of the Five-Membered Ring

### Heterocycle and Phenyl Groups

The compounds of Formula 3 can be prepared by the General Method 3, described in General Scheme 3, via coupling of the compounds of Formula 38 with a compound of Formula 39. An example of the General Method 3 is an Aryl Coupling Process (Scheme 3a). The aryl-coupling reaction is carried out in accordance with per se known methods, or analogous methods thereto, such as those described in the general reference texts discussed previously.

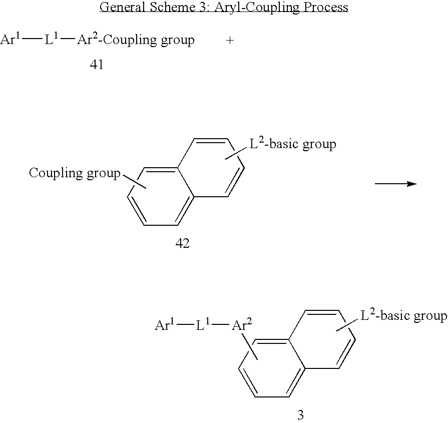

The compound of Formula 44 (or Formula 45) is coupled with an organometallic compound of Formula 43 (or Formula 46) in an Aryl Coupling Process to afford the compounds of the invention of Formula 3.

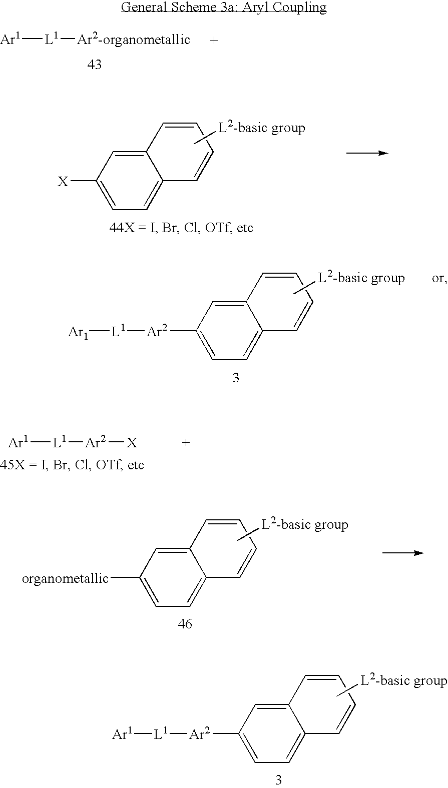

The compound of Formula 44 (or Formula 45) is coupled with the organometallic reagent of Formula 43 (Formula 46) in the presence, or absence, of a transition metal catalyst, and (or) a phosphine or arsine, and (or) a base in an inert solvent. Other additives, such as, copper salts, silver salts, and the like may be added. Approximately one equivalent of the compound of Formula 44 (or Formula 45) is reacted with one to five equivalents of the compound of Formula 43 (Formula 46) with the appropriate additives an inert solvent. The reaction is normally carried out between −78° C. and 200° C. for between 4 to 72 hours. Examples of “organometallic reagents”, “transition metal catalysts” “phosphines or arsines” “other additives” and “base” have been described previously.

### General Method 4: Heterocycle Formation

The compounds of Formula 3 can be prepared by the General Method 4, described in General Scheme 4, via reaction of the compound of Formula 47 containing a coupling group with a compound of Formula 48 containing a coupling group, where during the course of the coupling reaction the coupling groups form the 5-membered ring heterocycle between the linker L¹ and the phenyl ring. Ar¹, L¹, Ar², L², and basic group are defined as above. Examples of heterocyclic ring forming reactions are given in Comprehensive Heterocyclic Chemistry, Volumes 1-8, A. P. Katritzky and C. W. Rees Eds, Pergamon Press, 1984; Heterocyclic Chemistry, 3^{rd} Ed, Thomas L. Gilchrist, Addison-Wesley-Longman Ltd, 1997; An Introduction to the Chemistry of Heterocyclic Compounds, 3^{rd} Ed, R. M. Acheson, Wiley Interscience, 1976; etc, and references cited therein. Specific examples of the General Method 4 include an Oxadiazole Process (Schemes 4a and 4b), a Thiadiazole Process (Scheme 4c), and an Oxazole Process (Scheme 6 a-e). If necessary, the reaction can be carried out with a basic group synthon incorporated as the basic group, i.e., a group that could readily be converted to a basic group by methods known to one skilled in the art. Basic. group synthons would include, but not be limited to, halogen, protected amine, nitrile, aldehyde, and the like. Following the Heterocycle Formation Process, these groups would then be unmasked or converted under standard conditions to afford the basic group.

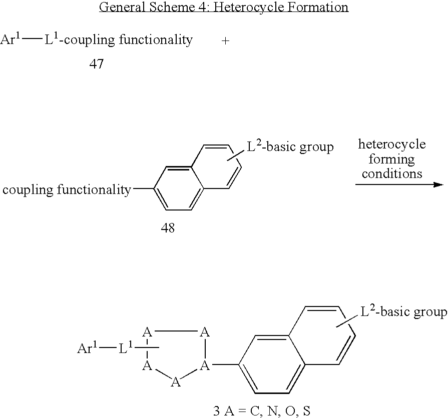

As outlined in Scheme 4a below, the coupling process of General Method 4 can consist of a Oxadiazole Process. The diacylhydrazide compound of Formula 51 is produced by acylation of an acylhydrazide of Formula 50 (or Formula 53) by a carboxylic acid derivative of Formula 49 (or Formula 54). The acylation process is carried out in accordance with the above Acylation/Sulfonylation Process of the General Method 2. The diacylhydrazide is cyclized to the oxadiazole compounds of the invention of Formula 52 utilizing dehydration processes analogous to that described in J Org Chem 1999, 64 (19), 6989-6992; and Chem Heterocycl Compd 1999, 35 (3), 275-280.

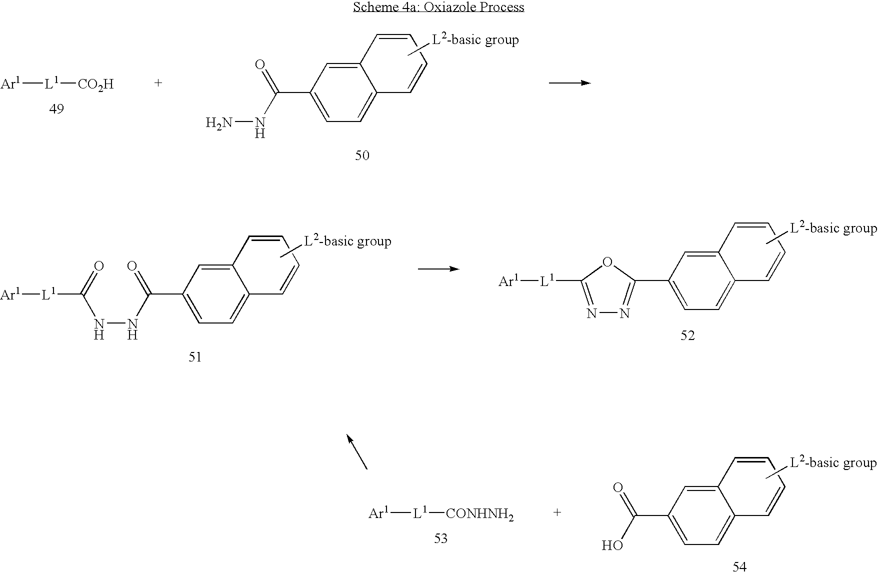

One equivalent of compound of Formula 51 is reacted with one to equivalents of a dehydrating agent in the presence, or absence, a base in an inert solvent. The reaction is normally carried out between 25° C. and 250° C. for between 4 to 72 hours. Examples of “dehydrating agents” include, SOCl₂, H₃PO₄, POCl₃, PCl₅, Tf₂O, Ac₂O, PPh₃—I₂, PPh₃—Br₂, PPh₃—Cl₂, PPh₃—CBr₄, PPh₃—CCl₄, PPA, NH(Tms)₂, P₂O₅, Me₂SiCl₂, PhOPCl₂, H₂SO₄, and the like.

As outlined in Scheme 4b below, an alternative Oxadiazole Process may be utilized to prepare the oxadiazole compounds of the invention of Formula 52. The carboxylic acid derivative of Formula 49 (or 54) is activated for coupling as a “reactive acylating agent.” The acylation process is carried out in accordance with the above Acylation/Sulfonylation Process of the General Method 2. The acylated intermediate is converted to the oxadiazole compounds of the invention of Formula 52. The process is analogous to that described in Synth Commun 1994, 24 (11), 1575-1582; J Org Chem 1961, 26, 2372; Synthetic Commun 24 (11), 1575-1582 (1994); etc, and references cited therein.

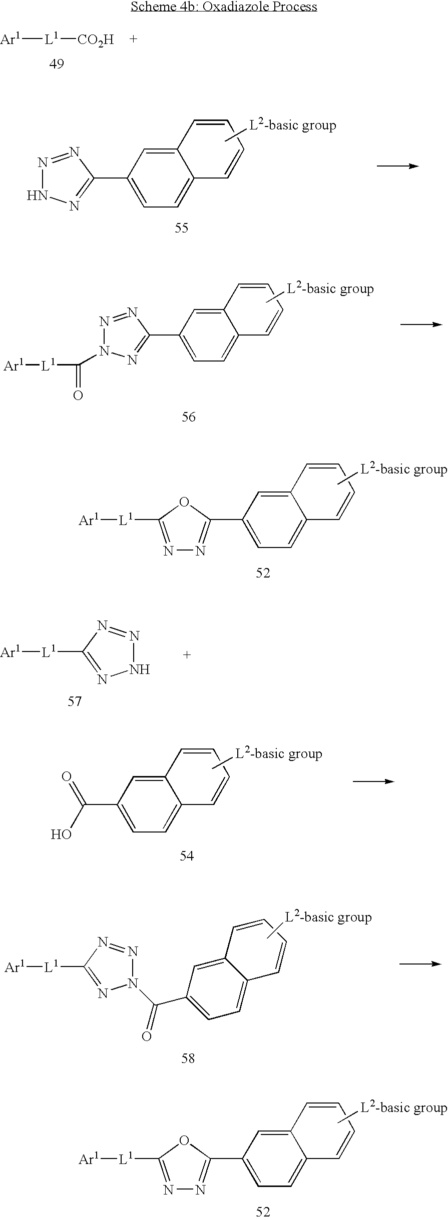

One to five equivalents of the “reactive acylating agent” of compound 49 (or compound 54) and one to five equivalents of compound of Formula 55 (or 57) are reacted in an inert solvent. If necessary the reaction can be carried out in the presence of a one to five equivalents of 1-hydroxybenzotriazole, 1-hydroxy-7-azabenzotriazole, and (or) a catalytic quantity to five equivalents of a base. The reaction intermediate of Formula 56 (or 58) may, or may not, be isolated. The reaction is normally carried out between 0° C. and 200° C. Reaction time is normally 4 to 48 hours. Reactive acylation agents have been discussed and may similarly be prepared for compounds 49 and/or 55 as described previously.

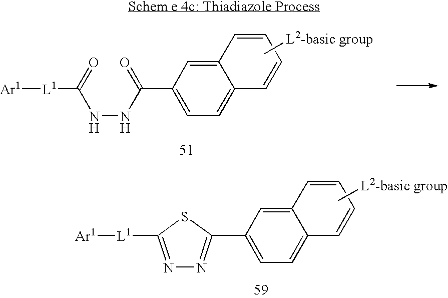

One equivalent of compound of Formula 51 is reacted with one to five equivalents of a thiol dehydrating agent in the presence, or absence, a base in an inert solvent. The reaction is normally carried out between 25° C. and 250° C. for between 4 to 72 hours. Examples of “thiol dehydrating agents” include, P₂S₅, Lawesson reagent, and the like.

### General Method 5: Coupling of the Linker Group L¹

The compounds of Formula 3 can be prepared by the General Method 5, described in General Scheme 5, via reaction of the coupling group of Formula 62 with a coupling group of Formula 63, where during the course of the coupling reaction the coupling groups are retained, or lost, to form the linker L¹ between the 5-membered ring heterocyclic group and Ar¹. Ar¹, L¹, Ar², L², and basic group are defined as above. La is defined as a group that when the coupling process occurs results in the formation of the linker L² defined above. Examples of the General Method 5 are an Ether/Thioether Alkylation Process (Scheme 5a), an Acylation/Sulfonylation Process Process (Scheme 5b), an Urea/Thiourea/Guanadine Coupling Process (Scheme 5c1, 5c2, 5c3), an Organometallic Process (Scheme 5d), and a Wittig-type Coupling (Scheme 5e).

If necessary, the reactions below may be carried out with a basic group synthon incorporated as the basic group, as described previously. Following the Coupling of the Linker Group (L¹) Process, these groups would then be unmasked or converted under standard conditions to afford the basic group.

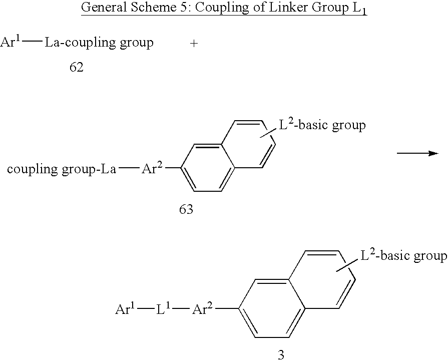

As outlined in Scheme 5a below, the coupling process of General Method 5 can consist of a Ether/Thioether Alkylation Process. Nucleophilic displacement by an alcohol or thiol-containing compound of Formula 64 (or Formula 68) with a compound of Formula 65 (or Formula 67) containing a leaving group affords the ether and thioether compounds of Formula 66 of the invention. The processes are analogous to the process described for the General Method 2, described in Scheme 2a, and carried out in accordance with the above method.

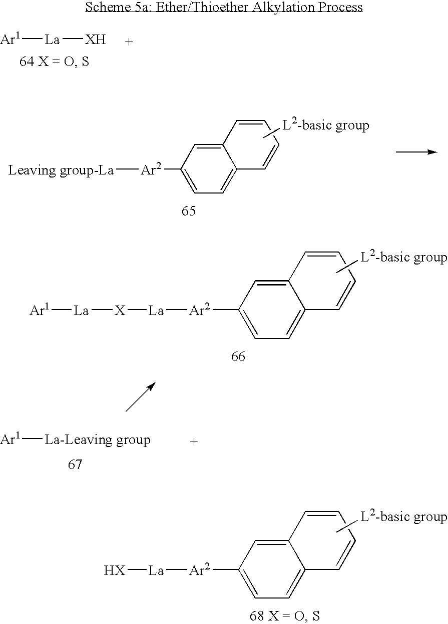

As outlined in Scheme 5b below, the coupling process of General Method 5 can consist of an Acylation/Sulfonylation Process. Acylation or sulfonylation of an alcohol or amine compound of Formula 70 with a carboxylic acid or sulfonic acid compound of Formula 69 affords the ester, amide, sulfonic ester, or sulfonamide compounds of Formula 71. Alternatively, acylation or sulfonylation of an alcohol or amine compound of Formula 72 with a carboxylic acid or sulfonic acid compound of Formula 73 affords the ester, amide, sulfonic ester, or sulfonamide compounds of Formula 74.

The processes are analogous to the process described for the General Method 2, described in Scheme 2b, is carried out in accordance with the above method.

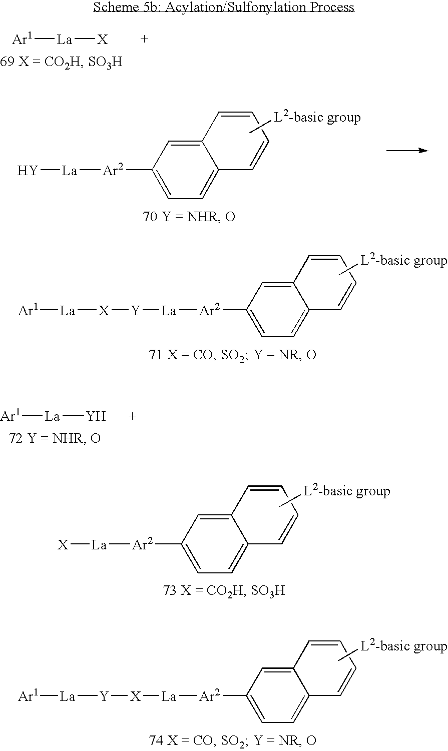

As outlined in Schemes 5c1, 5c2, and 5c3, below, the coupling process of General Method 5 can consist of a Urea/Thiourea/Guanidine/Carbamate-Type Coupling Process to afford the compounds of Formula 77, 81, and 84 of the invention. The processes are analogous to the processes described for the General Method 2, described in Schemes 2c1, 2c2, and 2c3, are carried out in accordance with the above method.

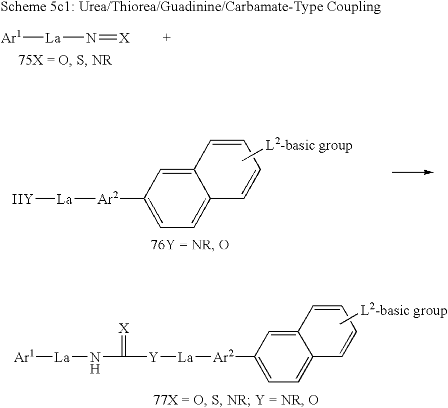

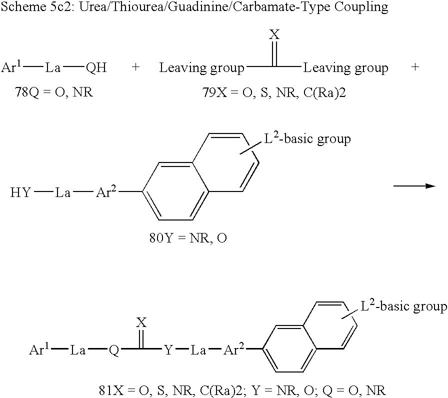

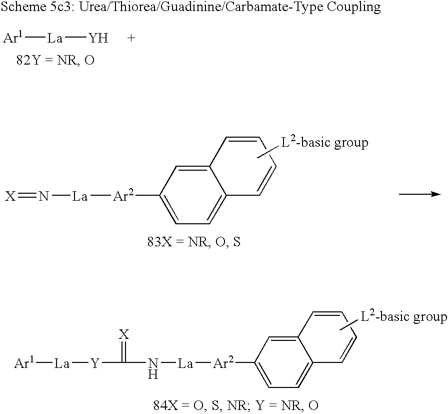

As outlined in Schemes 5d below, the coupling process of General Method 5 can consist of a Organometallic Coupling Process. The compound of Formula 86 (or Formula 87) is coupled with an organometallic compound of Formula 85 (or Formula 88) in an Organometallic Coupling Process to afford the compounds of Formula 3 of the invention. The processes are analogous to the processes described for the General Method 2, described in Scheme 2d, and are carried out in accordance with the above methods.

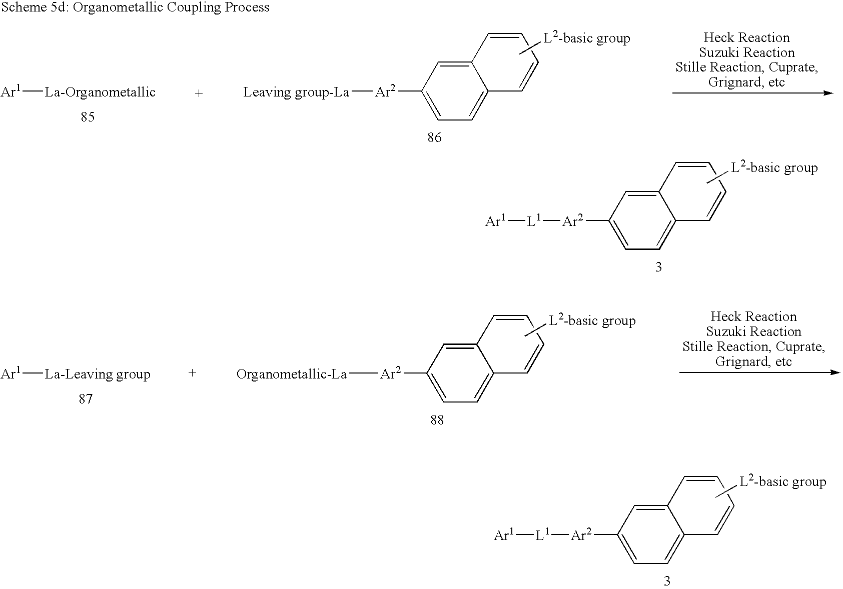

As outlined in Schemes 5e below, the coupling process of General Method 2 can consist of a Wittig-type Coupling Process. The compound of Formula 89 (or Formula 93) is coupled with the phosphorus ylene (or ylide) reagent of Formula 90 (Formula 92) to afford the compounds of Formula 91 of the invention. The processes are analogous to the processes described for the General Method 2, described in Scheme 2e, and are carried out in accordance with the above methods.

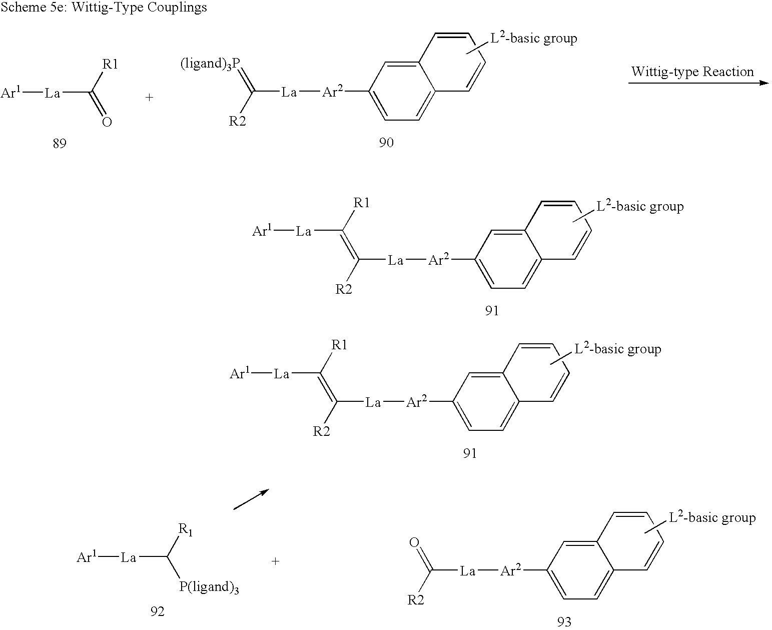

### Preparation of Oxazole and Oxathiazole Compounds

As outlined in schemes 6a-c (below) the formation of oxazoles and thiazoles require elevated temperatures from 80-120° C. in solvents like dimethylformamide (scheme 6a+b) or phosphoryl chloride (scheme 6c).

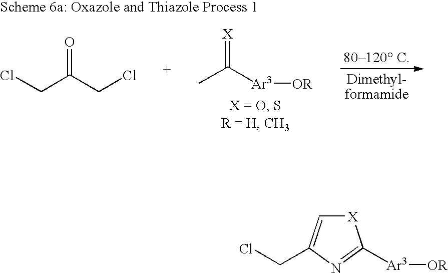

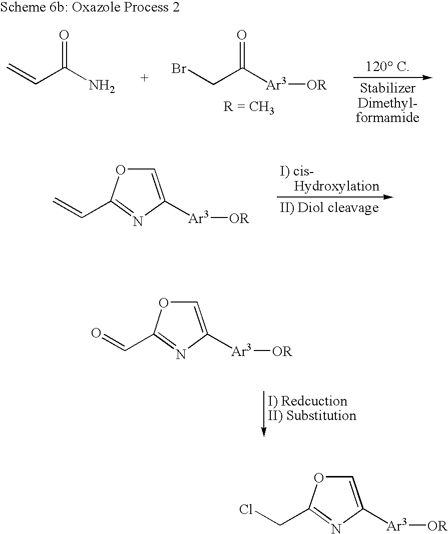

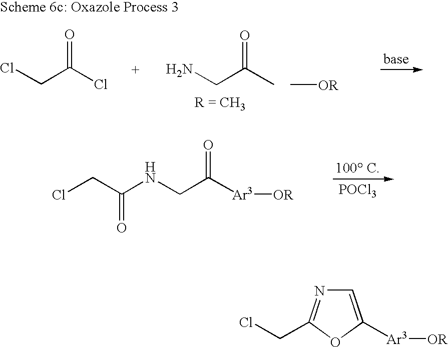

These heterocyclic cyclisations result either in chloromethyl substituted oxazoles and thiazoles (scheme 6 a+c) or in vinyl substituted oxazole (scheme 6b). After cis-hydroxylation of the later vinyl substituted oxazole, followed by diol cleavage, as known to the artesian, the resulting formyl substituted oxazole can be converted via reduction and substitution to the chloro methyl substituted oxazole (scheme 6b). The cyclisation of the α-bromoketone with acrylamide (scheme 6b) is preferably performed in the presence of a stabiliser (such as 2,6 di-tert.-butyl-4-methyl-phenol) to prevent polymerisation of the acrylamide. As outlined in scheme 6c, the condensation of 2-chloro acetyl chloride with an α-aminoketone in presence of a base such as, for example, triethylamine, affords a product in high yield that can be cyclised in phosphoryl chloride to result in formation of an oxazole. Unlike general scheme 4, these heterocyclic formations of oxazoles and thiazoles do not work as desired in the presence of Ar¹—L¹— nor in the presence of —L²— basic group, so that these groups have to be introduced later, as outlines in schemes 6d and 6e.

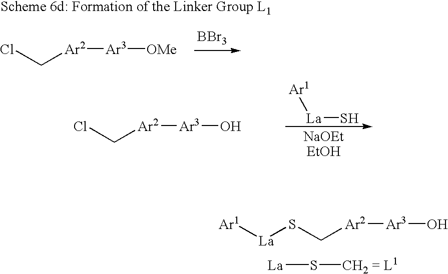

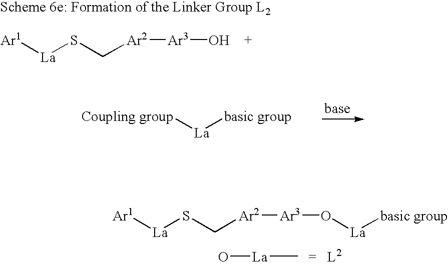

In order to achieve formation of the linker L¹, the chloromethyl substituted oxazoles or thiazoles from scheme 6a-c can be used as alkylation substrates for thiolates (scheme 6d). Therefore, a thiol is treated with a base, like sodium ethoxide in ethanol, before addition of the chloro methyl substituted oxazole. This alkylation proceeds in the presence of an unprotected phenol. The unprotected phenol can be incorporated into linker L² in a subsequent reaction, as outlined in scheme 6e in solvents such as dimethylformamide and involving bases such as potassium carbonate. As outlined in scheme 6d, the phenol may be obtained from the Lewis-acid mediated cleavage of a methylether with Lewis-acids, preferably, borontribromide in solvents such as dichloromethane.

For compounds wherein Ar² is oxazole, positional isomers of the oxazole group (e.g isoxazole) may be made as shown in Scheme 7.

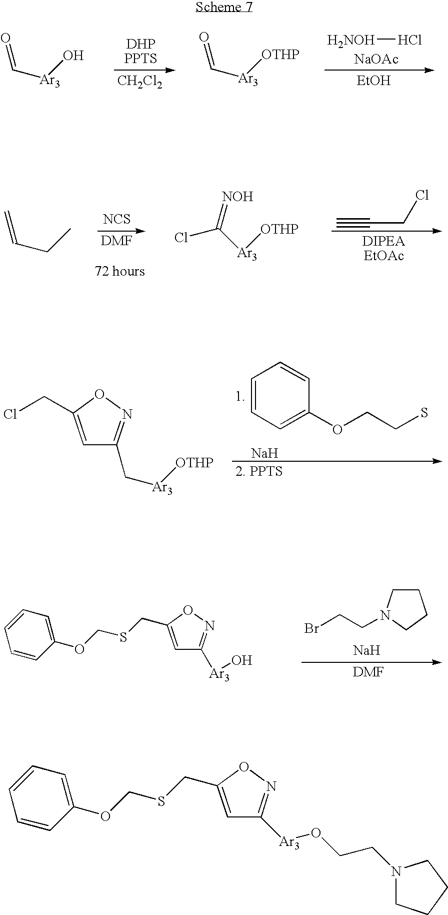

The hydroxyaldehyde is protected as the tetrahydropyran (THP) ether, using dihydropyran and p-toluenesulfonic acid (PPTS) in dichloromethane. The aldehyde functionality is converted to an oxime with hydroxylamine hydrochloride and sodium acetate in ethanol. The oxime is then converted to a chloro-oxime with NCS in DMF. Dipolar cycloaddition of the chloro-oxime and 3-chloropropyne in ethyl acetate using DIPEA as catalyst gives the intermediate chloromethylisoxaole. This is then used to alkylate 2-phenoxy-ethanethiol. This intermediate is deprotected with PPTS to give the phenol. The phenol is alkylated with 1-(2-chloro-ethyl)-pyrrolidine hydrochloride to give the final product.

The 1,2,4-oxadiazole isomer may be prepared following the procedure of Scheme as shown in Scheme 8 for the particular example.

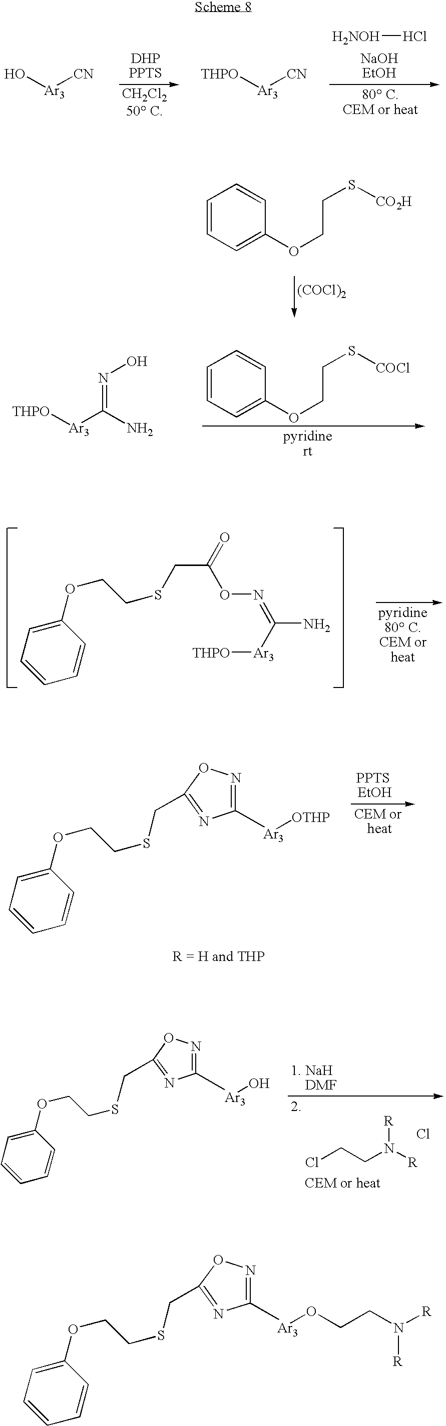

As shown, the cyanophenol is protected as the Tetrahydropyran (THP) ether using dihyropyran and dihydropyran and p-toluenesulfonic acid (PPTS) in dichloromethane. The cyano functionality is converted to an amidoxime functionality by reaction with hydroxylamine hydrochloride and NaOH in ethanol in a microwave chamber at 80 C. A mixture of the amidoxime and acid chloride in pyridine is microwaved at 80 C to give the isoxazole intermediate as a mixture of protected THP ether and deprotected phenol. After removal of pyridine under vacuum, the reaction products are treated with PPTS in ethanol and microwaved at 75 C to deprotect any remaining THP ether, giving the [1,2,4]oxadiazol-3-yl]-phenol. The phenol is alkylated with 1-(2-chloro-ethyl)-pyrrolidine hydrochloride to give the final product.

One of skill in the art is aware that other compounds within the scope of the invention may be made as shown or by modifications to the procedures provided which are not cumbersome and are known to one of skill in the art or accessible in the general reference texts or literature available to one of skill in the art. Futhermore, in addition to the discussive procedures herein, detailed examples are provided which would further assist one of skill in the art to make the appropriate modifications to arrive at compounds within the scope that are not specifically exemplified.

### Demonstration of Function

In order to demonstrate that compounds of the present invention have the capacity to bind to and inhibit the function of MCHR1, binding and functional assays were established. All ligands, radioligands, solvents and reagents employed in these assays are readily available from commercial sources or can be readily prepared by those skilled in the art.

The full-length cDNA for human MCHR1 was cloned from a human adult brain cDNA library (Edge Biosystems, Cat. 38356) by standard polymerase chain reaction (PCR) methodology employing the following primers: sense, 5′-GCCACCATGGACCT GGAAGCCTCGCTGC-3′; anti-sense, 5′-TGGTGCCCTGACTTGGAGGTGTGC-3′. The PCR reaction was performed in a final volume of 50 μl containing 5 μl of a 10× stock solution of PCR buffer, 1 μl of 10 mM dNTP mixture (200 μM final), 2 μl of 50 mM Mg(SO₄) (2 mM final), 0.5 μl of 20 μM solutions of each primer (0.2 μM final), 5 μl of template cDNA containing 0.5 ng DNA, 0.5 μl of Platinum Taq High Fidelity DNA polymerase (Gibco Life Technologies) and 36 μl of H₂O. PCR amplification was performed on a Perkin Elmer 9600 thermocycler. After denaturation for 90 sec at 94° C., the amplification sequence consisting of 94° C. for 25 sec, 55° C. for 25 sec and 72° C. for 2 min was repeated 30 times, followed by a final elongation step at 72° C. for 10 min. The desired PCR product (1.1 Kb) was confirmed by agarose gel electrophoresis and the band was extracted from the gel by Geneclean (Bio101) following the manufacturer's instructions. Following extraction, the cDNA fragment was cloned into pCR2.1-TOPO plasmid (Invitrogen) to confirm the identity and sequence.

In order to generate cell lines stably expressing MCHR1, the insert was then subcloned into the Xba I and Not I sites of pcDNA(+)-3.1-neomycin (Invitrogen). After purification by Qiagen Maxi-prep kit (QIAGEN, Inc.), the plasmid was transfected by Fugene 6 (Roche Applied Science) into AV12 cells that had been previously transfected with the promiscuous G protein G_{□15}. The transfected cells were selected by G418 (800 μg/ml) for. 10-14 days and single colonies were isolated from culture plates. The G418-resistant colonies were further selected for MCHR1 expression by measuring MCH-stimulated Ca²⁺ transients with a fluorometric imaging plate reader (FLIPR, Molecular Devices).

Typically, individual clones are plated out in 96-well plates at 60,000 cells per well in 100 μl of growth medium (Dulbecco's modified Eagle's medium (DMEM), 5% fetal bovine serum, 2 mM L-glutamine, 10 mM HEPES, 1 mM sodium pyruvate, 0.5 mg/ml Zeocin, and 0.5 mg/ml Geneticin). After 24 hrs at 37° C., medium is removed and replaced with 50 μl of dye loading buffer (Hank's balanced salt solution (HBSS) containing 25 mM HEPES, 0.04% Pluronate 127 and 8 μM Fluo3 Both from Molecular Probes)). After a 60 min loading period at room temperature, dye loading buffer is aspirated and replaced with 100 μl of HEPES/IBBS. Plate is placed in FLIPR and basal readings are taken for 10 sec, at which point 100 μl of buffer containing 2 μM MCH (1 μM final) is added and measurements are taken over 105 sec. To correct for variations between clones in numbers of cells per well, the MCH response is normalized to the response induced by epinephrine.

Both the ¹²⁵I-MCH binding and functional GTPγ³⁵S binding assays employed membranes isolated from a clone designated as clone 43. Typically, cells from 20 confluent T225 flasks were processed by washing the monolayers in cold phosphate-buffered saline (PBS), scraping the cells into same and re-suspending the cell pellet in 35 ml of 250 mM Sucrose, 50 mM HEPES, pH 7.5, 1 mM MgCl₂, 24 μg/ml DNase I, and protease inhibitors (1 Complete® tablet, per 50 ml of buffer prepared, Roche Diagnostics). After incubation on ice for 5 min, cells were disrupted with 20-25 strokes of a Teflon/Glass homogenizer attached to an overhead motorized stirrer, and the homogenate was centrifuged at 40,000 rpm in Beckman Type 70.1 Ti rotor. The pellets were re-suspended in 250 mM Sucrose, 50 mM HEPES, pH 7.5, 1.5 mM CaCl₂, 1 mM MgSO₄ and protease inhibitors by Teflon/Glass homogenization to achieve a protein concentration of ˜3-5 mg/ml (Pierce BCA assay with Bovine serum albumin as standard). Aliquots were stored at −70° C.

Binding of compounds to MCHR1 was assessed in a competitive binding assay employing ¹²⁵I-MCH, compound and clone 43 membranes. Briefly, assays are carried out in 96-well Costar 3632 white opaque plates in a total volume of 200 μl containing 25 mM HEPES, pH 7.5, 10 mM CaCl₂, 2 mg/ml bovine serum albumin, 0.5% dimethyl sulfoxide (DMSO), 4 μg of clone 43 membranes, 100 pM ¹²⁵I-MCH (NEN), 1.0 mg of wheat germ agglutinin scintillation proximity assay beads (WGA-SPA beads, Amersham) and a graded dose of test compound. Non-specific binding is assessed in the presence of 1 μM unlabeled MCH. Bound ¹²⁵I-MCH is determined by placing sealed plates in a Microbeta Trilux (Wallac) and counting after a 5 hr delay.

IC₅₀ values (defined as the concentration of test compound required to reduce specific binding of ¹²⁵I-MCH by 50%) are determined by fitting the concentration-response data to a 4-parameter model (max response, min response, Hill coefficient, IC₅₀) using Excel. Kᵢ values are calculated from IC₅₀ values using the Cheng-Prusoff approximation as described by Cheng et al. (Relationship between the inhibition constant (Kᵢ) and the concentration of inhibitor which causes 50% inhibition (IC₅₀) of an enzymatic reaction, Biochem. Pharmacol., 22: 3099-3108 (1973)). The K_{d} for ¹²⁵I-MCH is determined independently from a saturation binding isotherm.

Functional antagonism of MCH activity is assessed by measuring the ability of test compound to inhibit MCH-stimulated binding of GTPγ³⁵S to clone 43 membranes.

Briefly, assays are carried out in Costar 3632 white opaque plates in a total volume of 200 μl containing 25 mM Hepes, pH 7.5, 5 mM MgCl₂, 10 μg/ml saponin, 100 mM NaCl, 3 μM GDP, 0.3 nM GTPγ³⁵S, 40 nM MCH (approximately equal to EC₉₀), 20 μg of clone 43 membranes, 1.0 mg of wheat germ agglutinin scintillation proximity assay beads (WGA-SPA beads, Amersham) and a graded dose of test compound. The plates are sealed and left for 16-18 hrs at 4° C. After a 1 hr delay to allow plates to equilibrate to ambient temperature, bound GTPγ³⁵S is determined by counting in a Microbeta Trilux (Wallac).

IC₅₀ values (defined as the concentration of test compound required to reduce MCH-stimulated GTPγ³⁵S binding by 50%) are determined by fitting the concentration-response data to a 4-parameter model (max response, min response, Hill coefficient, IC₅₀) using Excel. K_{b} values are calculated from IC₅₀ values using a modification of the Cheng-Prusoff approximation as described by Leff and Dougal (Further concerns over Cheng-Prusoff analysis, Trends Pharmacol. Sci. 14: 110-112 (1993)) after verifying competitive antagonism by Schild analysis. The EC₅₀ for MCH alone is determined independently. The MCHR1 binding and functional activities of 24 compounds in the oxadiazole series (tested in duplicate) are shown in Table 1

TABLE 1

| Structure | | Kᵢ (nM) | K_{b} (nM) |
|---|---|---|---|
| 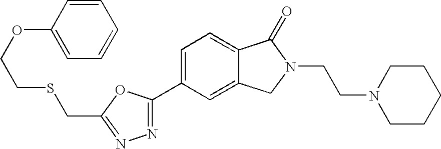 | | 1066 | — |
| 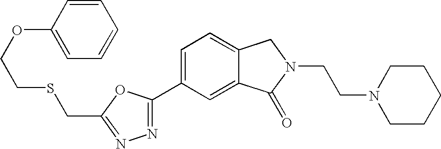 | | 2000 | — |
| 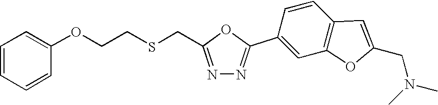 | | 372.1 | — |
| 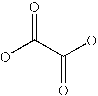 | | | |
| 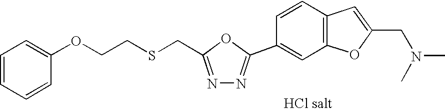 | | 383.3 | — |
| 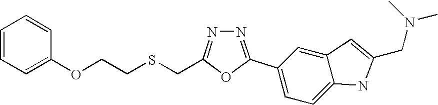 | | 189.7 | — |
| 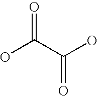 | | | |
| 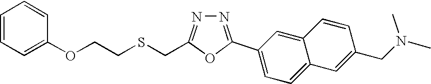 | | 8.2 | 11.8 |
| 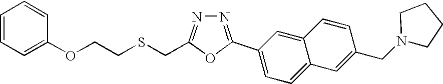 | | 7.3 | 7.0 |
| 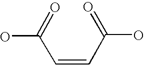 | | | |
| 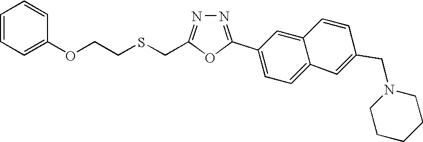 | | 17.6 | 58.4 |
| | 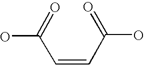 | 682.4 | — |

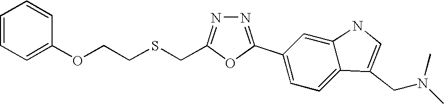

| | | |
|---|---|---|
| 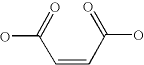 | 234.6 | — |

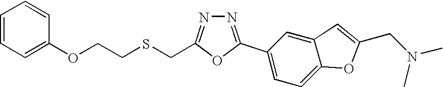

| | 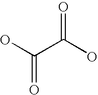 | 153.1 | — |
|---|---|---|---|
| 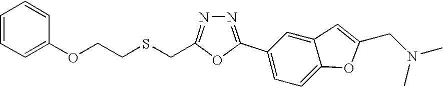 | | | |
| 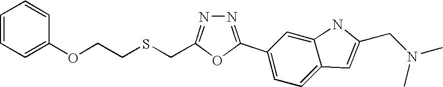 | | 34.6 | — |
| | 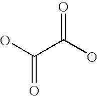 | 11085.9 | — |

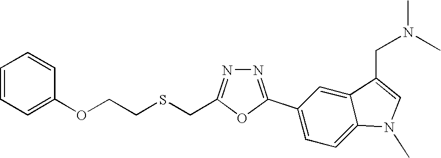

### Utility

As antagonists of the MCHR1 binding, a compound of the present invention is useful in treating conditions in human and non-human animals in which the the MCHR1 receptor has been demonstrated to play a role. The diseases, disorders or conditions for which compounds of the present invention are useful in treating or preventing include, but are not limited to, diabetes mellitus, hyperglycemia, obesity, hyperlipidemia, hypertriglyceridemaia, hypercholesterolemia, atherosclerosis of coronary, cerebrovascular and peripheral arteries, gastrointestinal disorders including peptid ulcer, esophagitis, gastritis and duodenitis, (including that induced by H. pylori), intestinal ulcerations (including inflammatory bowel disease, ulcerative colitis, Crohn's disease and proctitis) and gastrointestinal ulcerations, neurogenic inflammation of airways, including cough, asthma, depression, prostate diseases such as benign prostate hyperplasia, irritable bowel syndrome and other disorders needing decreased gut motility, diabetic retinopathy, neuropathic bladder dysfunction, elevated intraocular pressure and glaucoma and non-specific diarrhea dumping syndrome. By inhibiting the MCH activity the compounds of the invention provide anorexic effects as weight loss agents singly or in combination with other effective weight loss agents and/or exercise. That is, the compounds of the invention are useful as appetite suppressants and/or weight loss agents. Compounds of the present invention have also shown some affinity for the R₂ isoform of MCHR. The compounds of the invention may also be used in combination with other approved therapeutic agents for the treatment and/or prevention of obesity and related diseases. In this format, the compounds of the present invention exhibit the positive effects of such approved combination treatments while minimizing the side effects due to the potential requirement of lower doses of such combination compounds. Such combination therapies may be delivered individually or in a combined formulation. Examples of compounds potentially useful in combination with compounds of formula I include weight loss agents (Mevidia™, Xenical™), cholesterol lowering agents, glucose level control or modulating agents and the like.

In treating non-human, non-companion animals, the compounds of the present invention are useful for reducing weight gain and/or improving the feed utilization efficiency and/or increasing lean body mass.

### Formulation

The compound of formula I is preferably formulated in a unit dosage form prior to administration. Therefore, yet another embodiment of the present invention is a pharmaceutical formulation comprising a compound of formula I and a pharmaceutical carrier.

The present pharmaceutical formulations are prepared by known procedures using well-known and readily available ingredients. In making the formulations of the present invention, the active ingredient (formula I compound) will usually be mixed with a carrier, or diluted by a carrier, or enclosed within a carrier which may be in the form of a liquid, tablet, capsule, sachet, paper or other container. When the carrier serves as a diluent, it may be a solid, semisolid or liquid material which acts as a vehicle, excipient or medium for the active ingredient. Thus, the compositions can be in the form of tablets, pills, powders, lozenges, sachets, cachets, elixirs, suspensions, emulsions, solutions, syrups, aerosol (as a solid or in a liquid medium), soft and hard gelatin capsules, suppositories, sterile injectable solutions and sterile packaged powders.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient.

### FORMULATION EXAMPLES

Formulation 1
Tablets

| Ingredient | Quantity (mg/tablet) |
|---|---|
| Active Ingredient | 5-500 |
| Cellulose, microcrystalline | 200-650 |
| Silicon dioxide, fumed | 10-650 |
| Stearate acid | 5-15 |

The components are blended and compressed to form tablets.

Formulation 2
Suspensions

| Ingredient | Quantity (mg/5 ml) | |
|---|---|---|
| Active Ingredient | 5-500 | mg |
| Sodium carboxymethyl cellulose | 50 | mg |
| Syrup | 1.25 | mg |
| Benzoic acid solution | 0.10 | ml |
| Flavor | q.v. | |
| Color | q.v. | |
| Purified water to | 5 | ml |

The medicament is passed through a No. 45 mesh U.S. sieve (approximately 355 micron opening) and mixed with the sodium carboxymethyl cellulose and syrup to form a smooth paste. The benzoic acid solution, flavor, and color are diluted with some of the water and added, with stirring. Sufficient water is then added to produce the required volume.

Formulation 3
Intravenous Solution

| Ingredient | Quantity | |
|---|---|---|
| Active Ingredient | 25 | mg |
| Isotonic saline | 1,000 | ml |

The solution of the above ingredients is intravenously administered to a patient at a rate of about 1 ml per minute.

Dose

The specific dose administered is determined by the particular circumstances surrounding each situation. These circumstances include, the route of administration, the prior medical history of the recipient, the pathological condition or symptom being treated, the severity of the condition/symptom being treated, and the age and sex of the recipient. However, it will be understood that the therapeutic dosage administered will be determined by the physician in the light of the relevant circumstances, or by the vetrinarian for non-human recipients.

Generally, an effective minimum daily dose of a compound of formula I is about 5, 10, 15, or 20 mg. Typically, an effective maximum dose is about 500, 100, 60, 50, or 40 mg. Most typically, the dose ranges between 5 mg and 60 mg. The exact dose may be determined, in accordance with the standard practice in the medical arts of “dose titrating” the recipient; that is, initially administering a low dose of the compound, and gradually increasing the does until the desired therapeutic effect is observed.

Route of Administration

The compounds may be administered by a variety of routes including the oral, rectal, transdermal, subcutaneous, topical, intravenous, intramuscular or intranasal routes.

Combination Therapy

A compound of formula I may be used in combination with other drugs or therapies that are used in the treatment/prevention/suppression or amelioration of the diseases or conditions for which compounds of formula I are useful. Such other drug(s) may be administered, by a route and in an amount commonly used therefor, contemporaneously or sequentially with a compound of formula I. When a compound of formula I is used contemporaneously with one or more other drugs, a pharmaceutical unit dosage form containing such other drugs in addition to the compound of formula I is preferred. Accordingly, the pharmaceutical compositions of the present invention include those that also contain one or more other active ingredients, in addition to a compound of formula I. Examples of other active ingredients that may be combined with a compound of formula I, either administered separately or in the same pharmaceutical compositions, include, but are not limited to:
(a) insulin sensitizers including (i) PPARγ agonists such as the glitazones (e.g. troglitazone, pioglitazone, englitazone, MCC-555, BRL49653 and the like), and compounds disclosed in W097/27857, 97/28115, 97/28137 and 97/27847; (ii) biguanides such as metformin and phenformin;
(b) insulin or insulin mimetics;
(c) sulfonylureas such as tolbutamide and glipizide;
(d) alpha-glucosidase inhibitors (such as acarbose);
(e) cholesterol lowering agents such as
   i. HMG-CoA reductase inhibitors (lovastatin, simvastatin and pravastatin, fluvastatin, atorvastatin, and other statins),
   ii. sequestrants (cholestyramine, colestipol and a dialkylaminoalkyl derivatives of a cross-linked dextran),
   iii. nicotinyl alcohol nicotinic acid or a salt thereof,
   iv. proliferator-activator receptor a agonists such as fenofibric acid derivatives (gemfibrozil, clofibrat, fenofibrate and benzafibrate),
   v. inhibitors of cholesterol absorption for example β-sitosterol and (acyl CoA:cholesterol acyltransferase) inhibitors for example melinamide,
   vi. probucol,
   vii. vitamin E, and
   viii. thyromimetics;
(f) PPARδ agonists such as those disclosed in WO97/28149;
(g) antiobesity compounds such as fenfluramine, dexfenfluramine, phentermine, sibutramine, orlistat, and other β₃ adrenergic receptor agonists;
(h) feeding behavior modifying agents such as neuropeptide Y antagonists (e.g. neuropeptide Y5) such as those disclosed in WO 97/19682, W0 97/20820, WO 97/20821, WO 97/20822 and WO 97/20823;
(i) PPARα agonists such as described in WO 97/36579 by Glaxo;
(j) PPARγ antagonists as described in WO 97110813; and
(k) serotonin reuptake inhibitors such as fluoxetine and sertraline
(l) antipsychotic agents such as for example olanzapine.

### Experimental Section

### Preparation of 2-dimethylaminomethyl-benzofuran-6-carboxylic acid N′-[2-(2-phenoxy-ethylsulfanyl)-acetyl]-hydrazide.

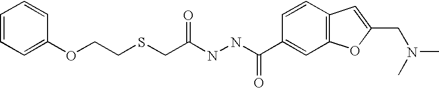

### a) 3-Hydroxy-4-iodo-benzoic acid

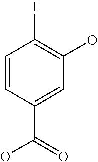

The compound 3-hydroxy-4-iodo-benzoic acid was synthesized as described in J. C. S. Perkin I, 1995, 1103-1113 to obtain a white solid after workup.

### b) 3-Hydroxy-4-iodobenzoic acid methyl ester

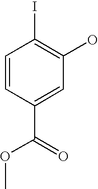

The compound 3-Hydroxy-4-iodobenzoic acid methyl ester was synthesized as described in J. C. S. Perkin I 1995, 1103-1113 to obtain a tan solid after workup.

### c) 2-Dimethylaminomethyl-benzofuran-6-carboxylic acid methyl ester oxalate

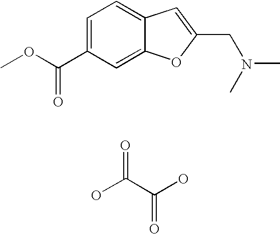

An anhydrous acetonitrile solution of 3-hydroxy-4-iodo-benzoic acid methyl ester (1.5 g, 5.39 mmol, 1 eq.) was treated with copper (I) iodide (0.26 g, 1.35 mmol, 0.25 eq.), dichlorobis(triphenylphosphine)palladium(II) (0.38 g, 0.54 mmol, 0.1 eq.), triethylamine (5 mL), and 1-dimethylamino-2-propyne (1.34 g, 16.17 mmol, 1.74 mL, 3 eq.). The reaction was allowed to stir at 70° C. overnight. The solvent was removed in vacuo and the resulting oil was purified via silica gel chromatography using 2.5% MeOH in CHCl₃ as the mobile phase to obtain 2-dimethylaminomethyl-benzofuran-6-carboxylic acid as an orange/brown oil.

The oil was converted to the oxalate salt by adding 1.2 eq. of oxalic acid (0.58 g) in acetone dropwise to an acetone solution of the amine to obtain 2-dimethylamninomethyl-benzofuran-6-carboxylic acid methyl ester oxalate (1.6372 g, 94% yield) as a tan solid.

¹H NMR (d6-DMSO) δ 8.12 (s, 1H), 7.89 (m, 1H), 7.80 (m, 1H), 7.14 (s, 1H) 4.23 (s, 2H), 3.89 (s, 3H), 2.60 (s, 6H). IR (KBr, cm⁻¹) 3385.5, 3118.4, 2990.1, 2949.6, 2694.1, 2605.4, 1704.8, 1625.7, 1433.8, 1285.3, 1225.6, 1194.7, 989.3, 764.6, 702.9. MS (ES⁺) m/z 234 [M+H]⁺, 189 [M−N(CH₃)₂]⁺. Analytical composition calculated for C₁₅H₁₇NO₇ C, 55.73; H, 5.30; N, 4.33. Found C, 55.95; H, 5.31; N, 4.33. M.P. 185-187° C.

### d) 2-Dimethylaminomethyl-benzofuran-6-carboxylic acid hydrazide

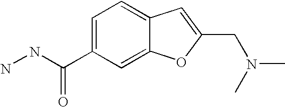

A methanol solution of 2-dimethylaminomethyl-benzofuran-6-carboxylic acid methyl ester (4.2 g, 17.98 mmol, 1 eq.) was treated with hydrazine (2.88 g, 89.9 mmol, 5 eq.) and the solution heated to reflux. When the reaction was complete, the solvent was removed in vacuo and the residue dissolved in ethyl acetate and washed 2×150 mL with water and then brine. The organic layer was collected, dried over MgSO₄, filtered, and the solvent removed leaving an orange oil that was purified via normal phase chromatography leaving 2-dimethylaminomethyl-benzofuran-6-carboxylic acid hydrazide (2.13 g, 51% yield) as an orange oil after removal of the solvent in vacuo.

¹H NMR (d6-DMSO) δ 9.77 (s, 1H), 7.98 (s, 1H), 7.73 (m, 1H), 7.62 (m, 1H), 6.82 (s, 1H), 4.49 (s, 2H), 3.61 (s, 2H), 2.22 (s, 6H). MS (ES⁺) m/z 234 [M+H]⁺, 189 [M−N(CH₃)₂]⁺.

### e) 2-Dimethylaminomethyl-benzofuran-6-carboxylic acid N′-[2-(2-phenoxy-ethylsulfanyl)-acetyl]-hydrazide

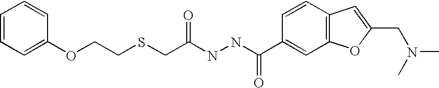

A 20% THF in AcCN solution of (2-phenoxy-ethylsulfanyl)-acetic acid (1.87 g, 8.79 mmol, 1 eq.) was treated with EEDQ (2.39 g, 9.67 mmol, 1.1 eq.) and the solution allowed to stir for 1 hour at room temperature. This solution was then treated with 2-dimethylaminomethyl-benzofuran-6-carboxylic acid hydrazide (2.05 g, 8.79 mmol, 1 eq.) as a solid and the resulting solution allowed to stir at room temperature overnight.

Removed the solvent in vacuo leaving an orange oil which was purified via normal phase chromatography leaving 2-dimethylaminomethyl-benzofuran-6-carboxylic acid N′-[2-(2-phenoxy-ethylsulfanyl)-acetyl]-hydrazide (2.03 g, 54% yield) as an orange oil.

¹H NMR (d6-DMSO) δ 10.52 (s, 1H), 10.16 (s, 1H), 8.10 (s, 1H), 7.80 (m, 2H), 7.29 (m, 2H), 7.11 (s, 1H), 6.95 (m, 3H), 4.21 (m, 4H), 3.35 (s, 2H), 3.06 (t, 2H, J=7 Hz), 2.60 (s, 6H). IR (KBr, cm⁻¹) 3426.9, 3262.1, 3037.4, 1696.1, 1659.5, 1496.5, 1290.2, 1242.9, 947.9,756.9, 693.3. MS (ES⁺) m/z 428 [M+H]⁺. MS (ES⁻) m/z 426 [M−H]⁻. M.P. 162-164° C.

### Example 1

### Preparation of dimethyl-{6-[5-(2-phenoxy-ethylsulfanylmethyl)-[1,3,4]oxadiazol-2-yl]-benzofuran-2-ylmethyl }-amine oxalate

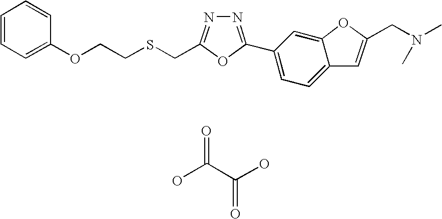

A THF solution of 2-dimethylaminomethyl-benzofuran-6-carboxylic acid N′-[2-(2-phenoxy-ethylsulfanyl)-acetyl]-hydrazide (2.2 g, 5.15 mmol, 1 eq.) was treated with triethylamine (1.88 g, 18.54 mmol, 2.58 mL, 3.6 eq.), triphenylphosphine (1.49 g, 5.67 mmol, 1.1 eq.), and carbon-tetrabromide (2.05 g, 6.18 mmol, 1.2 eq.). The solution was allowed to stir at room temperature overnight.

The solvent was removed from the suspension leaving a brown oil that was purified via normal phase chromatography leaving dimethyl-{6-[5-(2-phenoxy-ethylsulfanylmethyl)-[1,3,4]oxadiazol-2-yl]-benzofuran-2-ylmethyl}-amine as an orange oil contaminated with triphenylphosphine oxide. The oil was converted to the oxalate salt by adding an acetone solution of oxalic acid to an acetone solution of the amine. Obtained dimethyl-{6-[5-(2-phenoxy-ethylsulfanylmethyl)-[1,3,4]oxadiazol-2-yl]-benzofuran-2-ylmethyl}-amine oxalate (0.5244 g, 43% yield) as an off-white solid by filtration.

¹H NMR (d6-DMSO) δ 8.14 (s, 1H), 7.89 (s, 2H), 7.26 (m, 2H), 7.16 (s, 1H), 6.92 (m, 3H), 4.21 (m, 6H), 3.05 (t, 2H, J=7 Hz), 2.60 (s, 6H). IR (KBr, cm⁻¹) 3395.1, 3022.9 2981.5, 2545.6, 1719.3, 1600.6, 1553.4, 1459.9, 1190.8, 702.9. MS (ES⁺) m/z410 [M+H]⁺. Analytical composition calculated for C₂₄H₂₅N₃O₇S C, 57.70; H, 5.04; N, 8.41. Found C, 57.42; H, 4.96; N, 8.40. Analytical HPLC 100% purity. M.P. softening at 150° C. and then 161-162° C.

### Preparation of 2-dimethylaminomethyl-benzofuran-5-carboxylic acid N′-[2-(2-phenoxy-ethylsulfanyl)-acetyl]-hydrazide

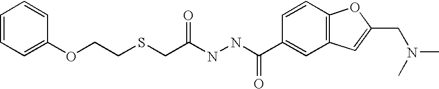

### a) 4-Hydroxy-3-iodo-benzoic acid methyl ester

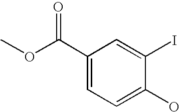

Methyl 4-hydroxybenzoate (15.22 g, 100 mmol, 1 eq.), sodium iodide (14.99 g, 100 mmol, 1 eq.), and sodium hydroxide (4.0 g, 100 mmol, 1 eq.) were dissolved in 250 mL of cold methanol and treated with sodium hypochlorite (5.25% aqueous solution, 142 mL, 100 mmol, 1 eq.) keeping the temperature below 3° C. When addition of the sodium hypochlorite was complete, the reaction was allowed to stir at 0° C. After 1 hour, 100 mL of 10% sodium thiosulfate was added followed by pH adjustment of the reaction with 5 M aqueous HCl until the pH was about 2. The resulting white solid was collected by filtration and washed with water leaving 4-hydroxy-3-iodo-benzoic acid methyl ester (19.52 g, 70% yield).

¹H NMR (d6-DMSO) δ 11.30 (s, 1H), 8.22 (m, 1H), 7.81 (m, 1H), 6.96 (d, 1H, J=8.4 Hz), 3.79 (s, 3H). MS (TOF MS EI⁺) m/z 278 [M⁺]. IR (CHCl₃; cm⁻¹) 3579.3, 3486.7, 3209.0, 3032.6, 2954.5, 1716.4, 1594.9, 1436.7, 1283.4, 1183.1, 1115.6, 972.0. M.P. 142-150° C.

### b) 2-Dimethylaminomethyl-benzofuran-5-carboxylic acid methyl ester

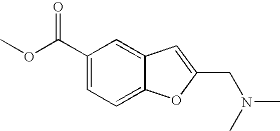

A DMF solution of 4-hydroxy-3-iodo-benzoic acid methyl ester (7.70 g, 27.69 mmol, 1 eq.) was treated with 1-dimethylamino-2-propyne (3.45 g, 4.47 mL, 41.54 mmol, 1.5 eq.), copper (I) iodide (0.42 g, 2.22 mmol, 0.08 eq.), dichlorobis(triphenylphosphine)palladium(II) (0.97 g, 1.38 mmol, 0.05 eq.), and triethylamine (11 mL) and heated to 75° C. for 2 hours. The reaction was diluted with diethyl ether and washed with water and then 50% brine. The organic layer was collected, dried, filtered, and the solvent removed leaving a dark brown oil which was purified by normal phase chromatography using a step gradient of 2M NH₃ in methanol in dichloromethane as the mobile phase to obtain 2-dimethylaminomethyl-benzofuran-5-carboxylic acid methyl ester (5.58 g, 86% yield) as a brown oil.

¹H NMR (d6-DMSO) δ 8.35 (m, 1H), 7.96 (m, 1H), 7.71 (d, 1H, J=8.8 Hz), 7.15 (s, 1H), 4.20 (s, 2H), 3.88 (s, 3H), 2.58 (s, 6H). MS (ES⁺) m/z 234 [M+H]⁺, 189 [M−N(CH₃)₂]⁺. IR (KBr, cm⁻¹) 3390.3, 3010.2, 2954.7, 2642.8, 2604.2, 2477.7, 1713.7, 1618.9, 1427.0, 1303.8, 1242.8, 1191.3, 1143.2, 1086.4, 954.0, 840.0, 764.9, 720.7. Analytical LC/MS indicates 100% purity (diode array detector). M.P. gradual darkening and melting at 182-184° C.

### c) 2-Dimethylaminomethyl-benzofuran-5-carboxylic acid hydrazide

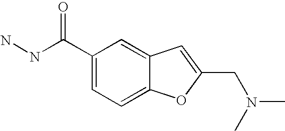

An ethanolic solution of 2-dimethylaminomethyl-benzofuran-5-carboxylic acid methyl ester (5.0 g, 21.43 mmol, 1 eq.) was treated with hydrazine (3.43 g, 107.15 mmol, 5 eq.) and the solution heated to reflux. When the reaction was complete, the solvent was removed in vacuo and the yellow residue purified via normal phase chromatography leaving 2-dimethylaminomethyl-benzofuran-5-carboxylic acid hydrazide (4.43 g, 89% yield) as an orange oil

¹H NMR (d6-DMSO) δ 9.74 (s, 1H), 8.08 (m, 1H), 7.76 (m, 1H), 7.57 (d, 1H, J=8.4 Hz), 6.84 (s, 1H), 4.44 (s, 2H), 3.60 (s, 2H), 2.22 (s, 6H). MS(ES⁺) m/z 234 [M+H]⁺, 189 [M−N(CH₃)₂]⁺. IR (CHCl₃, cm⁻¹) 3668.0, 3449.1, 3329.8, 3009.6, 2950.9, 2827.3, 2781.0, 1666.0, 1627.2, 1495.9, 1458.5, 1317.1, 1268.9, 1022.3, 845.8. M.P. softening at 75° C. and then melting at 82-92° C.

### d) 2-Dimethylaminomethyl-benzofuran-5-carboxylic acid N′-[2-(2-phenoxy-ethylsulfanyl)-acetyl]-hydrazide

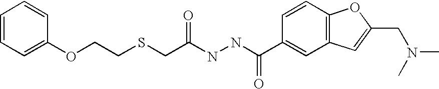

A 20% THF in AcCN solution of (2-phenoxy-ethylsulfanyl)-acetic acid (3.64 g, 17.15 mmol, 1 eq.) was treated with EEDQ (4.66 g, 18.86 mmol, 1.1 eq.) and the solution allowed to stir for 1 hour at room temperature. This solution was then treated with 2-dimethylaminomethyl-benzofuran-5-carboxylic acid hydrazide (4.0 g, 17.15 mmol, 1 eq.) and the resulting solution allowed to stir at room temperature overnight.

Removed the solvent in vacuo leaving a brown oil which was purified via normal phase chromatography leaving 2-dimethylaminomethyl-benzofuran-5-carboxylic acid N′-[2-(2-phenoxy-ethylsulfanyl)-acetyl]-hydrazide (4.98 g, 68% yield) as an orange oil. A small portion of the compound was converted to the maleate salt.

¹H NMR (d6-DMSO) δ 8.37 (m, 1H), 8.00 (m, 1H), 7.84 (d, 1H, J=8.8 Hz), 7.26 (m, 3H), 6.92 (m, 3H), 6.08 (s, 2H), 4.54 (s, 2H), 4.25 (s, 2H), 4.20 (t, 2H, j=6.6 Hz), 3.05 (t, 2H, J=6.6 Hz), 2.80 (s, 6H). MS(ES⁺) m/z 428 [M+H]+. IR(KBr, cm⁻¹) 3431.8, 3284.2, 3206.2, 3020.2, 1704.7, 1661.4, 1582.2, 1497.1, 1364.4, 1297.6, 1237.1, 1180.2, 1114.7, 1014.5, 874.8, 753.2. Analytical composition calculated for C₂₆H₂₉N₃O₈S C, 57.45; H, 5.40; N, 7.73. Found C, 57.27; H, 5.41; N, 7.73. Pound C, 57.27; H, 5.41, N, 7.74. Analytical LC/MS: 100% purity (diode array detector). M.P. 127-129° C.

### Example 2

### Preparation of dimethyl-{5-[5-(2-phenoxy-ethylsulfanylmethyl)-[1,3,4]oxadiazol-2-yl]-benzofuran-2-ylmethyl}-amine oxalate

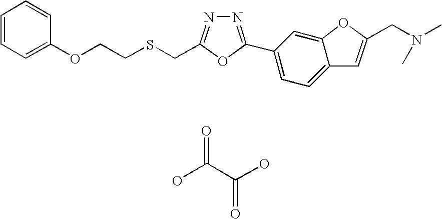

A THF solution of 2-dimethylaminomethyl-benzofuran-5-carboxylic acid N′-[2-(2-phenoxy-ethylsulfanyl)-acetyl]-hydrazide (4.0 g, 9.36 mmol, 1 eq.) was treated with triethylamine (3.41 g, 33.7 mmol, 4.7 mL, 3.6 eq.), triphenylphosphine (2.70 g, 10.3 mmol, 1.1 eq.), and carbon tetrabromide (3.72 g, 11.23 mmol, 1.2 eq.). The solution was allowed to stir at room temperature overnight.

The solvent was removed from the suspension leaving a brown oil that was purified via normal phase chromatography leaving dimethyl-{5-[5-(2-phenoxy-ethylsulfanylmethyl)-[1,3,4]oxadiazol-2-yl]-benzofuran-2-ylmethyl{-amine as an orange oil contaminated with triphenylphosphine oxide. The oil was converted to the oxalate salt by adding an acetone solution of oxalic acid to an acetone solution of the amine. Obtained dimethyl-{5-[5-(2-phenoxy-ethylsulfanylmethyl)-[1,3,4]oxadiazol-2-yl]-benzofuran-2-ylmethyl}-amine oxalate (2.1557 g, 46% yield) as an off-white solid by filtration.

¹H NMR (d6-DMSO) δ 8.37 (m, 1H), 8.00 (m, 1H), 7.84 (d, 1H, J=8.8 Hz), 7.26 (m, 3H), 6.92 (m, 3H), 6.07 (s, 2H), 4.53 (s, 2H), 4.25 (s, 2H), 4.21 (t, 2H, J=6.6 Hz), 3.05 (t, 2H, J=6.6 Hz), 2.79 (s, 6H). MS(ES⁺) m/z 410 [M+H]⁺. IR (KBr, cm⁻¹) 3432.8, 2989.4, 2940.2, 1704.4, 1582.6, 1462.3, 1383.0, 1354.3, 1244.9, 1171.7, 1074.6, 1016.0, 969.5, 866.7, 819.6, 758.4. Analytical LC/MS: 100% purity (diode array detector). M.P. 138-140° C.

### Preparation of {1-methanesulfonyl-5-[5-(2-phenoxy-ethylsulfanylmethyl)-[1,3,4]oxadiazol-2-yl]-1H-indol-2-ylmethyl }-dimethyl-amine

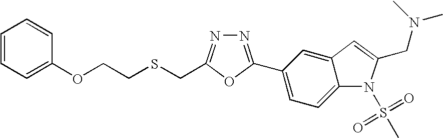

### a) 3-Iodo-4-methanesulfonylamino-benzoic acid methyl ester

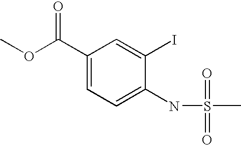

A pyridine solution of methyl 4-amino-3-iodobenzoate (10 g, 36.09 mmol, 1 eq.) was treated dropwise with methanesulfonyl chloride (6.20 g, 4.2 mL, 54.14 mmol, 1.5 eq.) and the reaction stirred overnight. The pyridine was removed in vacuo and the residue diluted with ethyl acetate and washed with water. The organic layers were combined, dried over MgSO₄, filtered, and the solvent removed in vacuo leaving an orange solid that was recrystallized from CH₂Cl₂/hexane. 3-Iodo-4-methanesulfonylamino-benzoic acid methyl ester (12.79 g, 99% yield) was obtained by collection of the orange solid by filtration.

¹H NMR (d6-DMSO) δ 8.47 (m, 1H), 8.01 (m, 1H), 7.79 (d, 1H, J=8.4), 3.89 (s, 3H), 3.66 (s, 3H). MS(ES⁻) m/z 354 [M−H]⁻.

### b) 2-Dimethylaminomethyl-1-methanesulfonyl-1H-indole-5-carboxylic acid methyl ester oxalate

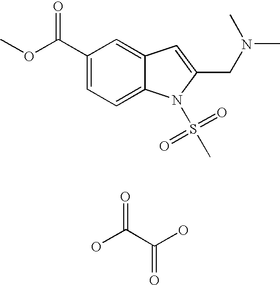

A DMF solution of 3-iodo-4-methanesulfonylamino-benzoic acid methyl ester (6.8 g, 19.15 mmol, 1 eq.) was treated with 1-dimethylamino-2-propyne (2.39 g, 28.73 mmol, 1.5 mmol), copper(I) iodide (0.29 g, 1.53 mmol, 0.08 mmol), dichlorobis(triphenylphosphine)palladium(II) (0.67 g, 0.96 mmol, 0.05 mmol), and triethylamine (8 mL) and the dark reaction allowed to stir at 80° C. overnight. Filtered the reaction through celite to remove Pd and the filtrate diluted with diethyl ether. The organic layer was washed with water and then 50% brine. The organic layer was collected, dried over MgSO₄, filtered, and the solvent removed in vacuo leaving an orange oil.

The oil was purified via normal phase chromatography using a step gradient of ethyl acetate in hexanes leaving 2-dimethylaminomethyl-1-methanesulfonyl-1H-indole-5carboxylic acid methyl ester (4.55 g, 77% yield) as an orange/brown oil that solidified on standing.

A small portion of 2-dimethylaminomethyl-1-methanesulfonyl-1H-indole-5-carboxylic acid methyl ester was converted to the oxalate salt and 2-dimethylaminomethyl-1-methanesulfonyl-1H-indole-5-carboxylic acid methyl ester oxalate was collected as a white solid.

¹H NMR (d6-DMSO) δ 8.33 (s, 1H), 8.01 (m, 2H), 7.08 (s, 1H), 4.15 (s, 2H), 3.89 (s, 3H), 3.62 (s, 3H), 2.51 (s, 6H). MS(ES⁺) m/z 311 [M+H]⁺, 266 [M−N(CH₃)₂]⁺. IR (KBr, cm⁻¹) 3408.6, 3027.7, 3013.3, 1711.1, 1612.0, 1433.6, 1367.7, 1306.6, 1267.0, 1215.0, 1168.3, 1127.9, 975.2, 774.9. Analytical composition calculated for C₁₆H₂₀N₂O₈S (oxalate salt) C, 48.00; H, 5.03; N, 7.00. Found C, 47.63; H, 5.03; N, 6.91. Analytical LC/MS: 100% (diode array detector). M.P. 190-193° C.

### c) 2-Dimethylaminomethyl-1-methanesulfonyl-1H-indole-5-carboxylic acid hydrazide

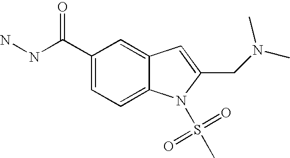

2-Dimethylaminomethyl-1-methanesulfonyl-1H-indole-5-carboxylic acid methyl ester (5.93 g, 19.11 mmol, 1 eq.) was converted to 2-dimethylaminomethyl-1-methanesulfonyl-1H-indole-5-carboxylic acid hydrazide in the same way as described for 2-dimethylaminomethyl-benzofuran-6-carboxylic acid hydrazide. Obtained 2-dimethylaminomethyl-1-methanesulfonyl-1H-indole-5-carboxylic acid hydrazide (4.77 g, 80% yield) as a yellow solid after workup and normal phase chromatography.

¹H NMR (d6-DMSO) δ 9.74 (s, 1H), 8.08 (m, 1H), 7.94 (m, 1H), 7.78 (m, 1H), 6.80 (s, 1H), 4.47 (s, 2H), 3.68 (s, 2H), 3.62 (3H), 2.21 (s, 6H). MS(ES⁺) m/z 311 [M+H]⁺, 266 [M−N(CH₃)₂]⁺. IR (KBr, cm⁻¹) 3319.0, 3278.4, 3096.2, 3029.7, 2950.6, 2820.4, 2773.2, 1653.7, 1609.8, 1530.8, 1346.3, 1224.3, 1166.1, 1140.1, 1054.1, 978.1, 967.1, 775.0.

### d) 2-Dimethylaminomethyl-1-methanesulfonyl-1H-indole-5-carboxylic acid N′-[2-(2-phenoxy-ethylsulfanyl)-acetyl]-hydrazide

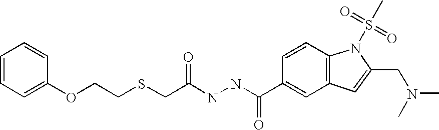

(2-Phenoxy-ethylsulfanyl)-acetic acid (2.59 g, 12.21 mmol, 1 eq.) was treated with EEDQ (3.32 g, 13.43 mmol, 1.1 eq.) and 2-dimethylaminomethyl-1-methanesulfonyl-1H-indole-5-carboxylic acid hydrazide (3.79 g, 12.21 mmol, 1 eq.) as described for 2-dimethylaminomethyl-benzofuran-6-carboxylic acid N′-[2-(2-phenoxy-ethylsulfanyl)-acetyl]-hydrazide to obtain 2-dimethylaminomethyl-1-methanesulfonyl-1H-indole-5-carboxylic acid N′-[2-(2-phenoxy-ethylsulfanyl)-acetyl]-hydrazide (5.57 g, 90% yield) as an orange oil.

¹H NMR (d6-DMSO) δ 10.50 (s, 1H), 10.16 (s, 1H), 8.25 (s, 1H), 7.97 (m, 2H), 7.30 (m, 2H), 7.10 (s, 1H), 6.96 (m, 3H), 4.21 (m, 4H), 3.60 (s, 3H), 3.35 (s, 2H), 3.06 (t, 2H, J=6.6 Hz), 2.56 (s, 6H). MS(ES⁺) m/z 505 [M+H]⁺.

### e) {1-Methanesulfonyl-5-[5-(2-phenoxy-ethylsulfanylmethyl)-[1,3,4]oxadiazol-2-yl]-1H-indol-2-ylmethyl }-dimethyl-amine

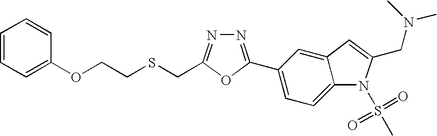

2-Dimethylaminomethyl-1-methanesulfonyl-1H-indole-5-carboxylic acid N′-[2-(2-phenoxy-ethylsulfanyl)-acetyl]-hydrazide (5.17 g, 10.25 mmol, 1 eq.) was converted to {1-methanesulfonyl-5-[5-(2-phenoxy-ethylsulfanylmethyl)-[1,3,4]oxadiazol-2-yl]-1H-indol-2-ylmethyl}-dimethyl-amine as described for dimethyl-{6-[5-(2-phenoxy-ethylsulfanylmethyl)-[1,3,4]oxadiazol-2-yl]-benzofuran-2-ylmethyl }-amine.

¹H NMR (d6-DMSO) δ 8.33 (s, 1H), 8.11 (m, 1H), 8.00 (m, 1H), 7.26 (m, 2H), 7.17 (s, 1H), 6.92 (m, 3H), 4.32 (s, 2H), 4.25 (s, 2H), 4.20 (t, 2H, J=6.2 Hz), 3.63 (s, 3H), 3.04 (t, 2H, J=6.2 Hz), 2.63 (s, 6H). MS(ES⁺) m/z 487 [M+H]⁺. Analytical LC/MS: 100% purity (diode array detector).

### Example 3

### Preparation of dimethyl-{5-[5-(2-phenoxy-ethylsulfanylmethyl)-[1,3,4]oxadiazol-2-yl]-1H-indol-2-ylmethyl}-amine oxalate

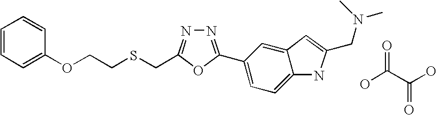

A THF solution of {1-methanesulfonyl-5-[5-(2-phenoxy-ethylsulfanylmethyl)-[1,3,4]oxadiazol-2-yl]-1H-indol-2-ylmethyl}-dimethyl-amine (3.68 g, 7.56 mmol, 1 eq.) was treated with tetrabutylammonium fluoride (1 M in THF, 8.3 mL, 8.3 mmol, 1.1 eq.) and the resulting solution heated to reflux. After 3 hours, removed the THF in vacuo and diluted residue with ethyl acetate and washed with water and brine. Collected the organic layer, dried over MgSO₄, filtered, and dried in vacuo leaving an orange oil in the flask which was purified via normal phase chromatography leaving an orange oil. The free amine was converted to the oxalate salt to obtain dimethyl-{5-[5-(2-phenoxy-ethylsulfanylmethyl)-[1,3,4]oxadiazol-2-yl]-1H-indol-2-ylmethyl }-amine oxalate (0.71 g, 19% yield) as an off-white solid.

¹H NMR (d6-DMSO) δ 11.94 (s, 1H), 8.22 (s, 1H), 7.76 (m, 1H), 7.59 (m, 1H), 7.27 (m, 2H), 6.93 (m, 3H), 6.75 (s, 1H), 4.29 (s, 2H), 4.21 (m, 4H), 3.04 (t, 2H, J−6.2 Hz), 2.67 (s, 6H). MS(ES⁺) m/z 409 [M+H]⁺; 364 [M−N(CH₃)₂]⁺. MS(ES−) m/z 407 [M-H]⁻. IR (KBr, cm⁻¹ ) 3395.1, 3256.3, 1724.1, 1617.4, 1586.4, 1599.6, 1561.1, 1535.4, 1490.8, 1456.7, 1239.6, 1225.9, 711.1. Analytical LC/MS: 100% purity (diode array detector). M.P. 147-150° C.

### Preparation of {1-Methanesulfonyl-6-[5-(2-phenoxyethylsulfanylmethyl)-[1,3,4]oxadiazol-2-yl]-1H-indol-2-ylmethyl}-dimethyl-amine

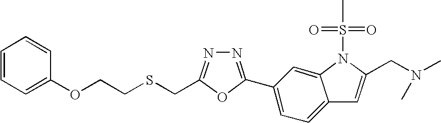

### a) 4-Iodo-3-nitro-benzoic acid

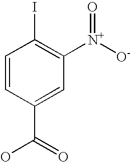

An acetone solution of 4-amino-3-nitrobenzoic acid (25 g, 137.26 mmol, 1 eq.) was cooled in an ice bath and treated with an aqueous solution of sodium nitrite (10.42 g, 150.99 mmol, 1.1 eq.) and the reaction stirred for 30 minutes. The reaction was then treated with an aqueous solution of potassium iodide (23.01 g, 138.63 mmol, 1.01 eq.) and the reaction warmed to 40° C. After 2 hours, the acetone was removed in vacuo and the reaction extracted 2×250 mL with ethyl acetate. The organics were washed with 5M aqueous HCl and then collected, dried, filtered, and the solvent removed in vacuo. The residue was purified by normal phase chromatography using a step gradient of ethyl acetate in hexanes as the mobile phase to give 4-iodo-3-nitro-benzoic acid as an orange solid. This material was taken on to the esterification reaction as is.

### 4Iodo-3-nitro-benzoic acid methyl ester

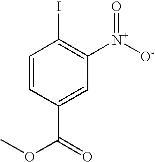

A methanolic solution of 4-iodo-3-nitro-benzoic acid (10.0 g, 34.13 mmol, 1 eq.) was treated with concentrated sulfuric acid (7 mL) and the reaction heated to reflux. After 6 hours, the acid was neutralized with solid sodium bicarbonate and the methanol removed in vacuo. The residual oil was diluted with water and extracted with diethyl ether. Combined the organics, washed with brine, dried, filtered, and removed the solvent in vacuo leaving an orange oil which was purified via normal phase chromatography to leave 4-iodo-3-nitro-benzoic acid methyl ester (8.08 g, 77% yield) as a yellow solid.

¹H NMR (d6-DMSO) δ 8.37 (m, 1H), 8.28 (d, 1H, J=8.4 Hz), 7.88 (m, 1H), 3.89 (s, 3H). MS(TOF EI⁺) m/z 307 [M]⁺. IR (CHCl₃, cm⁻¹) 3026.8, 2955.4, 1729.9, 1599.7, 1539.9, 1437.7, 1290.2, 1243.9, 1119.5, 1023.1.

### c) 3-Amino-4-iodo-benzoic acid methyl ester

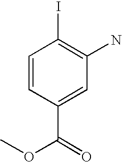

An ethyl acetate solution of 4-iodo-3-nitro-benzoic acid methyl ester (17.6 g, 57.32 mmol, 1 eq.) was hydrogenated over 5% sulfided platinum on carbon to give 3-amino-4-iodo-benzoic acid methyl ester (14.3 g, 90% yield).

MS(EI⁺) m/z 277 [M]⁺; 246 [M—OCH₃]⁺.

### d) 4-Iodo-3-methanesulfonylamino-benzoic acid methyl ester

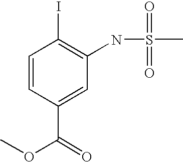

A pyridine solution of 3-amino-4-iodo-benzoic acid methyl ester (4.25 g, 15.34 mmol, 1 eq.) was treated with methanesulfonyl chloride as described for the synthesis of 3-iodo-4-methanesulfonylamino-benzoic acid methyl ester to give 4-iodo-3-methanesulfonylamino-benzoic acid methyl ester (5.16 g, 95% yield) as a tan solid.

¹H NMR (d6-DMSO) δ 8.20 (m, 1H), 7.93 (m, 1H), 7.73 (m, 1H), 3.88 (s, 3H), 3.65 (s, 3H). MS(ES⁻) m/z 354 [M−H]⁻. IR (KBr, cm⁻¹) 3014.8, 2936.5, 1718.9, 1588.9, 1436.5, 1370.8, 1290.4, 1248.2, 1159.2, 1115.2, 1008.6, 966.6, 927.3, 871.5, 760.7, 698.9. Analytical LC/MS: 100% (diode array detector).

### e) 2-Dimethylaminomethyl-1-methanesulfonyl-1H-indole-6-carboxylic acid methyl ester

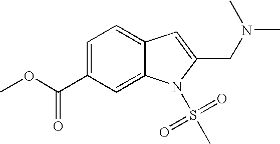

4-Iodo-3-methanesulfonylamino-benzoic acid methyl ester (4.75 g, .13.37 mmol, 1 eq.) was converted to the indole in the same was as described for 2-dimethylaminomethyl-1-methanesulfonyl-1H-indole-5-carboxylic acid methyl ester. After workup and normal phase chromatography, obtained 2-dimethylaminomethyl-1-methanesulfonyl-1H-indole-6-carboxylic acid methyl ester (3.02 g, 73% yield) as a yellow solid. Structural determination made on maleate salt.

¹H NMR (d6-DMSO) δ 8.59 (s, 1H), 7.89 (m, 2H), 7.18 (s, 1H), 6.07 (s, 2H), 4.45 (s, 2H), 3.91 (s, 3H), 3.59 (s, 3H), 2.71 (s, 6H). MS(ES⁺) m/z 311 [M+H]⁺; 266 [M−N(CH₃)₂]⁺. IR (KBr, cm⁻¹) 3408.6, 3039.2, 1717.0, 1621.6, 1575.9, 1476.6, 1370.4, 1299.9, 1205.9, 1174.2, 1160.4, 1091.2, 1046.8, 962.1, 866.8, 759.6. Analytical composition calculated for C₂₀H₂₂N₂O₈S C, 50.70; H, 5.20; N, 6.57. Found C, 50.36; H, 5.01; N, 6.50. Analytical LC/MS: 100% (diode array detector). M.P. softening at 158° C. and then 161-162° C.

### f) 2-Dimethylaminomethyl-1-methanesulfonyl-1H-indole-6-carboxylic acid hydrazide

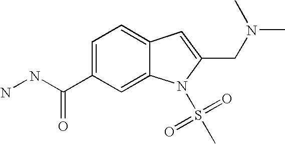

2-Dimethylaminomethyl-1-methanesulfonyl-1H-indole-6-carboxylic acid methyl ester (2.80 g, 9.02 mmol, 1 eq.) was converted to 2-dimethylaminomethyl-1-methanesulfonyl-1H-indole-6-carboxylic acid hydrazide in the same way as described for 2-dimethylaminomethyl-benzofuran-6-carboxylic acid hydrazide. Obtained 2-dimethylaminomethyl-1-methanesulfonyl-1H-indole-6-carboxylic acid hydrazide (2.42 g, 87% yield) as a yellow solid after workup and normal phase chromatography.

¹H NMR (d6-DMSO) δ 9.74 (s, 1H), 8.40 (m, 1H), 7.63 (m, 2H), 6.75 (s, 1H), 4.49 (s, 2H), 3.69 (s, 2H), 3.61 (s, 3H), 2.21 (s, 6H). MS(ES⁺) m/z 311 [M+H]⁺(KBr, cm⁻¹) 3305.4, 3149.1, 2947.0, 2823.4, 2782.6, 1630.7, 1606.3, 1523.3, 1471.1, 1375.4, 1355.0, 1330.6, 1292.9, 1169.7, 1141.4, 1052.7, 959.8, 771.1.Analytical composition calculated for C₁₃H₁₈N₄O₃S C, 50.31; H,.5.85; N, 18.05. Found C, 49.98; H, 5.54; N, 17.71. M.P. 170-173° C.

### g) 2-Dimethylaminomethyl-1-methanesulfonyl-1H-indole-6-carboxylic acid N′-[2-(2-phenoxy-ethylsulfanyl)-acetyl]-hydrazide

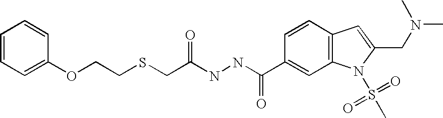

(2-Phenoxy-ethylsulfanyl)-acetic acid (1.45 g, 6.83 mmol, 1 eq.) was treated with EEDQ (2.53 g, 10.25 mmol, 1.5 eq.) and 2-dimethylaminomethyl-1-methanesulfonyl-1H-indole-6-carboxylic acid hydrazide (2.12 g, 6.83 mmol, 1 eq.) as described for 2-dimethylaminomethyl-benzofuran-6-carboxylic acid N′-[2-(2-phenoxy-ethylsulfanyl)-acetyl]-hydrazide to obtain 2-dimethylaminomethyl-1-methanesulfonyl-1H-indole-6-carboxylic acid N′-[2-(2-phenoxy-ethylsulfanyl)-acetyl]-hydrazide (3.20 g, 93% yield) as a yellow foam.

¹H NMR (d6-DMSO) δ 10.45 (s, 1H), 10.11 (s, 1H), 8.46 (s, 1H), 7.70 (m, 2H), 7.28 (m, 2H), 6.94 (m, 3H), 6.79 (s, 1H), 4.20 (t, 2H, J=6.6 Hz), 3.70 (s, 2H), 3.62 (s, 3H), 3.34 (s, 2H), 3.05 (t, 2H, J=6.6 Hz), 2.21 (s, 6H). MS(ES⁺) m/z 505 [M+H]⁺. IR (CHCl₃, cm⁻¹) 3237.3, 3019.0, 2949.1, 2823.1, 2777.6, 1635.2, 1497.6, 1456.7, 1367.8, 1287.7, 1243.2, 1171.9, 1053.4, 1020.7, 968.3, 852.0. Analytical composition calculated for C₂₃H₂₈N₄O₅S₂ C, 54.74; H, 5.59; N, 11.10. Found C, 55.00; H, 5.54; N, 10.92. M.P. softening at 57° C. and then 64-67° C.

### h) {1-Methanesulfonyl-6-[5-(2-phenoxy-ethylsulfanylmethyl)-[1,3,4]oxadiazol-2-yl]-1H-indol-2-ylmethyl}-dimethyl-amine

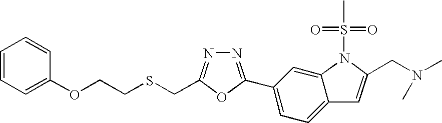

2-Dimethylaminomethyl-1-methanesulfonyl-1H-indole-6-carboxylic acid N′-[2-(2-15 phenoxy-ethylsulfanyl)-acetyl]-hydrazide (3.0 g, 5.94 mmol, 1 eq.) was converted to the oxadiazole as described for dimethyl-{6-[5-(2-phenoxy-ethylsulfanylmethyl)-[1,3,4]oxadiazol-2-yl]-benzofuran-2-ylmethyl}-amine. After workup and normal phase chromatography, obtained {1-methanesulfonyl-6-[5-(2-phenoxy-ethylsulfanylmethyl)-[1,3,4]oxadiazol-2-yl]-1H-indol-2-ylmethyl}-dimethyl-amine as a tan solid.

¹H NMR (d6-DMSO) δ 8.57 (s, 1H), 7.81 (m, 2H), 7.24 (m, 2H), 6.90 (m, 3H), 6.85 (s, 1H), 4.25 (s, 2H), 4.19 (t, 2H, J=6.6 Hz), 3.71 (s, 2H), 3.67 (s, 3H), 3.02 (t, 2H, J=6.6 Hz ), 2.23 (s, 6H). MS(ES⁺) m/z 487 [M+H]⁺. IR (KBr, cm⁻¹) 2942.1, 2824.0, 2769.7, 1584.9, 1561.7, 1464.9, 1359.0, 1290.4, 1228.4, 1171.2, 1052.1, 1027.3, 990.9, 837.3, 759.4. Analytical composition calculated for C₂₃H₂₆N₄O₄S₂ C, 56.77; H, 5.39; N, 11.51. Found C, 56.59; H, 5.31; N, 11.31. M.P. 127-129.5° C.

### Example 4

### Preparation of dimethyl-{6-[5-(2-phenoxy-ethylsulfanylmethyl)-[1,3,4]oxadiazol-2-yl]-1H-indol-2-ylmethyl }-amine

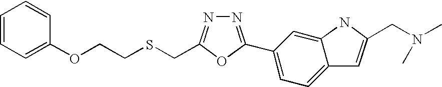

The sulfonyl group of {1-methanesulfonyl-6-[5-(2-phenoxy-ethylsulfanylmethyl)-[1,3,4]oxadiazol-2-yl]-1H-indol-2-ylmethyl}-dimethyl-amine (1.25 g, 2.57 mmol, 1 eq.) was removed in the same way as described for the synthesis of dimethyl-{5-[5-(2-phenoxy-ethylsulfanylmethyl)-[1,3,4]oxadiazol-2-yl]-1H-indol-2-ylmethyl }-amine. After workup and normal phase chromatography, obtained dimethyl-{6-[5-(2-phenoxy-ethylsulfanylmethyl)-[1,3,4]oxadiazol-2-yl]-1H-indol-2-ylmethyl}-amine (0.55 g, 52% yield) as a yellow crystalline solid.

¹H NMR (d6-DMSO) δ 11.46 (s, 1H), 7.93 (s, 1H), 7.57 (m, 2H), 7.24; (m, 2H), 6.91 (m, 3H), 6.39 (s, 1H), 4.22 (s, 2H), 4.19 (t, 2H, J=6.2 Hz), 3.59 (s, 2H), 3.02 (t, 2H, J=6.2 Hz), 2.20 (s, 6H). MS(ES⁺) m/z 409 [M+H]⁺; 364 [M−N(CH₃)₂]⁺. IR (KBr, cm⁻¹) 3230.3, 3061.9, 2945.7, 2912.2, 2816.2, 2766.5, 1602.7, 1587.1, 1554.9, 1499.3, 1456.6, 1405.6, 1355.2, 1250.3, 1212.8, 1173.5, 1032.7, 818.7, 752.9. Analytical LC/MS: 100% (diode array detector). M.P. 128-130° C.

### Example 5

### Preparation of dimethyl-{1-methyl-6-[5-(2-phenoxy-ethylsulfanylmethyl)-[1,3,4]oxadiazol-2-yl]-1H-indol-2-ylmethyl}-amine oxalate

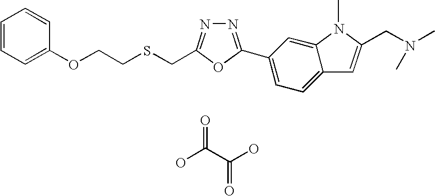

{1-Methanesulfonyl-6-[5-(2-phenoxy-ethylsulfanylmethyl)-[1,3,4]oxadiazol-2-yl]-1H-indol-2-ylmethyl}-dimethyl-amine was alkylated according to the procedure outlined in Tetrahedron Letters 1995, 36, 2029. Methanol (0.14 g, 0.17 mL, 4.31 mmol, 1.4 eq.) in toluene was treated with solid potassium carbonate (0.85 g, 6.16 mmol, 2eq.) and sodium hydride (0.15 g, 4.0 mmol, 1.3 eq.) and stirred at room temperature for 30 minutes. {1-Methanesulfonyl-6-[5-(2-phenoxy-ethylsulfanylmethyl)-[1,3,4]oxadiazol-2-yl]-1H-indol-2-ylmethyl}-dimethyl-amine was introduced as a suspension in toluene and the reaction stirred at 100° C. After 2 hours, the reaction was partitioned between ethyl acetate and water. The organic layer was collected and the solvent removed in vacuo leaving an reddish-brown solid which was purified by normal phase chromatography leaving an orange oil which was converted to the oxalate salt to give dimethyl-{1-methyl-6-[5-(2-phenoxy-ethylsulfanylmethyl)-[1,3,4]oxadiazol-2-yl]-1H-indol-2-ylmethyl}-amine oxalate (0.27 g, 17% yield) as a yellow solid.

¹H NMR (d6-DMSO) δ 8.07 (s, 1H), 7.69 (m, 2H), 7.24 (m, 2H), 6.91 (m, 3H), 6.69 (s, 1H), 4.21 (m, 6H), 3.86 (s, 3H), 3.03 (t, 2H, J=6.6 Hz), 2.58 (s, 6H). MS(ES⁺) m/z 423 [M+H]⁺; 378 [M−N(CH₃)₂]⁺. IR (KBr, cm⁻¹) 3442.3, 3037.8, 2928.1, 2710.6, 1724.0, 1600.2, 1558.6, 1496.0, 1470.6, 1408.1, 1354.6, 1240.8, 1172.7, 1016.2, 942.6, 824.0, 755.3, 706.5. Analytical composition calculated for C₂₅H₂₈N₄O₆S C, 58.58; H, 5.51; N, 10.93. Found C, 58.37; H, 5.43; N, 10.81. M.P. 125.5-129° C.

### Preparation of 5-[5-(2-Phenoxy-ethylsulfanylmethyl)-[1,3,4]oxadiazol-2-yl]-1H-indole

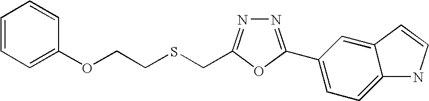

### a) 1H-Indole-5-carboxylic acid hydrazide

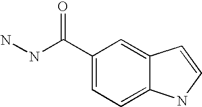

A DMF solution of indole-5-carboxylic acid (3.22 g, 20 mmol, 1 eq.) was treated with solid tetramethyl fluoroformamidinium hexafluorophosphate (5.28 g, 20 mmol, 1 eq.) and the solution cooled in an ice bath for 15 minutes. The triethylamine (4.05 g, 5.58 mL, 40 mmol, 2 eq.) was added via syringe followed by hydrazine hydrate (2.0 g, 40 mmol, 2 eq.). The ice bath was removed and the solution allowed to stir for 25 minutes. Added 100 mL of ice water and extracted with EtOAc. The organic layer was collected, dried, filtered, and removed the solvent in vacuo leaving an orange oil. The oil was purified by normal phase chromatography using 10% 2M NH₃ in MeOH in chloroform as the mobile phase to obtain 1H-indole-5-carboxylic acid hydrazide (0.85 g, 24% yield) as an orange crystalline solid.

¹H NMR (d6-DMSO) δ 11.29 (s, 1H), 9.55 (s, 1H), 8.09 (m, 1H), 7.59 (m, 1H), 7.40 (m, 2H), 6.51 (m, 1H), 4.41 (m, 2H). MS(ES⁺) m/z 176 [M+H]⁺. M.P. softening at 169° C. and then 173-177° C.

### b) 1H-Indole-5-carboxylic acid N′-[2-(2-phenoxy-ethylsulfanyl)-acetyl]-hydrazide

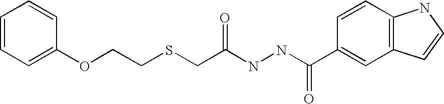

(2-Phenoxy-ethylsulfanyl)-acetic acid (3.63 g, 17.12 mmol, 1 eq.) was treated with EEDQ (4.66 g, 18.83 mmol, 1.1 eq.) and 1H-indole-5-carboxylic acid hydrazide (3.0 g, 17.12 mmol, 1 eq.) as described for 2-dimethylaminomethyl-benzofuran-6-carboxylic acid N′-[2-(2-phenoxy-ethylsulfanyl)-acetyl]-hydrazide to obtain 1H-indole-5-carboxylic acid N′-[2-(2-phenoxy-ethylsulfanyl)-acetyl]-hydrazide (5.20 g, 82% yield) as a yellow oil after normal phase chromatography using a step gradient of EtOAc in hexanes as the mobile phase.

MS(ES⁺) m/z 370 [M+H]⁺.

### c) 5-[5-(2-Phenoxy-ethylsulfanylmethyl)-[1,3,4]oxadiazol-2-yl]-1H-indole

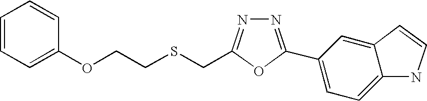

1H-Indole-5-carboxylic acid N′-[2-(2-phenoxy-ethylsulfanyl)-acetyl]-hydrazide (5.0 g, 13.53 mmol, 1 eq.) was cyclized to 5-[5-(2-Phenoxy-ethylsulfanylmethyl)-[1,3,4]oxadiazol-2-yl]-1H-indole in the same manner as described for dimethyl-{6-[5-(2-phenoxy-ethylsulfanylmethyl)-[1,3,4]oxadiazol-2-yl]-benzofuran-2-ylmethyl}-amine above. Obtained the product (1.85 g, 39% yield) as a white solid after normal phase chromatography.

¹H NMR (d6-DMSO) δ 11.51 (s, 1H), 8.19 (s, 1H), 7.71 (m, 1H), 7.56 (m, 1H), 7.50 (m, 1H), 7.27 (m, 2H), 6.93 (m, 3H), 6.60 (s, 1H), 4.21 (m, 4H), 3.04 (t, 2H, J=6.6 Hz). MS(ES⁺) m/z 352 [M+H]⁺. IR (CHCI₃, cm⁻¹) 3475.8, 2998.8, 1619.9, 1600.7, 1587.4, 1558.8, 1497.6, 1463.6, 1450.8, 1243.8. Analytical LC/MS: 10Q% (diode array detector). M.P. 130-132° C.

### Example 6

### Preparation of dimethyl-{5-[5-(2-phenoxy-ethylsulfanylmethyl)-[1,3,4]oxadiazol-2-yl]-1H-indol-3-ylmethyl}-amine oxalate

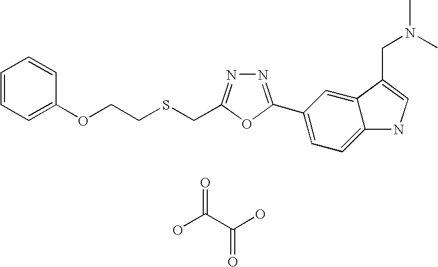

A dichloroethane solution of 5-[5-(2-phenoxy-ethylsulfanylmethyl)-[1,3,4]oxadiazol-2-yl]-1H-indole (1.47 g, 4.18 mmol, 1 eq.) was treated with N,N-dimethylmethyleneammonium chloride (0.47 g, 5.02 mmol, 1.2 eq.) as a solid and the solution heated to 80° C. When the reaction was complete, diluted the reaction with dichloromethane and washed with aqueous 1M NaOH and brine. The organic layer was collected, dried, filtered, and the solvent removed leaving an orange oil in the flask that was purified via normal phase chromatography using 10% 2M NH₃ in methanol in Et₂O as the mobile phase. Removal of the solvent left dimethyl-{5-[5-(2-phenoxy-ethylsulfanylmethyl)-[1,3,4]oxadiazol-2-yl]-1H-indol-3-ylmethyl}-amine (1.64 g, 96% yield) as a yellow oil.

The oil was converted to the oxalate salt by adding 1.1 eq. of oxalic acid in ethyl acetate to an ethyl acetate solution of the amine. The resulting white solid was recrystallized from methanol leaving dimethyl-{5-[5-(2-phenoxy-ethylsulfanylmethyl)-[1,3,4]oxadiazol-2-yl]-1H-indol-3-ylmethyl}-amine oxalate as an off-white solid.

¹H NMR (d6-DMSO) δ 11.91 (s, 1H), 8.49 (s, 1H), 7.78 (m, 1H), 7.70 (s, 1H), 7.62 (m, 1H), 7.27 (m, 2H), 6.93 (m, 3H), 4.46 (s, 2H), 4.20 (m, 4H), 3.03 (t, 2H, J=6.6 Hz), 2.71 (s, 6H). MS(ES⁺) m/z 409 [M+H]⁺; 364 [M−N(CH₃)₂]+. IR (KBr, cm⁻¹) 3218.7 3027.7, 1722.5, 1621.3, 1601.4, 1585.9, 1554.7, 1489.6, 1465.9, 1454.9, 1251.5, 758.4. Analytical LC/MS: 100% (diode array detector). M.P. 159-163° C.

### Preparation of 6-[5-(2-Phenoxy-ethylsulfanylmethyl)-[1,3,4]oxadiazol-2-yl]-1H-indole

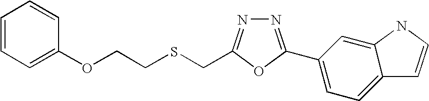

### a) 1H-Indole-6-carboxylic acid hydrazide

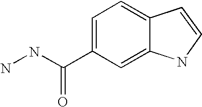

Indole-6-carboxylic acid (7.4 g, 45.92 nmmol, 1 eq.) was converted to 1H-indole-6-carboxylic acid hydrazide in the same way as described for 1H-indole-5-carboxylic acid hydrazide to obtain 1H-indole-6-carboxylic acid hydrazide (3.29 g, 41%) as a tan solid after workup and purification.

MS(ES⁺) m/z 176 [M+H]⁺.

### b) 1H-Indole-6-carboxylic acid N′-[2-(2-phenoxy-ethylsulfanyl)-acetyl]-hydrazide

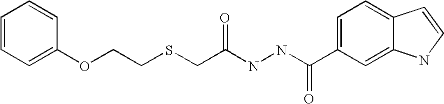

(2-Phenoxy-ethylsulfanyl)-acetic acid (3.88 g, 18.27 mmol, 1 eq.) was treated with EEDQ (4.97 g, 20.10 mmol, 1.1 eq.) and 1H-indole-6-carboxylic acid hydrazide (3.2 g, 18.27 mmol, 1 eq.) as described for 2-dimethylaminomethyl-benzofuran-6-carboxylic acid N′-[2-(2-phenoxy-ethylsulfanyl)-acetyl]-hydrazide to obtain 1H-indole-6-carboxylic acid N′-[2-(2-phenoxy-ethylsulfanyl)-acetyl]-hydrazide (4.75 g, 70% yield) as a yellow oil after normal phase chromatography using a step gradient of EtOAc in hexanes as the mobile phase.

MS(ES⁺) m/z 370 [M+H]⁺.

### c) 6-[5-(2-Phenoxy-ethylsulfanylmethyl)-[1,3,4]oxadiazol-2-yl]-1H-indole

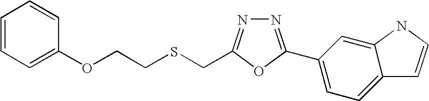

1H-Indole-6-carboxylic acid N′-[2-(2-phenoxy-ethylsulfanyl)-acetyl]-hydrazide (4.7 g, 12.72 mmol, 1 eq.) was cyclized to 6-[5-(2-phenoxy-ethylsulfanylmethyl) -[1,3,4]oxadiazol-2-yl]-1H-indole in the same manner as described for dimethyl-{6-[5-(2-phenoxy-ethylsulfanylmethyl)-[1 ,3,4]oxadiazol-2-yl]-benzofuran-2-ylmethyl}-amine. Obtained the product (3.12 g, 70% yield) as a brown solid after normal phase chromatography and recrystallization from ethyl acetate.

¹H NMR (d6-DMSO) δ 8 11.50 (s, 1H), 8.04 (s, 1H), 7.72 (m, 1H), 7.61 (m, 2H), 7.26 (m, 2H), 6.92 (m, 3H), 6.56 (s, 1H), 4.20 (m, 4H), 3.04 (t, 2H, J=6.6 Hz). MS(ES⁺) m/z 352 [M+H]⁺. MS(ES⁻) m/z 350 [M-H]⁻. IR (KBr, cm⁻¹) 3456.1, 3275.6, 2984.6, 2926.1, 2878.0, 1728.9, 1602.8, 1585.2, 1553.9, 1499.2, 1356.5, 1245.0, 1173.6, 1035.9, 822.2,755.9, 730.4. Analytical LC/MS: 100% (diode array detector). M.P. 162-164° C.

### Example 7

### Preparation of Dimethyl-{6-[5-(2-phenoxy-ethylsulfanylmethyl)-[1,3,4]oxadiazol-2-yl]-1H-indol-3-ylmethyl}-amine maleate

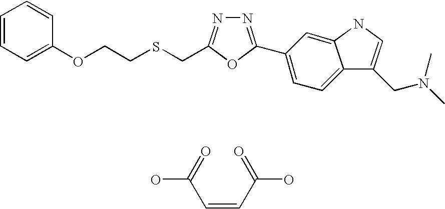

6-[5-(2-Phenoxy-ethylsulfanylmethyl)-[1,3,4]oxadiazol-2-yl]-1H-indole (2.0 g, 5.69 mmol, 1 eq.) was converted to dimethyl-{6-[5-(2-phenoxy-ethylsulfanylmethyl) -[1,3,4]oxadiazol-2-yl]-1H-indol-3-ylmethyl}-amine in the same fashion as described for dimethyl-{5-[5-(2-phenoxy-ethylsulfanylmethyl)-[1,3,4]oxadiazol-2-yl]-1H-indol-3-ylmethyl ) -amine. Obtained the product as a yellow solid after workup and normal phase chromatography. Converted to the maleate salt by addition of maleic acid in ethyl acetate to obtain dimethyl-{16-[5-(2-phenoxy-ethylsulfanylmethyl)-[1,3,4]oxadiazol-2-yl]-1H -indol-3-ylmethyl}-amine maleate (0.6912 g, 23% yield) as an orange solid.

¹H NMR (d6-DMSO) δ 11.85 (s, 1H), 8.09 (s, 1H), 7.97 (m, 1H), 7.75 (m, 2H), 7.26 (m, 2H), 6.92 (m, 3H), 6.02 (s, 2H), 4.47 (s, 2H), 4.25 (s, 2H), 4.20 (t, 2H, J=6.6 Hz), 3.04 (t, 2H, J=6.6 Hz), 2.76 (s, 6H). MS(ES⁺) m/z 409 [M+H]⁺. MS(ES³¹ ) m/z 407 [M-H]³¹ . IR (KBr, cm^{31 1}) 3126.8, 3057.2, 2997.1, 2943.9, 2912.4, 1624.2, 1585.5, 1490.4, 1463.9, 1427.8, 1357.4, 1238.7, 1118.7, 1063.5, 973.9, 927.8, 860.6, 757.2, 725.2. Analytical LC/MS: 91% (diode array detector). M.P. gradual darkening as heated and melting at 127-129° C.

### Preparation of 1-Methyl-5-[5-(2-phenoxy-ethylsulfanylmethyl)-[1,3,4]oxadiazol-2-yl]-1H-indole

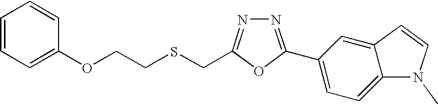

### a) 1-Methyl-1H-indole-5-carboxylic acid methyl ester

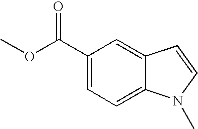

A DMF solution of methyl indole-5-carboxylate (5.0 g, 28.54 mmol, 1 eq.) was cooled in an ice bath and then treated with sodium hydride (1.37 g, 34.29 mmol, 1.2 eq.). After 20 minutes at 0° C., the reaction was treated with iodomethane (6.08 g, 2.7 mL, 42.81 mmol, 1.5 eq.). After stirring for 4 hours, the reaction was quenched with water. The reaction was extracted twice with 150 mL of ethyl acetate and the organic layers were combined, dried, filtered, and the solvent removed in vacuo leaving a yellow oil that was triturated with hexane to obtain 1-methyl-1H-indole-5-carboxylic acid methyl ester (5.22 g, 97% yield) as a tan solid.

¹H NMR (d6-DMSO) δ 8.26 (s, 1H), 7.77 (m, 1H), 7.52 (m, 1H), 7.45 (m, 1H), 6.59 (m, 1H), 3.84 (s, 3H), 3.83 (s, 3H). MS(ES⁺) m/z 190 [M+H]⁺; 158 [M-OCH₃]⁺. IR (CHCl₃, cm⁻¹) 3019.1, 2951.8, 1707.6, 1614.6, 1515.9, 1437.6, 1344.7, 1309.9, 1268.6, 1197.1, 1108.5, 1082.8. Analytical composition calculated for C₁₁H₁₁NO₂ C, 69.83; H, 5.86; N, 7.40. Found C, 69.85; H, 5.73; N, 7.36. M.P. 108-110° C.

### b) 1-Methyl-1H-indole-5-carboxylic acid hydrazide

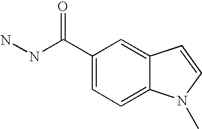

1-Methyl-1H-indole-5-carboxylic acid methyl ester (4.5 g, 23.78 mmol, 1 eq.) was converted-to 1-methyl-1H-indole-5-carboxylic acid hydrazide in the same way as described for 2-dimethylaminomethyl-benzofuran-6-carboxylic acid hydrazide. Obtained the product as a pink solid that was recrystallized from dichloromethane to give 1-methyl -1H-indole-5-carboxylic acid hydrazide (3.46 g, 77% yield) as an off-white solid.

¹H NMR (d6-DMSO) δ 9.58 (s, 1H), 8.09 (m, 1H), 7.66 (m, 1H), 7.45 (m, 1H), 7.38 (m, 1H), 6.51 (m, 1H), 4.41 (m, 2H), 3.81 (s, 3H). MS(ES⁺) m/z 190 [M+H]⁺. IR (KBr, cm⁻¹) 3312.9, 1662.2, 1610.1, 1540.7, 1487.2, 1341.8, 1245.5, 1145.7, 1074.5, 963.5, 894.9, 735.7, 720.8, 683.1. Analytical composition calculated for C₁₀H₁₁N₃O C, 63.48; H, 5.86; N, 22.21. Found C, 63.48; H, 5.81; N, 22.24. M.P. 140-141° C.

### c) 1-Methyl-1H-indole-5-carboxylic acid N′-[2-(2-phenoxy-ethylsulfanyl)-acetyl]-hydrazide

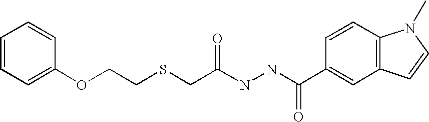

(2-Phenoxy-ethylsulfanyl)-acetic acid (3.36 g, 15.85 mmol, 1 eq.) was treated with EEDQ (5.88 g, 23.78 mmol, 1.5 eq.) and 1-methyl-1H-indole-5-carboxylic acid hydrazide (3.0 g, 15;85 mmol, 1 eq.) as described for 2-dimethylamino methyl-benzofuran-6-carboxylic acid N′-[2-(2-phenoxy-ethylsulfanyl)-acetyl]-hydrazide to obtain 1-methyl-1H -indole-5-carboxylic acid N′-[2-(2-phenoxy-ethylsulfanyl)-acetyl]-hydrazide (3.54 g, 58% yield) as a yellow solid after normal phase chromatography using a step gradient of EtOAc in hexanes as the mobile phase.

¹H NMR (d6-DMSO) δ 10.26 (s, 1H), 10.03 (s, 1H), 8.17 (s, 1H), 7.71 (m, 1H), 7.50 (m, 1H), 7.42 (m, 1H), 7.28 (m, 2H), 6.96 (m, 3H), 6.55 (m, 1H), 4.21 (t, 2H, J=6.6 Hz), 3.83 (s, 3H), 3.33 (s, 2H), 3.06 (t, 2H, J=6.6 Hz). MS(ES⁺) m/z 384 [M+H ]⁺. IR (KBr, cm⁻¹) 3292.1, 3218.8, 3006.0, 1688.0, 1631.2, 1598.9, 1515.6, 1486.12, 1329.5, 1281.5, 1251.1, 1169.5, 1113.7, 1031.4, 887.7, 750.7, 722.8, 690.6. Analytical composition calculated for C₂₀H₂₁N₃O₃S C, 62.64; H, 5.52; N, 10.96. Found C, 62.39; H, 5.18; N, 10.82. M.P. 134-137° C.

### d) 1-Methyl-5-[5-(2-phenoxy-ethylsulfanylmethyl)-[1,3,4]oxadiazol-2-yl]-1H-indole

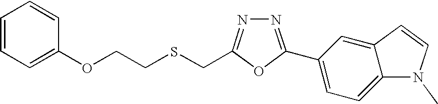

1-Methyl-1H-indole-5-carboxylic acid N′-[2-(2-phenoxy-ethylsulfanyl)-acetyl]-hydrazide (3.2 g, 8.34 mmol, 1 eq.) was cyclized to 1-methyl-5-[5-(2-phenoxy -ethylsulfanylmethyl)-[1,3,4]oxadiazol-2-yl]-1H-indole in the same manner as described for dimethyl-{6-[5-(2-phenoxy-ethylsulfanylmethyl)-[1,3,4]oxadiazol-2-yl]-benzofuran-2-ylmethyl}-amine. Obtained the product (2.28 g, 75% yield) as a yellow solid after normal phase chromatography.

₁H NMR (d6-DMSO) δ 8.18 (m, 1H), 7.76 (m, 1H), 7.62 (m, 1H), 7.47 (m, 1H), 7.26 (m, 2H), 6.92 (m, 3H), 6.59 (m, 1H), 4.20 (m, 4H), 3.85 (s, 3H), 3.04 (t, 2H, J=6.6 Hz). MS(ES⁺) m/z 366 [M+H]⁺¹. IR (CHCl₃, cm₁) 3009.1, 1601.1, 1588.0, 1558.2, 1497.5, 1480.2, 1423.6, 1343.1, 1295.2, 1244.9, 1223.4, 1212.1, 1173.2, 1081.6, 1033.6. Analytical composition calculated for C₂₀H₁₉N₃O₂S C, 65.73; H, 5.24; N, 11.50. Found C, 65.34; H, 5.12; N, 11.29. M.P. 136-139° C.

### Example 8

### Preparation of dimethyl-{1-methyl-5-[5-(2-phenoxy-ethylsulfanylmethyl) -[1,3,4]oxadiazol-2-yl]-1H-indol-3-ylmethyl}-amine oxalate

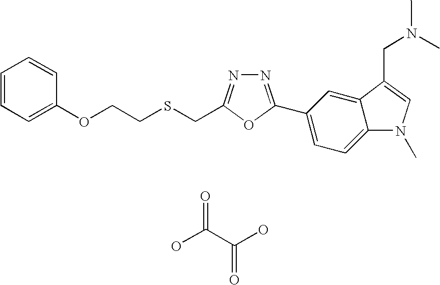

1-Methyl-5-[5-(2-phenoxy-ethylsulfanylmethyl)-[1,3,4]oxadiazol-2-yl]-1H-indole (2.0 g, 5.47 mmol, 1 eq.) was converted to dimethyl-(1-methyl-5-[5-(2-phenoxy -ethylsulfanylmethyl)-[1,3,4]oxadiazol-2-yl]-1H-indol-3-ylmethyl}-amine in the same fashion as described for dimethyl-{5-[5-(2-phenoxy-ethylsulfanylmethyl) -[1,3,4]oxadiazol-2-yl]-1H-indol-3-ylmethyl}-amine. Obtained dimethyl-{1-methyl-5-[5-(2-phenoxy-ethylsulfanylmethyl)-[1,3,4]oxadiazol-2-yl]-1H-indol-3-ylmethyl}-amine as a yellow oil after workup and normal phase chromatography. Converted to the oxalate salt by addition of oxalic acid in acetone to obtain dimethyl-{1-methyl-5-[5-(2-phenoxy -ethylsulfanylmethyl)-[1,3,4]oxadiazol-2-yl]-1H-indol-3-ylmethyl}-amine oxalate (0.45 g, 16% yield) as an off-white solid.

₁H NMR (d6-DMSO) δ 8.49 (s, 1H), 7.83 (m, 1H), 7.69 (m, 2H), 7.25 (m, 2H), 10 6.92 (m, 3H), 4.43 (s, 2H), 4.23 (s, 2H), 4.20 (t, 2H, J=6.6 Hz), 3.89 (s, 3H), 3.03 (t, 2H, J=6.6 Hz), 2.71 (s, 6H). MS(ES⁺) m/z 423 [M+H]⁺; 378 [M−N(CH₃)₂]⁺. IR KBr, cm⁻¹) 3041.0, 2938.1, 2704.3, 1721.4, 1622.8, 1601.3, 1557.5, 1483.0, 1393.9, 1307.6, 1247.8, 1204.9, 1083.4, 1057.5, 1011.2, 929.0, 803.2, 753.7, 702.8. Analytical composition calculated for C₂₅H₂₈N₄O₆S C, 58.58; H, 5.51; N, 10.93. Found C, 58.41; H, 5.38; N, 10.81. M.P. 151-154° C.

### Preparation of 4-Dimethylamino-naphthalene-1-carboxylic acid N′-[2-(2-phenoxy -ethylsulfanyl)-acetyl]-hydrazide

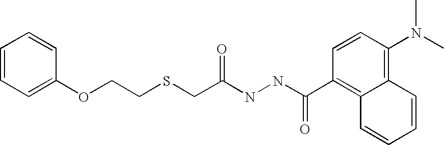

### a) 4-Dimethylamino-naphthalene-1-carboxylic acid hydrazide

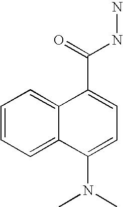

4-Dimethylaminonaphthalene-1-carboxylic acid (5.05 g, 23.46 mmol, 1 eq.) was converted to 4-dimethylamino-naphthalene-1-carboxylic acid hydrazide in a similar manner to that described for 1H-indole-5-carboxylic acid hydrazide. Obtained 4-dimethylamino-naphthalene-1-carboxylic acid hydrazide as an orange oil after normal phase chromatography using 10% 2M NH₃ in methanol in diethyl ether as the mobile phase.

MS(ES⁺) m/z 230 [M+H]⁺.

### b) 4-Dimethylamino-naphthalene-1-carboxylic acid N′-[2-(2-phenoxy-ethylsulfanyl) -acetyl]-hydrazide

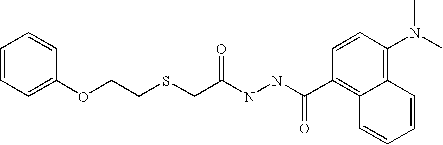

(2-Phenoxy-ethylsulfanyl)-acetic acid (2.74 g, 12.91 mmol, 1 eq.) was treated with EEDQ (3.51 g, 14.20 mmol, 1.1 eq.) and 4-dimethylamino-naphthalene-1-carboxylic acid hydrazide (2.96 g, 12.91 mmol, 1 eq.) as described for 2-dimethylaminomethyl -benzofuran-6-carboxylic acid N′-[2-(2-phenoxy-ethylsulfanyl)-acetyl]-hydrazide to obtain 4-dimethylamino-naphthalene-1-carboxylic acid N′-[2-(2-phenoxy-ethylsulfanyl)-acetyl]-hydrazide (4.09 g, 75% yield) as a yellow oil after workup of the reaction.

¹H NMR (d6-DMSO) δ 10.27 (s, 1H), 10.17 (s, 1H), 8.36 (m, 1H), 8.18, (m, 1H), 7.57 (m, 3H), 7.29 (m, 2H), 7.11 (m, 1H), 6.96 (m, 3H), 4.22 (t, 2H, J=6.6 Hz), 3.36 (s, 2H), 3.07 (t, 2H, J=6.6 Hz), 2.88 (s, 6H). MS(ES⁺) m/z 424 [M+H]⁺.

### Example 9

### Preparation of dimethyl-{4-[5-(2-phenoxy-ethylsulfanylmethyl)-[1,3,4]oxadiazol-2-yl]-naphthalen-1-yl}-arilne

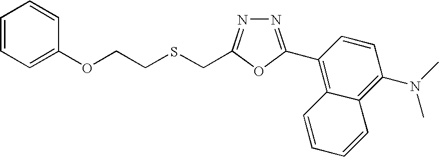

4-Dimethylamino-naphthalene-1-carboxylic acid N′-[2-(2-phenoxy-ethylsulfanyl) -acetyl]-hydrazide (4.0 g, 9.44 mmol, 1 eq.) was cyclized in a similar manner to that described for dimethyl-{6-[5-(2-phenoxy-ethylsulfanylmethyl)-[1,3,4]oxadiazol-2-yl]benzofuran-2-ylmethyl}-amine. Obtained a dark brown oil after removal of the solvent that was purified by normal phase chromatography using 15% ethyl acetate in hexanes as the mobile phase. Removal of the solvent left dimethyl-{4-[5-(2-phenoxy -ethylsulfanylmethyl)-[1,3,4]oxadiazol-2-yl]-naphthalen-1-yl}-amine (2.44 g, 64% yield) as a brown oil.

₁H NMR (d6-DMSO) □ 9.09 (d, 1H, J=8.1 Hz), 8.25 (d, 1H, J=8.8 Hz), 8.03 (m, 1H), 7.65 (m, 2H), 7.23 (m, 3H), 6.91 (m, 3H), 4.28 (s, 2H), 4.21 (t, 2H, J=6.6 Hz), 3.06 (t, 2H, J=6.6 Hz), 2.94 (s, 6H). MS(ES⁺) m/z 406 [M+H]⁺. IR (CHCl₃, cm⁻¹) 3010.2, 2946.8, 2838.8, 1600.7, 1577.1, 1530.2, 1497.6, 1455.8, 1400.3, 1243.6, 1030.8, 750.4, 672.0. Analytical LC/MS: 100% (diode array detector).

### Preparation of {6-[5-(2-Phenoxy-ethylsulfanylmethyl)-[1,3,4]oxadiazol-2-yl]-naphthalen -2-yl}-methanol

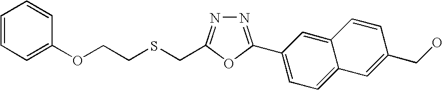

### a) (6-Bromo-naphthalen-2-yl)-methanol

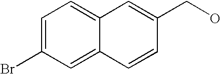

A THF solution of 6-bromo-2-naphthoic acid (3.5 g, 13.94 mmol, 1 eq.) was cooled to 0° C. in an ice bath and then treated dropwise with borane-THF complex (1 M solution in THF, 16.7 mL, 16.7 mmol, 1.2 eq.) via syringe. After addition was complete, the reaction was allowed to gradually warm to room temperature and stir at that temperature overnight. Several milliliters of water were added to the reaction to quench the remainder of the borane. The reaction was diluted with water and extracted with ethyl acetate. The organic layer was separated and then washed with aqueous 1 M NaOH and then brine. The organic layer was then collected, dried over MgSO₄, filtered and the solvent removed in vacuo leaving (6-bromo-naphthalen-2-yl)-methanol (2.56 g, 77% yield) as a white solid in the flask.

¹H NMR (d6-DMSO) δ 8.17 (m, 1H), 7.87 (m, 3H), 7.60 (m, 1H), 7.51 (m, 1H), 5.36 (t, 1H, J=5.9 Hz), 4.65 (m, 2H). MS(TOF EI⁺) m/z 236, 238 [M]⁺.

### b) (6-Bromo-naphthalen-2-ylmethoxy)-tert-butyl-dimethyl-silane

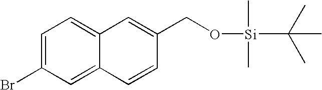

A CH₂Cl₂ solution of (6-bromo-naphthalen-2-yl)-methanol (0.88 g, 3.71 mmol, 1 eq.) and imidazole (0.51 g, 7.42 mmol, 2 eq.) was treated with tert-butyldimethylsilyl chloride (0.61 g, 4.08 mmol, 1.1 eq.) and the solution allowed to stir at room temperature. After 4 hours, the reaction was washed with water and then brine. The organic layer was collected, dried over MgSO₄, filtered, and the solvent removed in vacuo leaving (6-bromo-naphthalen-2-ylmethoxy)-tert-butyl-dimethyl-silane (1.42 g, contains some tert -butyldimethylsilyl chloride) as a white semi-solid.

¹H NMR (d6-DMSO) δ 8.07 (m, 1H), 7.77 (m, 3H), 7.50 (m, 1H), 7.39 (m, 1H), 4.75 (s, 2H), 0.82 (s, 9H), 0.00 (s, 6H). MS(FAB+) m/z 349, 351 [M-H]⁺.

### c) 6-(tert-Butyl-dimethyl-silanyloxymethyl)-naphthalene-2-carboxylic acid methyl ester

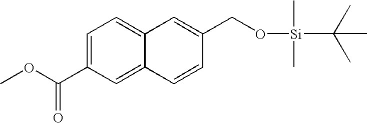

A DMSO solution of (6-bromo-naphthalen-2-ylmethoxy)-tert-butyl-dimethyl -silane (3.78 g, 10.75 mmol, 1 eq.) was treated with palladium acetate (0.24 g, 1.08 mmol, 10 mol%), triphenylphosphine (0.57 g, 2.16 mmol, 20 mol%), triethylamine (2.5 mL), and methanol (5 mL). The flask was evacuated and filled with carbon monoxide. The reaction was heated to 85° C. and stirred at that temperature overnight under a CO balloon. The reaction was diluted with diethyl ether and washed twice with water and then brine. The organic layer was collected, dried over MgSO₄, filtered, and the solvent removed in vacuo leaving a brown oil which was purified by normal phase chromatography using a step gradient of ethyl acetate in hexanes leaving 6-(tert-butyl-dimethyl-silanyloxymethyl) -naphthalene-2-carboxylic acid methyl ester (4.17 g, 87% yield) as a white solid.

¹H NMR (d6-DMSO) δ 8.49 (s, 1H), 7.98 (m, 1H), 7.86 (m, 2H), 7.78 (s, 1H), 7.42 (m, 1H), 4.79 (s, 2H), 3.80 (s, 3H), 0.81 (s, 9H), 0.00 (s, 6H). MS(TOF EI⁺) m/z 330 [M]⁺. IR (CHCI₃, cm⁻¹) 2954.8, 2930.3, 2857.7, 1716.8, 1437.0, 1283.4, 1253.9, 1239.8, 1199.2, 1127.7, 1097.9, 839.4. Analytical composition calculated for C₁₉H₂₆O₃Si C, 69.05; H, 7.93; N, 0. Found C, 69.07; H, 7.99; N, 0.05. M.P. 51-54° C.

### d) 6-(tert-Butyl-dimethyl-silanyloxymethyl)-naphthalene-2-carboxylic acid hydrazide

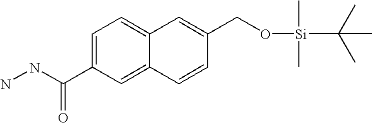

An ethanolic suspension of 6-(tert-butyl-dimethyl-silanyloxymethyl)-naphthalene-2-carboxylic acid methyl ester (3.53 g, 10.68 mmol, 1 eq.) was treated with hydrazine (1.71 g, 53.40 mmol, 1.68 mL, 5 eq.) and the reaction stirred at reflux. When the reaction was complete, the reaction was allowed to cool and the white suspension treated with water and the white solid collected by filtration and washed with water. The solid was dried under vacuum at room temperature leaving 6-(tert-butyl-dimethyl -silanyloxymethyl)-naphthalene-2-carboxylic acid hydrazide as a white solid (3.092 g, 88% yield).

¹H NMR (d6-DMSO) δ 9.89 (s, 1H), 8.41 (m, 1H), 7.93 (m, 4H), 7.51 (m, 1H), 4.89 (s, 2H), 4.54 (s, 2H), 0.93 (s, 9H), 0.11 (s, 6H). MS(ES⁺) m/z 331 [M+H]⁺. IR (KBr, cm⁻¹) 3311.3, 3256.6, 3196.8, 2955.3, 2892.6, 2856.1, 1653.8, 1626.4, 1538.3, 1253.8, 1087.7 842.9, 776.9. Analytical composition calculated for C₁₈H₂₆N₂O₂Si C, 65.42; H, 7.93, N, 8.48. Found C, 65.42; H, 7.93; N, 8.47. Found C, 65.80; H, 8.06; N, 8.01.

### e) 6-(tert-Butyl-dimethyl-silanyloxymethyl)-naphthalene-2-carboxylic acid N′-[2-(2-phenoxy-ethylsulfanyl)-acetyl]-hydrazide

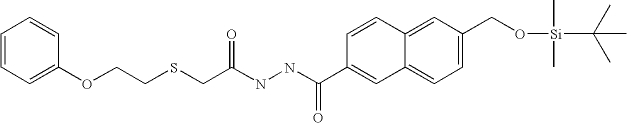

(2-Phenoxy-ethylsulfanyl)-acetic acid (1.86 g, 8.77 mmol, 1 eq.) was treated with EEDQ (2.39 g, 9.65 mmol, 1.1 eq.) and 6-(tert-butyl-dimethyl-silanyloxymethyl) -naphthalene-2-carboxylic acid hydrazide (2.90 g, 8.77 mmol, 1 eq.) as described for 2-dimethylaminomethyl-benzofuran-6-carboxylic acid N′-[2-(2-phenoxy-ethylsulfanyl) -acetyl]-hydrazide to obtain 6-(tert-butyl-dimethyl-silanyloxymethyl)-naphthalene-2-carboxylic acid N′-[2-(2-phenoxy-ethylsulfanyl)-acetyl]-hydrazide (3.6 g, 78% yield) as a white solid.

¹H NMR (d6-DMSO) δ 10.46 (s, 1H), 10.05 (s, 1H), 8.36 (s, 1H), 7.83 (m, 4H), 7.41 (m, 1H), 7.17 (m, 2H), 6.83 (m, 3H), 4.78 (s, 2H), 4.09 (t, 2H, J=6.6 Hz), 3.19 (s, 2H), 2.94 (t, 2H, J=6.6 Hz), 0.81 (s, 9H), 0.00 (s, 6H). MS(ES^{) m/z} 525 [M+H]⁺. IR (KBr, cm⁻¹) 3222.2, 3018.7, 2956.4, 2927.5, 2857.1, 1681.0, 1641.3, 1524.2, 1496.9, 1299.8, 1245.4, 1096.9, 855.7, 776.7, 753.0, 690.4. Analytical composition calculated for C₂₈H₃₆N₂O₄SSi C, 64.09; H, 6.92; N, 5.34. Found C, 63.75; H, 6.46; N, 5.30. M.P. 124-126° C.

### f) 2-[6-(tert-Butyl-dimethyl-silanyloxymethyl)-naphthalen-2-yl]-5-(2-phenoxy -ethylsulfanylmethyl)-[1,3,4]oxadiazole

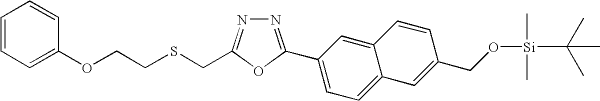

6-(tert-Butyl-dimethyl-silanyloxymethyl)-naphthalene-2-carboxylic acid N′-[2-(2-phenoxy-ethylsulfanyl)-acetyl]-hydrazide (3.28 g, 6.25 mmol, 1 eq.) was cyclized in a similar manner to that described for dimethyl-{6-[5-(2-phenoxy-ethylsulfanylmethyl) -[1,3,4]oxadiazol-2-yl]-benzofuran-2-ylmethyl}-amine. Obtained a brown oil after workup that was purified by normal phase chromatography using a step gradient of ethyl acetate in hexanes to give 2-[6-(tert-butyl-dimethyl-silanyloxymethyl)-naphthalen-2-yl]-5-(2-phenoxy-ethylsulfanylmethyl)-[1,3,4]oxadiazole (3.14 g, 99% yield) as abrown solid.

¹H NMR (d6-DMSO) δ 8.55 (s, 1H), 8.08 (m, 3H), 7.92 (s, 1H), 7.56 (m, 1H), 7.25 (m, 2H), 6.92 (m, 3H), 4.91 (s, 2H), 4.27 (s, 2H), 4.21 (t, 2H, J=6.6 Hz), 3.05 (t, 2H, J=6.6 Hz), 0.94 (s, 9H), 0.13 (s, 6H). MS(ES⁺) m/z 507 [M+H]⁺. IR (KBr, cm⁻¹) 3442.5, 2953.4, 2926.9, 2855.9, 1602.8, 1585.2, 1558.4, 1499.7, 1461.7, 1256.1, 1066.6, 853.2, 841.5, 776.3. Analytical composition calculated for C₂₈H₃₄N₂O₃SSi C, 66.37; H, 6.76; N, 5.53. Found C, 66.36; H, 6.76; N, 5.66.

### g) {6-[5-(2-Phenoxy-ethylsulfanylmethyl)-[1,3,4]oxadiazol-2-yl]-naphthalen-2-yl }-methanol

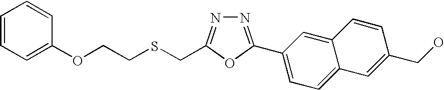

A methanolic solution of 2-[6-(tert-butyl-dimethyl-silanyloxymethyl)-naphthalen -5 2-yl]-5-(2-phenoxy-ethylsulfanylmethyl)-[1,3,4]oxadiazole (3.0 g, 5.92 mmol, 1 eq.) was treated with p-toluenesulfonic acid monohydrate (0.06 g, 2% of starting material). When the reaction complete, the now yellow suspension was concentrated in vacuo and the white solid was collected by filtration leaving {6-[5-(2-phenoxy-ethylsulfanylmethyl) -[1,3,4]oxadiazol-2-yl]-naphthalen-2-yl}-methanol (2.18 g, 94% yield).

¹H NMR (d6-DMSO) δ 8.55 (s, 1H), 8.05 (m, 3H), 7.94 (s, 1H), 7.58 (m, 1H), 7.26 (m, 2H), 6.93 (m, 3H), 5.42 (t, 1H, J=5.5 Hz), 4.71 (d, 2H, J=5.5 Hz), 4.27 (s, 2H), 4.21 (t, 2H, J=6.6 Hz), 3.06 (t, 2H, J=6.6 Hz). MS(ES⁺) m/z 393 [M+H]⁺. IR (KBr, cm⁻¹) 3381.9, 2980.7, 2930.4, 2866.2, 1602.0, 1572.3, 1546.5, 1498.3, 1464.2, 1385.6, 1252.4, 1157.2, 1045.3, 883.1, 823.8, 753.0, 694.2. Analytical composition calculated for C₂₂H₂₀N₂O₃S C, 67.33; H, 5.14; N, 7.14. Found C, 67.18; H, 4.99; N, 7.12. M.P. 132-133° C.

### Example 10

### Preparation of 2-(6-chloromethyl-naphthalen-2-yl)-5-(2-phenoxy-ethylsulfanylmethyl) -[1,3,4]oxadiazole and methanesulfonic acid 6-[5-(2-phenoxy-ethylsulfanylmethyl) -[1,3,4]oxadiazol-2-yl]-naphthalen-2-ylmethyl ester

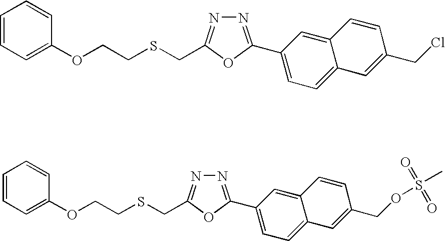

A dichloromethane suspension of {6-[5-(2-phenoxy-ethylsulfanylmethyl) -[1,3,4]oxadiazol-2-yl]-naphthalen-2-yl}-methanol (1.8 g, 4.59 mmol, 1 eq.) was treated with triethylamine (0.56 g, 5.51 mmol, 0.77 mL, 1.2 eq.) and methanesulfonyl chloride (0.63 g, 5.51 mmol, 0.43 mL, 1.2 eq.). The reaction was allowed to stir at room temperature and became a yellow solution. When complete, the reaction was washed with 0.1 M aqueous HCl. The organic layer was collected, dried over MgSO₄, filtered, and the solvent removed leaving a yellow oil which was purified via normal phase chromatography leaving 2-(6-chloromethyl-naphthalen-2-yl)-5-(2-phenoxy -ethylsulfanylmethyl)-[1,3,4]oxadiazole (0.71 g) as a white solid and methanesulfonic acid 6-[5-(2-phenoxy-ethylsulfanylmethyl)-[1,3,4]oxadiazol-2-yl]-naphthalen-2-ylmethyl ester (1.05 g) as a white solid.

2-(6-Chloromethyl-naphthalen-2-yl)-5-(2-phenoxy-ethylsulfanylmethyl) -[1,3,4]oxadiazole: ¹H NMR (d6-DMSO) δ 8.59 (s, 1H), 8.12 (m, 4H), 7.68 (m, 1H) , 7.26 (m, 2H), 6.93 (m, 3H), 4.98 (s, 2H), 4.28 (s, 2H), 4.22 (t, 2H, J=6.6 Hz), 3.06 (t, 2H, J=6.6 Hz) MS(ES⁺) m/z 411 [M+H]⁺.

Methanesulfonic acid 6-[5-(2-phenoxy-ethylsulfanylmethyl)-[1,3,4]oxadiazol-2-yl]-naphthalen-2-ylmethyl ester: ¹H NMR (d6-DMSO) δ 8.61 (s, 1H), 8.16 (m, 4H), 7.68 (m, 1H), 7.26 (m, 2H), 6.92 (m, 3H), 5.48 (s, 2H), 4.28 (s, 2H), 4.22 (t, 2H, J=6.6 Hz), 3.31 (s, 3H), 3.06 (t, 2H, J=6.6 Hz). MS(ES⁺) m/z 471 [M+H]⁺.

### Example 11

### Preparation of Dimethyl-{6-[5-(2-phenoxy-ethylsulfanylmethyl)-[1,3,4]oxadiazol-2-yl]-naphthalen-2-ylmethyl}-amine

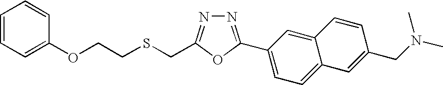

A THF solution of methanesulfonic acid 6-[5-(2-phenoxy-ethylsulfanylmethyl) -[1,3,4]oxadiazol-2-yl]-naphthalen-2-ylmethyl ester (0.5 g, 1.06 mmol, 1 eq.) was treated with dimethylanine (2M in THF, 1.11 mL, 2.23 mmol, 2.1 eq.) and stirred at 65° C. When complete, the reaction was diluted with dichloromethane and washed with 0.1 M aqueous NaOH. The organic layer was collected, dried, filtered, and the solvent removed in vacuo leaving a yellow oil that was purified by normal phase chromatography using 3.5% 2M NH3 in MeOH in chloroform as the mobile phase. Obtained a clear oil that was triturated with diethyl ether to obtain dimethyl-{⁶-[5-(2-phenoxy-ethylsulfanylmethyl) -[1,3,4]oxadiazol-2-yl]-naphthalen-2-ylmethyl}-amine (0.1429 g, 32% yield) as a white solid.

¹H NMR (d6-DMSO) δ 8.55 (s, 1H), 8.08 (m, 3H), 7.90 (s, 1H), 7.59 (m, 1H), 7.26 (m, 2H), 6.93 (m, 3H), 4.28 (s, 2H), 4.22 (t, 2H, J=6.6 Hz), 3.59 (s, 2H), 3.06 (t, 2H, J=6.6 Hz), 2.21 (s, 6H). MS(ES⁺) m/z 420 [M+H]⁺. IR (KBr, cm⁻) 3413.4, 2968.3, 2928.2, 2825.6, 2777.6, 1599.4, 1545.6, 1498.7, 1467.9, 1241.4, 1027.6, 903.3, 834.0, 762.9. Analytical composition calculated for C₂₄H₂₅N₃O₂S C, 68.71; H, 6.01; N, 10.02. Found C, 68.50; H, 6.04; N, 9.92. M.P. 95-96° C.

### Example 12

### Preparation of 2-(2-Phenoxy-ethylsulfanylmethyl)-5-(6-pyrrolidin-1-ylmethyl-naphthalen -2-yl)-[1,3,4]oxadiazole maleate

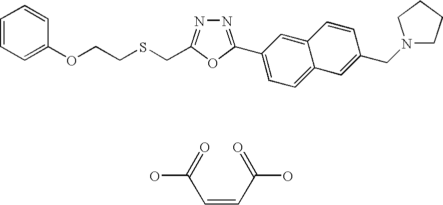

A THF solution of methanesulfonic acid 6-[5-(2-phenoxy-ethylsulfanylmethyl) -[1,3,4]oxadiazol-2-yl]-naphthalen-2-ylmethyl ester (0.5 g, 1.06 mmol, 1 eq.) was treated with pyrrolidine (0.16 g, 0.19 niL, 2.23 mmol, 2.1 eq.) as described for dimethyl-{6-[5-(2-phenoxy-ethylsulfanylmethyl)-[1,3,4]oxadiazol-2-yl]-naphthalen-2-ylmethyl }-amine to obtain a light yellow oil after purification. The oil was converted to the maleate salt by adding maleic acid in ethyl acetate to an ethyl acetate solution of the amine to give 2-(2-phenoxy-ethylsulfanylmethyl)-5-(6-pyrrolidin-1-ylmethyl-naphthalen-2-yl) -[1,3,4]oxadiazole maleate (0.5572 g, 93% yield) as a white solid collected by filtration.

¹H NMR (d6-DMSO) δ 8.64 (s, 1H), 8.24 (m, 1H), 8.14 (m, 3H), 7.74 (m, 1H), 7.26 (m, 2H), 6.93 (m, 3H), 6.03 (s, 2H), 4.54 (s, 2H), 4.29 (s, 2H), 4.22 (t, 2H, J=6.6 Hz), 3.23 (m, 4H), 3.06 (t, 2H, J=6.6 Hz), 1.95 (m, 4H). MS(ES⁺) rn/z 446 [M+H]⁺. IR (KBr, cm⁻¹) 3408.6, 2970.1, 2930.2, 2907.5, 2478.5, 1700.2, 1584.5, 1469.4, 1446.0, 1379.1, 1353.9, 1239.0, 1193.0, 1073.7, 1020.4, 864.3, 755.6, 722.3. Analytical composition calculated for C₃₀H₃₁N₃O₆S C, 64.16; H, 5.56; N, 7.48. Found C, 63.77; H, 5.51; N, 7.38. M.P. 133-135° C.

### Example 13

### Preparation of 1 -{6-[5-(2-phenoxy-ethylsulfanylmethyl)-[1,3,4]oxadiazol-2-yl]-naphthalen-2-ylmethyl}-piperidine

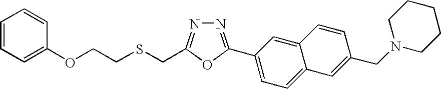

A THF solution of 2-(6-chloromethyl-naphthalen-2-yl)-5-(2-phenoxy -ethylsulfanylmethyl)-[1,3,4]oxadiazole (0.68 g, 1.65 mmol, 1 eq.) was treated with piperidine (0.30 g, 0.34 mL, 3.47 mmol, 2.1 eq.) as described for dimethyl-{6-[5-(2-phenoxy-ethylsulfanylmethyl)-[1,3,4]oxadiazol-2-yl]-naphthalen-2-ylmethyl}-amine to obtain a clear oil after purification which was triturated with diethyl ether to produce 1-{6-[5-(2-phenoxy-ethylsulfanylmethyl)-[1,3,4]oxadiazol-2-yl]-naphthalen-2-ylmethyl}-piperidine (0.5081 g, 67% yield) as a white solid.

¹H NMR (d6-DMSO) δ 8.54 (s, 1H), 8.07 (m, 3H), 7.89 (s, 1H), 7.59 (m, 1H), 7.26 (m, 2H), 6.93 (m, 3H), 4.27 (s, 2H), 4.22 (t, 2H, J=6.6 Hz), 3.61 (s, 2H), 3.06 (t, 2H, J=6.6 Hz), 2.37 (m, 4H), 1.52 (m, 4H), 1.40 (m, 2H). MS(ES⁺) m/z 460 [M+H]⁺. IR (KBr, cm−1) 2929.0, 2849.8, 2795.4, 2748.9, 1604.0, 1545.1, 1499.4, 1467.9, 1297.7, 1245.8, 1174.5, 1111.4, 1034.5, 894.9, 833.4, 748.3. Analytical composition calculated for C₂₇H₂₉N₃O₂S C, 70.56; H, 6.36; N, 9.14. Found C, 70.3 1; H, 6.36; N, 9.1 1. M.P. 93-94° C.

### Example 14

### Preparation of 2-(2-piperidinoethyl)-5-{2-[((2-phenoxyethyl)thio)methyl]-1,3,4-oxadiazol-5-yl}isoindolin-1-one

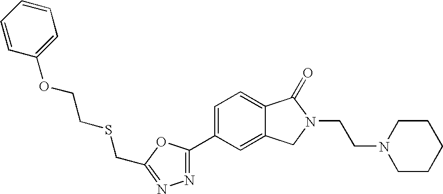

### a) 5-{2-[((2-Phenoxyethyl)thio)methyl]-1,3,4-oxadiazol-5-yl}phthalide

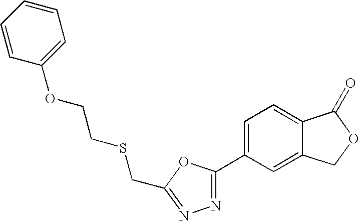

To a mixture of 4-carboxyphthalide (178 mg, 1 mmol), 2-phenoxythioacetic hydrazide hydrochloride (316 mg, 1.2 mmol), 4-(N,N -dimethylamino)phenyldiphenylphosphine (917 mg, 3 mmol), and triethylamine (607 mg, 6 mmol) in acetonitrile (10 mmol) was added carbon tetrachloride (770 mg, 5 mmol). The resultant mixture was stirred at room temperature overnight and concentrated. The residue was partitioned between ether (50 mL) and 2 M HCl (30 mL). The organic layer was washed with 2 M HCl (5×20 mL), dried (MgSO₄), and concentrated. The residue was triturated from methylene chloride and hexanes to give a white solid (186 mg, 51%). The reaction was repeated on 3 mmol scale to give the same product (652 mg, 59%).

¹H NMR (CDCI₃) δ 8.16 (d, 1H, J=8.1 Hz), 8.13 (s, 1H), 8.02 (d, 1H, J=7.8 Hz), 7.22-7.26 (m, 2H), 6.93 (t, 1H, J=7.3 Hz), 6.86 (d, 2H, J=8.0 Hz), 5.37 (s, 2H), 4.20 (t, 2H, J=5.8 Hz), 4.09 (s, 2H), 3.05 (t, 2H, J=5.9 Hz). MS (ES+) m/e 369 (M+1).

### b) 2-{[(2-Phenoxyethyl)thio]methyl}-5-{3-hydroxymethyl-4-[((2-piperidinoethyl)amino)carbonyl]phenyl}-1,3,4-oxadiazolo

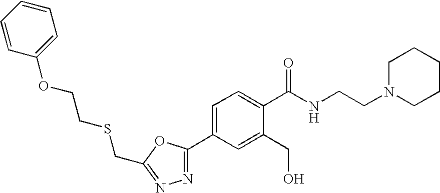

Lithium aluminum hydride (1 M in ether, 1 mL, 1 mmol) was diluted with tetrahydrofuran (1 mL) and N-(2-aminoethyl)piperidine (641 mg, 5 mmol) in tetrahydrofuran (1 mL) was added dropwise over 3 min. The resultant mixture was stirred at room temperature for 2 hr, diluted with tetrahydrofuran (4 mL), and 5-{2-[((2-phenoxyethyl)thio)methyl]-1,3,4-oxadiazol-5-yl}phthalide (368 mg, 1 mmol) was added. Stirring was continued at room temperature overnight and tetrahydrofuran (5 mL), followed by 2 M NaOH (5 mL) was added. The mixture was stirred for 30 min, diluted with water (15 mL), and extracted with ethyl acetate (3×15 mL). The combined ethyl acetate extracts were washed with brine (15 mL), dried (MgSO₄), and concentrated. The residue was recrystallized from methylene chloride and hexanes (1:1) to give a white solid (280 mg, 56%).

¹H NMR (CDC1₃) δ 8.02 (s, 1H), 8.0 (d, 1H, J=8.0 Hz), 7.64 (d, 1H, J=7.7 Hz), 7.22-7.26 (m, 2H), 7.10 (br s, 1H), 6.92 (t, 1H, J=7.3 Hz), 6.87 (d, 2H, J=8.4 Hz), 4.64 (s, 2H), 4.19 (t, 2H, J=6.0 Hz), 4.05 (s, 2H), 3.59 (dd, 2H, J-8.2, 5.5 Hz), 3.04 (t, 2H, J=5.9 Hz), 2.60 (t, 2H, J=5.7 Hz), 2.4-2.52 (m, 4H), 1.60-1.66 (m, 4H), 1.47-1.50 (m, 2H). IR (KBr, cm⁻¹) 3465, 3310, 2940, 2888, 2854, 1638, 1556, 1496, 1420, 1297, 1020, 750. MS (ES+) m/e 497 (M+1). Anal. Calcd for C₂₆H₃₂N₄O₄S: C, 62.88; H, 6.49; N, 11.28; S, 6.46. Found C, 63.27; H, 6.46; N, 11.14; S, 6.28.

### Preparation of 2-(2-piperidinoethyl)-5-{2-[((2-phenoxyethyl)thio)methyl]-1,3,4-oxadiazol-5-yl}isoindolin-1-one

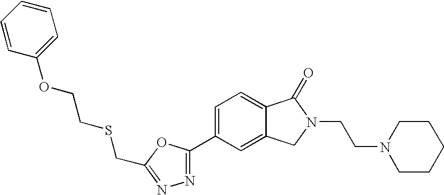

A mixture of 2-{[(2-Phenoxyethyl)thio]methyl) -5-{3-hydroxymethyl4-[((2-piperidinoethyl)amino)carbonyl]phenyl}-1,3,4-oxadiazolo (50 mg, 0.1 mmol) and triphenylphosphine (53 mg, 0.2 mmol) in methylene chloride (1 mL) was cooled to 0° C. and diethyl azodicarboxylate (35 mg, 0.2 mmol) in methylene chloride (0.5 mL) was added. After 15 min the cooling bath was removed. The mixture was stirred at room temperature overnight, concentrated and purified by chromatography (silica gel, 10% methanol/methylen chloride) to give the desired product (10 mg, 21%) and recovered starting benzyl alcohol (21 mg, 42%).

¹H NMR (CDC1₃) 6 8.11 (s, 1H), 8.08 (d, 1H, J=8.0 Hz), 7.93 (d, 1H, J=8.0 Hz), 7.22-7.26 (m, 2H), 6.92 (t, 1H, J=7.3 Hz), 6.86 (d, 2H, J=8.0 Hz), 4.58 (s, 2H), 4.19 (t, 2H, J=6.2 Hz), 4.06 (s, 2H), 3.75 (t, 2H, J=6.0 Hz), 3.04 (t, 2H, J=6.0 Hz), 2.60 (t, 2H, J=5.7 Hz), 2.39-2.45 (m, 4H), 1.52-1.60 (m, 4H), 1.39-1.43 (m, 2H). MS (ES+) m/e 479 (M+1). Anal. Calcd for C₂₆H₃₀N₄O₃S: C, 65.25; H, 6.32; N, 11.7 1; S, 6.70. Found C, 65.17; H, 6.31; N, 11.59; S, 6.52.

1. A compound of formula I: 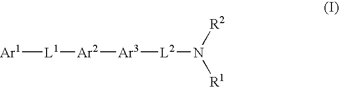
wherein:
Ar¹ is a cyclic group optionally substituted with one to five groups selected from C₁-C₈ alkyl, C₂-C₈ alkenyl, C₂-C₈ alkynyl, hydroxy, C₁-C₈ alkoxy, C₁-C₈ alkylaryl, phenyl, aryl, —O-aryl, heteroaryl, cycloalkyl, C₁-C₈ alkylcycloalkyl, cyano, —(CH₂)ₙNR⁶R⁶, C₁-C₈ haloalkyl, C₁-C₈ haloalkoxy, halo, (CH₂)ₙCOR⁶, (CH₂)ₙ NR⁵SO₂R ⁶, —(CH ₂)ₙC(O)NR⁶R⁶, heterocyclic, and C₁-C₈ alkylheterocyclic; wherein the cycloalkyl, phenyl, aryl, and heterocyclic groups are each optionally substituted with one to three groups independently selected from hydroxy, C₁-C₈ alkoxyalkyl, C₁-C₈ haloalkoxy, C₁-C₈ alkyl, halo, C₁-C₈ haloalkyl, nitro, cyano, amino, carboxamido, phenyl, aryl, alkylheterocyclic, heterocyclic, and oxo;
L¹ is a bond, —CH₂—, —CH₂CH₂—, —SCH₂—, —OCH₂—, —CH₂SCH₂—,or a divalent linker represented by the formula X₂—(CR³R⁴)ₘ—X₃ where X₂ is attached to Ar¹and X₃ is attached to Ar² wherein R³ and R⁴ are independently selected from a bond, hydrogen, C₁-C₈ alkyl, C₂-C₈ alkylene, C₂-C₈ alkynyl, phenyl, aryl, C₁-C₈ alkylaryl; wherein the alkyl, alkenyl, phenyl, and aryl groups are optionally substituted with one to five substitutents independently selected from oxo, nitro, cyano, C₁-C₈ alkyl, aryl, halo, hydroxy, C₁-C₈ alkoxy, C₁-C₈ halaoalkyl, (CH₂)ₙC(O)R⁶, and (CH₂)ₙCONR⁶R⁶;
X₂ is independently oxygen, —CH, —CONH(CR³R⁴)ₘ, —NHCO(CR³R⁴)ₘ, —(CR³R⁴)ₘ, —CHR⁶, —NR⁵, S, SO, SO₂, —O(CR³R⁴)ₘ, or —S(CR³R⁴)ₘ;
X₃ is independently oxygen, —C, —CH, —CHR⁶, —(CR³R⁴)ₘ, —NR⁵, S. SO, or SO₂;
Ar² is a 5-member monocyclic heterocyclic aromatic group or positional isomer thereof, having 1, 2, or 3 heteroatoms independently selected from nitrogen, oxygen and sulfur; and
wherein Ar² is optionally substituted with one to three substitutents independently selected from C₁-C₈ alkyl, C₂-C₈ alkenyl, C₂-C₈ alkynyl, hydroxy, C_{-C}₈ alkoxy, C₁-C₈ alkylaryl, phenyl, aryl, C₃-C₈ cycloalkyl, C₁-C₈ alkylcycloalkyl, cyano, C₁-C₈ haloalkyl, halo, (CH₂)ₙ,C(O)R⁶, (CH₂)ₙ,C(O)OR⁶, (CH₂)ₙ,NR⁵SO₂R⁶, (CH₂)ₙC(O)NR⁶R⁶, and C₁-C₈ alkylheterocyclic;
Ar³ is an optionally substituted bicyclic aromatic or non-aromatic group;
L² is —CH₂—, —CH₂CH₂—or a divalent linker represented by the formula X₄—(CR³R⁴)ₘ—X₅;
wherein X₄ is selected from the group consisting of C, —CH, CHR⁶, —CO, O, —NR⁵, —NC(O)—, —NC(S), —C(O)NR⁵—, —NR^{6′}C(O)NR⁶, —NR^{6′}C(S)NR⁶, —SO₂NR⁷, —NRSO₂R⁷, and —NR^{6′}C(NR⁵)NR⁶;
X₅ is selected from the group consisting of O, —CH₂, —CH, —O(CR³R⁴)m, NR³(CR³(CR³R⁴)ₘ, SO, SO₂, S, and SCH₂; wherein the group X₄—(CR ³R⁴)ₘ—X₅imparts stability to the compound of formula (1) and may be a saturated or unsaturated chain or divalent linker;
R¹ and R² are independently hydrogen, C₁-C₈ alkyl, C₂-C₈ alkenyl, C₃-C₈ cycloalkyl, C₁-C₈ alkylaryl, —C(O)C₁-C₈ alkyl, —C(O)OC₁-C₈ alkyl, C₁—C₈ alkylcycloalkyl, (CH₂)ₙC(O)OR⁵, (CH₂)ₙC(O)R⁵, (CH₂)ₙC(O)NR ⁶R⁶, and (CH₂)ₙNSO₂R⁵; wherein each of the alkyl, alkenyl, aryl are each optionally substituted with one to five groups independently selected from C₁-C₈ alkyl, C₂-C₈ alkenyl, phenyl, and alkylaryl; and wherein R¹ and R² may combine together, and with the nitrogen atom to which they are attached or with 0, 1, 2 or 3 atoms adjacent to the nitrogen atom to form a nitrogen containing heterocycle which may have 1, or 2 substituents independently selected from C₁-C₈ alkyl, C₂-C₈ alkenyl, C₃-C₈ cycloalkyl, C_{-C}₁-C₈alkylaryl, —C(O)C₁-C₈ alkyl, —C(O)OC₁-C₈ alkyl, C₁-C₈ alkylcycloalkyl,oxo, halo amino, and (CH₂)ₙC(O)NR⁶R⁶;
R⁵ is hydrogen, CN, C₁-C₈ alkyl, C₂-C₈ alkenyl, C₅-C₈ alkylaryl, (CH₂)ₙNSO ₂C₁-C₈ alkyl; (CH₂)ₙNSO₂phenyl, (CH₂)ₙ,NSO₂aryl, —C(O)C₁-C₈ alkyl, or —C(O)OC,-C₈ alkyl; and
R⁶ and R^{6′}are each independently hydrogen, C₁-C₈ alkyl, phenyl, aryl, C₁-C₈alkylaryl, C₁-C₈alkylcycloalkyl, or C₃-C₈cycloalkyl;
R⁷ is hydrogen, C₁-C₈ alkyl, phenyl, aryl, C₁-C₈alkylaryl, or C₃-C₈cycloalkyl, and wherein m is an integer from 1 to 8; and n is an integer from 0 to 8; or a pharmaceutically acceptable salt, solvate, racemate, or enantiomer diastereomer or mixture of diastereomers thereof.

2. A compound according to claim 1 wherein the group Ar¹ is selected from the group consisting of: phenyl, benzothiophene, benzofuran, or naphthyl.

3. A compound according to claim 1 wherein the group L¹ is a linker selected from the group consisting of: —CH₂—, —CH₂CH₂—, —CH₂CH₂CH₂—, —SCH₂—, —OCH₂—, —CH₂SCH₂—, —CH₂OCH₂—, or —OCH₂CH₂SCH₂—.

4. A compound according to claim 1 wherein Ar³ is an aromatic group selected from the group consisiting of: indole, naphthyl, tetrahydronaphthyl, isoindolinone, isoquinolone, benzothiophene, or benzofuran.

5. A compound of claim 1 wherein Ar² is a 4 or 5 member aromatic group selected from the group consisting of: oxazole, oxadiazole, or furan.

6. A compound according to claim 1 wherein the linker (L²) is: —CH₂—, —CH₂CH₂—, or —CH₂CH₂CH₂—.

7. A compound according to claim 1 wherein R¹ and R² combine with the nitrogen atom to form piperidinyl, pyrrolidinyl, azepine, or azetidinyl.

8. A.compound according to claim 1 wherein R¹ and R² are independently selected from the group consisting of hydrogen, methyl, ethyl, propyl, isopropyl, methylcyclopentane, methylcyclohexane, phenyl, benzyl, cyclopentyl, cyclohexyl, methylcyclopropane and methylcyclobutane.

9. (canceled)

10. (canceled)

11. (canceled)

12. A compound according to claim 1 wherein at least one of L¹ and L² has a chain length of 3 to 5 atoms.

13. A compound selected from the group consisting of: Dimethyl-{6-[5-(2-phenoxy-ethylsulfanylmethyl)-[1,3,4]oxadiazol-2-yl]-benzofuran-2-ylmethyl}-amine oxalate,
Dimethyl-{5-[5-(2-phenoxy-ethylsulfanylmethyl)-[1,3,4]oxadiazol-2-yl]-benzofuran-2-ylmethyl}-amine oxalate,
{-Methanesulfonyl-5-[5-(2-phenoxy-ethylsulfanylmethyl)-[1,3,4]oxadiazol-2-yl]-1H-indol 2-ylmethyl}-dimethyl-amine,
Dimethyl-{5-[5-(2-phenoxy-ethylsulfanylmethyl)-[1,3,4]oxadiazol-2-yl]-1H-indol-2-ylmethyl}-amine oxalate,
{1 -Methanesulfonyl-6-[5-(2-phenoxy-ethylsulfanylmethyl)-[1,3,4]oxadiazol-2yl]-1H-indol- 2-ylmethyl}-dimethyl-amine,
Dimethyl- {16-[5-(2-phenoxy-ethylsulfanylmethyl)-[1,3,4]oxadiazol-2-yl]-1 H-indol-2-ylmethyl}-amine,
Dimethyl-{-methyl-6-[5-(2-phenoxy-ethylsulfanylmethyl)-[1,3,4]oxadiazol-2-yl]-1H-indol -2-ylmethyl}-amine oxalate,
Dimethyl-{5-[5-(2-phenoxy-ethylsulfanylmethyl)-[1,3,4]oxadiazol-2-yl]-1H-indol-3-ylmethyl}-amine oxalate,
Dimethyl-{6-[5-(²-phenoxy-ethylsulfanylmethyl)-[1,3,4]oxadiazol-2-yl]-1H-indol-3-ylmethyl}-amine maleate,
Dimethyl-{1-methyl-5-[5-(2-phenoxy-ethylsulfanylmethyl)-[1,3,4]oxadiazol-2-yl]-1H-indo -3-ylmethyl}-amine oxalate,
Dimethyl-{4-[5-(2-phenoxy-ethylsulfanylmethyl)-[1,3,4]oxadiazol-2-yl]-naphthalen-1-yl}-amine,
Dimethyl-{6-[5-(2-phenoxy-ethylsulfanylmethyl)-[1,3 ,4]oxadiazol-2-yl]-naphthalen-2-ylmethyl}-amine,
2-(2-Phenoxy-ethylsulfanylmethyl)-5-(6-pyrrolidin-1 -ylmethyl-naphthalen-2-yl) -[1,3,4]oxadiazole maleate,
1 -{6-[5-(2-phenoxy-ethylsulfanylmethyl)-[1,3,4]oxadiazol-2-yl]-naphthalen-2-ylmethyl}-piperidine,
2-(2-piperidinoethyl)-5-55 2-[((2-phenoxyethyl)thio)methyl]-1,3,4-oxadiazol-5-yl)}isoindolin -1-one,
and pharmaceutically acceptable salt, solvate, enatiomer, prodrug, diastereomer or mixture thereof.

14. A compound selected from the group consisting of: 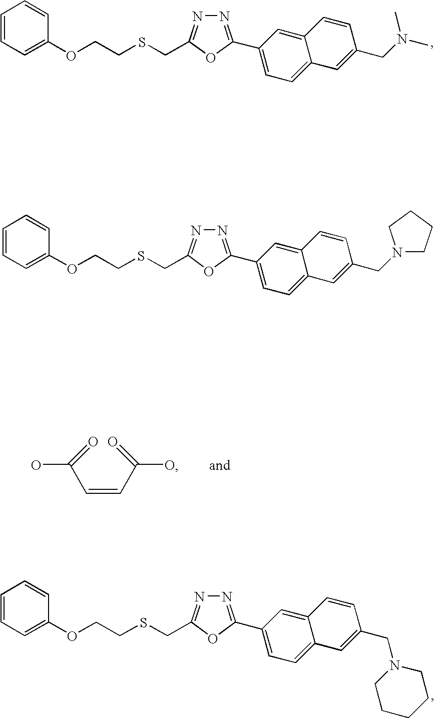
or pharmaceutically acceptable salt, racemate, solvate, enantioner or diastereomeror mixture of diastereomers thereof.

15. (canceled)

16. A method of treating Type II Diabetes comprising administering to a patient in need thereof a compound of claim 1.

17. A method of treating obesity and Related Diseases comprising administering to a patient in need thereof a compound of claim 1.

18. (canceled)

19. A pharmaceutical formulation comprising a compound of claim 1 and a pharmaceutical carrier.

20. (canceled)
